(19) United States
(12) Patent Application Publication
(10) Pub. No.: US 2025/0354140 A1
(43) Pub. Date: Nov. 20, 2025

(54) CALCIUM SALTED SPHERICAL NUCLEIC ACIDS

(71) Applicant: NORTHWESTERN UNIVERSITY, Evanston, IL (US)

(72) Inventors: Chad A. Mirkin, Wilmette, IL (US); Jungsoo Park, Evanston, IL (US)

(21) Appl. No.: 18/862,866

(22) PCT Filed: May 5, 2023
(86) PCT No.: PCT/US2023/066675

§ 371 Date: Nov. 4, 2024

Related U.S. Application Data
(60) Provisional application No. 63/338,736, filed on May 5, 2022.

(51) Int. Cl. C12N 15/11; A61K 9/51; A61K 31/7088; A61K 48/00; C12Q 1/68

(52) U.S. Cl. CPC C12N 15/111; A61K 9/5192; A61K 31/7088; A61K 48/00; C12Q 1/68; C12N 2310/14; C12N 2310/351; C12N 2310/51; C12N 2320/30

(57) ABSTRACT

The disclosure is generally related to calcium salted spherical nucleic acids (SNAs). SNAs comprise a nanoparticle core surrounded by a shell of oligonucleotides. In some aspects, the disclosure provides a spherical nucleic acid (SNA) comprising: (a) a nanoparticle core; and (b) a shell of oligonucleotides attached to the external surface of the nanoparticle core, wherein one or more oligonucleotides in the shell of oligonucleotides comprises a phosphate backbone; the SNA comprising Ca²⁺ ions adsorbed to the phosphate backbone of one or more oligonucleotides in the shell of oligonucleotides. Methods of making and using the SNAs are also provided herein.

### CROSS-REFERENCE TO RELATED APPLICATIONS

This application is a U.S. National Phase of International Application No. PCT/US23/66675, filed May 5, 2023, which claims the priority benefit under 35 U.S.C. § 119(e) of U.S. Provisional Patent Application No. 63/338,736, filed May 5, 2022, which is incorporated herein by reference in its entirety.

### STATEMENT OF GOVERNMENT INTEREST

This invention was made with government support under grant numbers 5U54CA199091-05, 5R01CA208783-05, and P50CA221747 awarded by the National Institutes of Health (NIH). The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2022-019_Seqlisting.XML”, which was created on Apr. 27, 2023 and is 49,675 bytes in size. The subject matter of the Sequence Listing is incorporated herein in its entirety by reference.

### FIELD

The disclosure is generally related to calcium salted spherical nucleic acids (SNAs). SNAs comprise a nanoparticle core surrounded by a shell of oligonucleotides. Methods of making and using the SNAs are also provided herein.

### BACKGROUND

Development of therapeutic inhibitory oligonucleotides (e.g., siRNAs) has been limited because inhibitory oligonucleotides on their own cannot enter the target cells due to their poor stability in the blood stream and rapid clearance from circulation, rendering them impotent for systemic delivery.

### SUMMARY

Spherical nucleic acids (SNAs) provide distinct properties to overcome the challenges of using inhibitory oligonucleotides compared to their linear counterpart with enhanced cellular uptake and resistance to nuclease degradation. However, inhibitory oligonucleotide sequence dependent and cell-line differences can lead to decreased gene regulation efficiency of the inhibitory oligonucleotide functionalized SNA construct. In some aspects, the present disclosure provides CaCl₂) salted SNAs that significantly improve the gene regulation activity of the SNA by more than 20-fold independent of the inhibitory oligonucleotide sequence functionalized to the SNA surface. Improved gene regulation efficiency of the CaCl₂) salted inhibitory oligonucleotide-SNAs provide for the development and commercialization of SNAs that target a variety of genes involved in multiple disorders, including cancers and various genetic diseases.

Applications of the technology described herein include, but are not limited to:
RNA interference
Therapeutic gene modulation
Cytosolic mRNA detection

Advantage of the technology described herein include, but are not limited to:
Significantly improves the gene regulation activity of SNAs (e.g., PLGA SNAs)
Enhances cellular uptake of SNA
Use of a readily available calcium chloride (CaCl₂)) instead of relatively expensive cationic polymers or peptides
Easy, Straightforward synthesis
No cellular toxicity

Accordingly, in some aspects the disclosure provides a spherical nucleic acid (SNA) comprising: (a) a nanoparticle core; and (b) a shell of oligonucleotides attached to the external surface of the nanoparticle core, wherein each oligonucleotide in the shell of oligonucleotides comprises a phosphate backbone; the SNA comprising Ca²⁺ ions adsorbed to the phosphate backbone of one or more oligonucleotides in the shell of oligonucleotides. In any of the aspects or embodiments of the disclosure, Ca²⁺ ions are adsorbed to the phosphate backbone of each oligonucleotide in the shell of oligonucleotides. In further aspects, the disclosure provides a spherical nucleic acid (SNA) comprising: (a) a nanoparticle core; and (b) a shell of oligonucleotides attached to the external surface of the nanoparticle core, wherein one or more oligonucleotides in the shell of oligonucleotides comprises a phosphate backbone; the SNA comprising Ca²⁺ ions adsorbed to one or more oligonucleotides in the shell of oligonucleotides. In some embodiments, each oligonucleotide in the shell of oligonucleotides comprises a phosphate backbone. In further embodiments, Ca²⁺ ions are adsorbed to the phosphate backbone of one or more oligonucleotides in the shell of oligonucleotides. In various embodiments, Ca²⁺ ions are adsorbed to the phosphate backbone of each oligonucleotide in the shell of oligonucleotides. In some aspects or embodiments of the disclosure, Ca²⁺ ions are adsorbed to one or more nucleobases of one or more oligonucleotides in the shell of oligonucleotides. In various embodiments, the SNA has a zeta potential that is about −40 millivolts (mV) to about −10 mV. In some embodiments, the SNA has a zeta potential that is about −10 mV. In various embodiments, the nanoparticle core is a metallic core, a semiconductor core, an insulator core, an upconverting core, a micellar core, a dendrimer core, a liposomal core, a lipid nanoparticle core, a polymer core, a metal-organic framework core, a protein core, or a combination thereof. In some embodiments, the polymer is polylactide, a polylactide-polyglycolide copolymer, a polycaprolactone, a polyacrylate, alginate, albumin, polypyrrole, polythiophene, polyaniline, polyethylenimine, poly(methyl methacrylate), poly(lactic-co-glycolic acid) (PLGA), or chitosan. In further embodiments, the nanoparticle core is gold, silver, platinum, aluminum, palladium, copper, cobalt, indium, cadmium selenide, iron oxide, fullerene, metal-organic framework, silica, zinc sulfide, or nickel. In some embodiments, the nanoparticle core is a liposome. In further embodiments, the liposome comprises a lipid selected from the group consisting of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), and cholesterol. In some embodiments, one or more oligonucleotides in the shell of oligonucleotides is attached to the external surface of the nanoparticle core through a lipid anchor group. In further embodiments, the lipid anchor group is attached to the 5′ end or the 3′ end of the one or more oligonucleotides. In further embodiments, the lipid anchor group is tocopherol or cholesterol. In some embodiments, one or more oligonucleotides in the shell of oligonucleotides is modified on its 5′ end and/or 3′ end with dibenzocyclooctyl (DBCO). In some embodiments, one or more oligonucleotides in the shell of oligonucleotides is modified on its 5′ end and/or 3′ end with a thiol. In further embodiments, the nanoparticle core and one or more or oligonucleotides in the shell of oligonucleotides comprise complementary reactive moieties that together form a covalent bond. In still further embodiments, the reactive moiety on the nanoparticle core comprises an azide, an alkyne, a maleimide, a thiol, an alcohol, an amine, a carboxylic acid, an olefin, an isothiocyanate, a N-hydroxysuccinimide, a phosphine, a nitrone, a norbornene, an oxanorbornene, a transcycloctene, an s-tetrazene, an isocyanide, a tetrazole, a nitrile oxide, a quadricyclane, or a carbodiimide. In some embodiments, the reactive moiety on the one or more or each oligonucleotide in the shell of oligonucleotides is on a terminus of the oligonucleotide. In further embodiments, the reactive moiety on the one or more or each oligonucleotide in the shell of oligonucleotides comprises an alkyne, an azide, a maleimide, a thiol, an alcohol, an amine, a carboxylic acid, an olefin, an isothiocyanate, a N-hydroxysuccinimide, a phosphine, a nitrone, a norbornene, an oxanorbornene, a transcycloctene, an s-tetrazene, an isocyanide, a tetrazole, a nitrile oxide, a quadricyclane, or a carbodiimide. In still further embodiments, the alkyne comprises dibenzocyclooctyl (DBCO) alkyne or a terminal alkyne. In some embodiments, the nanoparticle core comprises an azide reactive moiety and the one or more or oligonucleotides in the shell of oligonucleotides comprises an alkyne reactive moiety, or vice versa. In some embodiments, the alkyne reactive moiety comprises a DBCO alkyne. In some embodiments, the shell of oligonucleotides comprises about 5 to about 600 oligonucleotides. In further embodiments, the shell of oligonucleotides comprises about 5 to about 500 oligonucleotides. In still further embodiments, the shell of oligonucleotides comprises about 75 to about 100 oligonucleotides. In some embodiments, each oligonucleotide in the shell of oligonucleotides is about 5 to about 100 nucleotides in length. In some embodiments, each oligonucleotide in the shell of oligonucleotides is about 10 to about 50 nucleotides in length. In further embodiments, each oligonucleotide in the shell of oligonucleotides is about 20 to about 25 nucleotides in length. In some embodiments, each oligonucleotide in the shell of oligonucleotides has the same nucleotide sequence. In some embodiments, the shell of oligonucleotides comprises at least two oligonucleotides having different nucleotide sequences. In various embodiments, the shell of oligonucleotides is comprised of single-stranded DNA oligonucleotides, double-stranded DNA oligonucleotides, or a combination thereof. In further embodiments, the shell of oligonucleotides is comprised of single-stranded, double-stranded RNA oligonucleotides, or a combination thereof. In some embodiments, at least one oligonucleotide in the shell of oligonucleotides comprises a detectable marker. In some embodiments, the at least one oligonucleotide in the shell of oligonucleotides is configured to associate (e.g., hybridize) to a target analyte. In some embodiments, each oligonucleotide in the shell of oligonucleotides is configured to associate (e.g., hybridize) to a target analyte. In some embodiments, the detectable marker is attached to a polynucleotide hybridized to the at least one oligonucleotide in the shell of oligonucleotides. In some embodiments, the detectable marker attached to the polynucleotide is quenched when the polynucleotide with the detectable marker is hybridized to the at least one oligonucleotides in the shell of oligonucleotides. In some embodiments, the detectable marker attached to the polynucleotide is quenched when the polynucleotide with the detectable marker is hybridized to each oligonucleotide in the shell of oligonucleotides. In various embodiments, the shell of oligonucleotides comprises an inhibitory oligonucleotide, an immunostimulatory oligonucleotide, a targeting oligonucleotide, or a combination thereof. In further embodiments, the inhibitory oligonucleotide is an antisense oligonucleotide, small interfering RNA (siRNA), an aptamer, a short hairpin RNA (shRNA), a DNAzyme, or an aptazyme. In some embodiments, the immunostimulatory oligonucleotide comprises a CpG nucleotide sequence. In further embodiments, the immunostimulatory oligonucleotide is a toll-like receptor (TLR) agonist. In still further embodiments, the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13). In some embodiments, the TLR is TLR9. In some embodiments, one or more oligonucleotides in the shell of oligonucleotides comprises or consists of the sequence of 5′-TCCATGACGTTCCTGACGTT-3′ (SEQ ID NO: 1). In some embodiments, one or more oligonucleotides in the shell of oligonucleotides comprises or consists of the sequence of 5′-TCGTCGTTTTGTCGTTTTGTCGTT-3′ (SEQ ID NO: 3). In further embodiments, one or more oligonucleotides in the shell of oligonucleotides comprises or consists of the sequence of 5′-TCCATGACGTTCCTGACGTT (Spacer-18 (hexaethyleneglycol))₂Cholesterol-3′ (SEQ ID NO: 2). In still further embodiments, one or more oligonucleotides in the shell of oligonucleotides comprises or consists of the sequence of 5′-TCGTCGTTTTGTCGTTTTGTCGTT (Spacer-18 (hexaethyleneglycol))₂Cholesterol-3′ (SEQ ID NO: 4). In some embodiments, the SNA has an intercalating dye intensity that is decreased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to intercalating dye intensity of a NaCl-salted SNA under identical conditions.

In some aspects, the disclosure provides a composition comprising a plurality of the spherical nucleic acids (SNAs) of the disclosure. In some embodiments, the composition further comprises a therapeutic agent.

The disclosure also provides, in various aspects, a method of making a calcium chloride (CaCl₂)) salted spherical nucleic acid (SNA), the SNA comprising: (a) a nanoparticle core; and (b) a shell of oligonucleotides attached to the external surface of the nanoparticle core, wherein each oligonucleotide in the shell of oligonucleotides comprises a phosphate backbone; the method comprising: combining the nanoparticle core, a plurality of oligonucleotides, and calcium chloride (CaCl₂)) to create a mixture, wherein the combining results in the plurality of oligonucleotides becoming attached to the nanoparticle core to create the shell of oligonucleotides attached to the external surface of the nanoparticle core, thereby resulting in the CaCl₂) salted SNA. In some embodiments, the method further comprises isolating the CaCl₂) salted SNA from the mixture. Thus, in some aspects, the disclosure provides a method of making a calcium chloride (CaCl₂)) salted spherical nucleic acid (SNA), the SNA comprising: (a) a nanoparticle core; and (b) a shell of oligonucleotides attached to the external surface of the nanoparticle core, wherein each oligonucleotide in the shell of oligonucleotides comprises a phosphate backbone; the method comprising: combining the nanoparticle core, a plurality of oligonucleotides, and calcium chloride (CaCl₂)) to create a mixture, wherein the combining results in the plurality of oligonucleotides becoming attached to the nanoparticle core to create the shell of oligonucleotides attached to the external surface of the nanoparticle core, thereby resulting in the CaCl₂) salted SNA, and then optionally isolating the CaCl₂) salted SNA from the mixture. In some embodiments, Ca²⁺ ions are adsorbed to the phosphate backbone of each oligonucleotide in the shell of oligonucleotides of the CaCl₂) salted SNA. In various embodiments, the CacI₂ salted SNA has a zeta potential that is about −40 millivolts (mV) to about −10 mV. In further embodiments, the CaCl₂) salted SNA has a zeta potential that is about −10 millivolts (mV). In some embodiments, concentration of CaCl₂) in the mixture is 70 millimolar (mM) to about 350 mM. In further embodiments, concentration of CaCl₂) in the mixture is about 230 millimolar (mM). In various embodiments, the nanoparticle core is a metallic core, a semiconductor core, an insulator core, an upconverting core, a micellar core, a dendrimer core, a liposomal core, a polymer core, a metal-organic framework core, a protein core, or a combination thereof. In some embodiments, the polymer is polylactide, a polylactide-polyglycolide copolymer, a polycaprolactone, a polyacrylate, alginate, albumin, polypyrrole, polythiophene, polyaniline, polyethylenimine, poly(methyl methacrylate), poly(lactic-co-glycolic acid) (PLGA), or chitosan. In further embodiments, the nanoparticle core is gold, silver, platinum, aluminum, palladium, copper, cobalt, indium, cadmium selenide, iron oxide, fullerene, metal-organic framework, silica, zinc sulfide, or nickel. In various embodiments, the shell of oligonucleotides comprises about 5 to about 600 oligonucleotides. In some embodiments, the shell of oligonucleotides comprises about 5 to about 500 oligonucleotides. In some embodiments, the shell of oligonucleotides comprises about 75 to about 100 oligonucleotides. In further embodiments, each oligonucleotide in the shell of oligonucleotides is about 5 to about 100 nucleotides in length. In various embodiments, each oligonucleotide in the shell of oligonucleotides is about 10 to about 50 nucleotides in length. In some embodiments, each oligonucleotide in the shell of oligonucleotides is about 25 nucleotides in length. In some embodiments, each oligonucleotide in the shell of oligonucleotides has the same nucleotide sequence. In some embodiments, the shell of oligonucleotides comprises at least two oligonucleotides having different nucleotide sequences. In various embodiments, the shell of oligonucleotides is comprised of single-stranded DNA oligonucleotides, double-stranded DNA oligonucleotides, or a combination thereof. In some embodiments, the shell of oligonucleotides is comprised of single-stranded, double-stranded RNA oligonucleotides, or a combination thereof. In further embodiments, the shell of oligonucleotides is comprised of single-stranded DNA oligonucleotides, double-stranded DNA oligonucleotides, single-stranded RNA oligonucleotides, double-stranded RNA oligonucleotides, or a combination thereof. In further embodiments, at least one oligonucleotide in the shell of oligonucleotides comprises a detectable marker. In various embodiments, the shell of oligonucleotides comprises an inhibitory oligonucleotide, an immunostimulatory oligonucleotide, or a combination thereof. In further embodiments, the inhibitory oligonucleotide is an antisense oligonucleotide, small interfering RNA (siRNA), an aptamer, a short hairpin RNA (shRNA), a DNAzyme, or an aptazyme. In some embodiments, the immunostimulatory oligonucleotide comprises a CpG nucleotide sequence. In further embodiments, the immunostimulatory oligonucleotide is a toll-like receptor (TLR) agonist. In still further embodiments, the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13). In some embodiments, the TLR is TLR9. In further embodiments, one or more oligonucleotides in the shell of oligonucleotides comprises or consists of the sequence of 5′-TCCATGACGTTCCTGACGTT-3′ (SEQ ID NO: 1). In further embodiments, one or more oligonucleotides in the shell of oligonucleotides comprises or consists of the sequence of 5′-TCGTCGTTTTGTCGTTTTGTCGTT-3′ (SEQ ID NO: 3). In some embodiments, one or more oligonucleotides in the shell of oligonucleotides comprises or consists of the sequence of 5′-TCCATGACGTTCCTGACGTT (Spacer-18 (hexaethyleneglycol))₂Cholesterol-3′ (SEQ ID NO: 2). In some embodiments, one or more oligonucleotides in the shell of oligonucleotides comprises or consists of the sequence of 5′-TCGTCGTTTTGTCGTTTTGTCGTT (Spacer-18 (hexaethyleneglycol))₂Cholesterol-3′ (SEQ ID NO: 4). In various embodiments, the CaCl₂) salted SNA is an SNA as described herein.

In further aspects, the disclosure provides a composition comprising a plurality of the CaCl₂) salted spherical nucleic acids (SNAs) produced by a method of the disclosure. In some embodiments, the composition further comprises a therapeutic agent.

In some aspects, the disclosure provides a method of inhibiting expression of a gene product comprising the step of hybridizing a target polynucleotide encoding the gene product with a CaCl₂) salted spherical nucleic acid (SNA) or composition of the disclosure, wherein hybridizing between the target polynucleotide and one or more oligonucleotides in the shell of oligonucleotides occurs over a length of the target polynucleotide with a degree of complementarity sufficient to inhibit expression of the gene product. In some embodiments, expression of the gene product is inhibited in vivo. In some embodiments, expression of the gene product is inhibited in vitro. In various embodiments, expression of the gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to inhibition of gene product expression of a spherical nucleic acid (SNA) comprising a nanoparticle core and a shell of oligonucleotides attached to the external surface of the nanoparticle core, wherein each oligonucleotide in the shell of oligonucleotides comprises a phosphate backbone, and wherein the SNA does not comprise Ca²⁺ ions adsorbed to the phosphate backbone of one or more oligonucleotides in the shell of oligonucleotides. In some embodiments, expression of the gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to inhibition of gene product expression of a sodium chloride (NaCl)-salted spherical nucleic acid (SNA) under identical conditions. In further embodiments, the hybridizing occurs intracellularly. In some embodiments, accumulation of a spherical nucleic acid (SNA) of the disclosure within an endosome is reduced by at least 5%, at least 10%, at least 15%, at least 20%, at least 25%, at least 30% or more compared to accumulation of NaCl-salted SNAs within the endosome.

In further aspects, the disclosure provides a method of inhibiting expression of a gene product comprising the step of contacting a cell comprising the gene product with a SNA of the disclosure, thereby inhibiting expression of the gene product. In some embodiments, expression of the gene product is inhibited in vivo. In some embodiments, expression of the gene product is inhibited in vitro. In various embodiments, expression of the gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to inhibition of gene product expression in a cell contacted with a NaCl-salted spherical nucleic acid (SNA) under identical conditions. In some embodiments, expression of the gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to inhibition of gene product expression in a cell not contacted with a spherical nucleic acid (SNA). In further embodiments, accumulation of a spherical nucleic acid (SNA) of the disclosure within an endosome is reduced by at least 5%, at least 10%, at least 15%, at least 20%, at least 25%, at least 30% or more compared to accumulation of NaCl-salted SNAs within the endosome.

In further aspects, the disclosure provides a method of treating a disorder comprising administering an effective amount of a CaCl₂) salted SNA or composition of the disclosure to a subject in need thereof, wherein the administering treats the disorder. In various embodiments, the disorder is cancer, an infectious disease, an autoimmune disease, or a combination thereof.

In some aspects, the disclosure provides a method for detecting a target analyte comprising the step of contacting the target analyte with a CaCl₂) salted SNA or composition of the disclosure, wherein one or more oligonucleotides in the shell of oligonucleotides comprises a detectable marker, wherein the contacting results in binding of the target analyte to the one or more oligonucleotides in the shell of oligonucleotides comprising the detectable marker, resulting in a detectable change and thereby detecting the target analyte. In some embodiments, the detectable marker is attached to a polynucleotide hybridized to the one or more oligonucleotides in the shell of oligonucleotides, wherein association of the one or more oligonucleotides in the shell of oligonucleotides with the target analyte releases the polynucleotide hybridized to the one or more oligonucleotides in the shell of oligonucleotides and the detectable marker is detectable after release. In further embodiments, the detectable marker attached to the polynucleotide is quenched when the polynucleotide with the detectable marker is hybridized to the one or more oligonucleotides in the shell of oligonucleotides. In still further embodiments, the detectable marker is detectable only when the one or more oligonucleotides in the shell of oligonucleotides is associated with the target analyte. In some embodiments, the detectable marker is quenched when the one or more oligonucleotides in the shell of oligonucleotides is not associated with the target analyte. In various embodiments, the detectable marker is situated at an internal location within the oligonucleotide. In some embodiments, the binding results in restriction of internal rotation of the detectable marker. In further embodiments, the detectable marker is thiazole orange (TO), quinoline blue, quinoline violet, thiazole red, a derivative thereof, or a cyanine derivative. In some embodiments, the detectable change is proportional to concentration of the target analyte. In various embodiments, the target analyte is a protein, an ion, a small molecule, a lipid, a carbohydrate, an oligosaccharide, a cell, an oligonucleotide, or a combination thereof. In further embodiments, the target analyte is RNA. In some embodiments, the target analyte is mRNA. In some embodiments, the target analyte is cytosolic mRNA.

In further aspects, the disclosure provides a method for up-regulating activity of a toll-like receptor (TLR), comprising contacting a cell having the toll-like receptor with a CaCl₂) salted SNA or composition of the disclosure, thereby up-regulating activity of the TLR. In some embodiments, the shell of oligonucleotides comprises one or more oligonucleotides that is a TLR agonist. In further embodiments, the toll-like receptor is toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, toll-like receptor 13, or a combination thereof. In some embodiments, the method is performed in vitro. In some embodiments, the method is performed in vivo.

In yet additional aspects, the disclosure provides a method for down-regulating activity of a toll-like receptor (TLR), comprising contacting a cell having the toll-like receptor with a CaCl₂) salted SNA or composition of the disclosure, thereby down-regulating activity of the TLR. In some embodiments, the shell of oligonucleotides comprises one or more oligonucleotides that is a TLR antagonist. In various embodiments, the toll-like receptor is toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, toll-like receptor 13, or a combination thereof. In some embodiments, the method is performed in vitro. In some embodiments, the method is performed in vivo.

### BRIEF DESCRIPTION OF THE DRAWINGS

FIG. 1 depicts a synthesis scheme of siRNA duplex functionalized PLGA SNAs.

FIG. 2 shows that CaCl₂) salted SNAs did not have change in size but did show a change in zeta potential.

FIGS. 3A and 3B show that CaCl₂) Salted PLGA SNAs were able to down-regulate Luc2 expression in a CaCl₂) dose dependent manner. In FIG. 3A, cells were treated with concentrations of CaCl₂) salted SNAs as indicated along the bottom of the x-axis.

FIG. 4 shows CaCl₂) salted PLGA SNAs down-regulated Luc2 Expression in U87 reporter cell line.

FIG. 5 shows that CaCl₂) Salted PLGA SNAs did not induce significant cellular toxicity.

FIG. 6 demonstrates that CaCl₂) salted PLGA SNAs showed enhanced down regulation of IDH1 in U87 cell line.

FIG. 7 shows that CaCl₂) salted PLGA SNAs showed enhanced down regulation of IDH1 in U87 cell line.

FIG. 8 shows that CaCl₂) salted PLGA SNAs showed enhanced down regulation of HER2 in SKOV-3 cell line.

FIG. 9 shows results of a Picogreen assay demonstrating that Ca²⁺ Ions are associated within the siRNA duplexes of the PLGA SNA. Picogreen exclusion assay of PLGA SNA and CaCl₂) salted PLGA SNA without 40 mM EDTA in picogreen solution (light purple) and with 40 mM EDTA in picogreen solution. Error bars are standard deviation of four independent measurements. ns: not significant, **** p<0.001

FIG. 10 shows a synthesis scheme and characterization of CaCl₂)-salted PLGA SNAs. (A) PLGA particles were formed via nanoprecipitation with incorporation of PLGA-PEG5000-N₃. DBCO-modified siRNA duplexes were immobilized on the PLGA particles by copper-free click chemistry and concomitant salting with either NaCl or CaCl₂) to form NaCl-salted PLGA SNAs (PLGA-SNAs) or CaCl₂)-salted PLGA SNAs (CaCl₂) PLGA SNAs), respectively. (B) DLS of the PLGA particles, PLGA SNAs and CaCl₂) PLGA SNAs. The diameter shown is the number mean. (C) Zeta potential of the PLGA particles, PLGA SNAs, and CaCl₂) PLGA SNAs (D) Fluorescence measurement (λ_{excitation}=480 nm, λₑₘᵢₛₛᵢₒₙ=520 nm) from PicoGreen exclusion assay of PLGA SNAs, CaCl₂) PLGA SNAs and CaCl₂)-siRNAs treated with PicoGreen solution and with 40 mM EDTA in PicoGreen solution. (E) Percentage of Ca²⁺ ion associated within CaCl₂) PLGA SNAs and CaCl₂)-siRNA complexes at multiple time points after dialysis. The percentage of Ca²⁺ remaining was determined by Picogreen exclusion assay. Error bars are the SDs of three independent measurements. ns: not significant, * p<0.05, ** p<0.01, **** p<0.0001. Only key significances are shown for clarity.

FIG. 11 shows cellular uptake of PLGA SNAs and CaCl₂) PLGA SNAs in U87-MG cells. All cells were treated at an Cy5 labeled siRNA concentration of 100 nM for 1 hour and the fold change in median fluorescence intensity (MFI) of each treatment was normalized to the MFI of the untreated cells. (A) Comparison of the cellular uptake of linear siRNAs and PLGA SNAs. (B) Comparison of the cellular uptake of CaCl₂)-siRNAs and CaCl₂) PLGA SNAs (salted to 230 mM CaCl₂)). (C) Effect of inhibitors on the uptake of PLGA SNAs. (D) Effect of inhibitors on the uptake of CaCl₂) PLGA SNAs. Fucoidan concentration: 50 μg/mL, Nifedipine concentration: 20 μM. The error bars are the SD of three independent measurements. ns: not significant, ** p<0.01, **** p<0.0001. Note the substantial difference in y-axis scales.

FIG. 12 shows intracellular trafficking analysis of PLGA SNAs and CaCl₂) PLGA SNAs in U87-MG cells via confocal microscopy. (A) Comparison of the cellular uptake of PLGA SNAs and CaCl₂) PLGA SNAs after 24 hour treatment at an siRNA concentration of 100 nM. Fold change in MFI of each treatment was normalized to the MFI of the untreated cells. (B) Representative confocal microscopy images showing Cy5-siRNA colocalization with late endosomes (Rab7a-GFP fusion protein) when treated with PLGA SNAs or CaCl₂) PLGA SNAs (salted to 230 mM CaCl₂)). Areas of colocalization appear yellow in the merged image. (Scale bars=5 μm) (C) Quantitative analysis of colocalization of Cy5-siRNA with Rab7a-GFP fusion protein by Mander's overlap coefficient (MOC). The error bars are the SD of ten independent measurements. **** p<0.0001.

FIG. 13 shows that CaCl₂) PLGA SNAs showed enhanced gene regulation of the Luc2 gene in U87-MG-Luc2 reporter cell line. (A) Knockdown potency of CaCl₂) PLGA SNAs after 48 hour treatment salted to different CaCl₂) concentrations. CaCl₂) PLGA SNAs were administered at an siRNA concentration of 100 nM. (B) Cell viability of U87-MG-Luc2 cells after 48 hour treatment of Luc2 targeting CaCl₂) PLGA SNAs salted to different CaCl₂) concentrations. (C) Comparison of Luc2 knockdown activity of CaCl₂) PLGA SNAs (salted to 230 mM CaCl₂)). The final siRNA concentration treated to the cells across treatment groups was 100 nM (D) Effect of endosomal acidification inhibition on CaCl₂) PLGA SNAs. The final siRNA concentration treated to the cells across treatment groups was 100 nM. The concentration of bafilomycin A1 pretreated to the U87-MG-Luc2 cells was 200 nM. The error bars are SD of three independent measurements. ns: not significant, * p<0.05, ** p<0.01, **** p<0.0001.

FIG. 14 demonstrates that CaCl₂) PLGA SNAs enhanced gene silencing activity of IDH1 in U87-MG cells and HER2 in SK-OV-3 cells. (A) Representative Western blot showing the knockdown potency of CaCl₂) PLGA SNA targeting IDH1 after 48 hour treatment. HSP70 served as a loading control. The band intensity was normalized to HSP70 and then quantified as relative expression compared to the untreated control. The final siRNA concentration treated to the cells across the treatment groups was 100 nM, and the CaCl₂) concentration salted to CaCl₂) PLGA SNA was 230 mM. (B) IDH1 densiometric analysis of Western blots. The error bars are the SDs of three independent experiments. (C) Comparison of HER2 knockdown activity of CaCl₂) PLGA SNAs in SK-OV-3 cell line. HER2 expression was quantified by an in-cell Western assay. The final siRNA concentration treated to the cells across the treatment groups was 100 nM, and the CaCl₂) concentration salted to CaCl₂)-salted PLGA SNA was 230 mM. (D) Cell viability of SK-OV-3 cells after 48 hour treatment of HER2-targeting CaCl₂) PLGA SNAs salted to different CaCl₂) concentrations. Cell viability was measured using a CellTag 700 stain during in-cell Western Blot assay. The error bars are the SDs of three independent measurements. ns: not significant, * p<0.05, **** p<0.0001. Only key significances are shown for clarity.

FIG. 15 shows Dynamic Light Scattering (DLS) size distribution of PLGA particles, PLGA SNAs (NaCl salted) and CaCl₂)-salted PLGA SNAs (CaCl₂) PLGA SNAs). The size increase from the bare PLGA particles to the PLGA SNAs and CaCl₂) PLGA SNAS indicates functionalization of siRNA to the PLGA particle surface. Moreover, these data do not indicate that significant aggregation and polydispersity occurs when CaCl₂) is used. The error bars are the standard deviation (SD) of three independent measurements.

FIG. 16 shows siRNA duplex loading and surface loading density of PLGA SNAs and CaCl₂) PLGA SNAs. The counterion used during salting (Na⁺ or Ca²⁺) did not affect the siRNA loading and the surface density of the PLGA SNAs. The error bars are the SDs of three independent measurements.

FIG. 17 shows zeta potential of the PLGA particles, PLGA SNAs, and CaCl₂) PLGA SNAs salted with different CaCl₂) concentrations. The error bars are the SDs of three independent measurements. ** p<0.01, **** p<0.0001.

FIG. 18 shows that Luc2-targeting siRNA down regulated Luc2 protein expression in the U87-MG-Luc2 cell line when transfected with lipofectamine RNAiMAX. [siRNA]=100 nM. The error bars are the SDs of three independent measurements. **** p<0.0001.

FIG. 19 shows that CaCl₂) PLGA SNAs downregulated Luc2 expression at the mRNA level. Comparison of the Luc2 knockdown activity of CaCl₂) PLGA SNAs (salted to 230 mM CaCl₂)). The final siRNA concentration treated to the cells for all treatment groups was 100 nM. After a 48-hour treatment, relative Luc2 mRNA expression levels were analyzed by RT-qPCR. The error bars represent the SDs of three independent measurements. ** p<0.01, **** p<0.0001.

FIG. 20 shows Luc2 Antisense DNA functionalized CaCl₂) PLGA SNAs down regulated Luc2 expression at the protein level. Comparison of Luc2 knockdown activity of antisense DNA functionalized CaCl₂) PLGA SNAs in U87-MG-Luc2 cell line. The DNA concentration treated to the cells across all treatment groups is 1 μM, and the CaCl₂) concentration the PLGA SNA that was salted in was 333 mM. After a 48-hour treatment, Luc2 expression was quantified by luminescence assay. The error bars are the SDs of three independent measurements. ** p<0.01, **** p<0.0001. Not all significances are shown for clarity.

FIG. 21 shows that CaCl₂) PLGA SNAs functionalized with thiazole orange incorporated poly T21 DNA strands exhibited enhanced fluorescence, indicating cytosolic detection of poly A tail of mRNAs. (A) Mechanism of thiazole orange (TO) incorporated DNA probes. Upon hybridization to a complementary sequence, TO intercalates within the formed duplex and exhibits enhanced fluorescence. (B) Fluorescence response of T21/A21 linear flare strands, PLGA SNAs and CaCl₂) PLGA SNAs to increased concentrations of complementary poly A21 RNA. T21 flare strand exhibits 15-fold enhancement in presence of 1 molar equivalence of complementary poly A21 RNA. The amount of flare strand to determine fluorescent turn-on was 2.5 pmol. (C) Representative live confocal microscopy images of U87-MG-Luc2 cells with different treatment conditions after 2-hours. The CaCl₂) concentration for CaCl₂) PLGA SNA was 333 mM and 20 pmole (by DNA concentration) was treated to the cells. Scale bar: 10 μm. (D) Mean pixel intensity of TO signal from each treatment conditions (mean±SD, n=20, except linear A21 flare transfected with lipofectamine 2000, n=10). Statistical significance was determined by one way ANOVA followed by post-hoc Tukey multiple comparison test. sig **** p<0.0001. Not all significances are shown for clarity. (E) Fluorescence response of T21/A21 linear flare strands, PLGA SNAs and CaCl₂) PLGA SNAs to varying amount RNA extract from U87-MG-Luc2 cells. The amount of flare strand to determine fluorescent turn-on was 2.5 pmole. From the amount of RNA extract that corresponds to 4,000 to 150,000 cells, maximum of 5.6-fold turn-on was achieved.

FIG. 22 shows that CaCl₂) PLGA SNAs down regulated HER2 expression at the protein level. Comparison of HER2 knockdown activity of CaCl₂)-salted PLGA SNAs in SK-OV-3 cell line. HER2 expression was quantified using an in-cell Western Blot assay. The final siRNA concentration treated to the cells across all treatment groups is 100 nM, and the CaCl₂) concentration the PLGA SNAs were salted in 230 mM, 290 mM, and 333 mM. After a 48-hour treatment, HER2 expression was quantified by in-cell Western Blot assay. The error bars are the SDs of three independent measurements. **** p<0.0001. Not all significances are shown for clarity.

### DETAILED DESCRIPTION

Gene regulation therapy with inhibitor oligonucleotides (e.g., siRNAs) is a promising alternative to traditional small molecule or protein-based therapeutics.¹ Inhibitory oligonucleotides can be designed to specifically degrade complementary target mRNA upon binding to the RNA-induced silencing complex (RISC).^{2,3} Thus, RNA interference (RNAi) is a promising technology in which inhibitory oligonucleotides (e.g., small interfering RNAs (siRNAs)) can be designed to silence any target gene, exhibiting potential for treating diseases that are regarded as “undruggable” by conventional medicines. While inhibitor oligonucleotides such as siRNAs show therapeutic potential, they do not have the ability to readily enter cells on their own in part due to their negatively charged phosphate backbone, and they are susceptible to rapid degradation by nucleases, making it a challenge to broadly use them in clinical settings.^{4,5}

Inhibitory oligonucleotide (e.g., siRNA) conjugated spherical nucleic acids (SNAs), where inhibitory oligonucleotides are densely packed and radially oriented around a nanoparticle core into a spherical architecture provide distinct properties with enhanced cellular uptake, resistance to nuclease degradation without eliciting non-specific immune response.^{6,7} The SNA architecture has unique structure-dependent properties that can be exploited to overcome the challenges associated with the delivery of inhibitory oligonucleotides such as linear siRNA or antisense DNA. The three-dimensional oligonucleotide shell bestows SNAs with the ability to enter cells via scavenger receptor A-mediated endocytosis,^{8,9} and the dense shell that defines the SNA minimizes nuclease degradation.¹⁰ When siRNA-based SNAs enter the cytosol, they associate with the RISC complex to mediate gene silencing; thus, SNAs are promising entities for gene regulation therapeutics and have been used in hundreds of cell based knockdown experiments (e.g., EGFP11,12, GAPDH13, LUC214-17, EGFR18-20, Bcl2L1221,22, BcL223,24, GM3S25, HER226-28, MGMT29, Survivin20, MFG-E830, PLK112, IL17RA31, TGFβ32, STAT333, TIMP134, TNF-α34 and PDL135) and multiple human clinical trials.^{22,36,37} However, some decreased gene regulation efficiency of siRNA functionalized SNAs has been observed due to sequence dependent and cell-line dependent differences in the cytosolic delivery of the siRNA-SNA construct to associate with the RNA-induced silencing complex (RISC) for gene regulation. To realize potent gene regulatory activity, SNAs must be delivered to the cytosol to access the RISC complex and target mRNA, as is the case with all gene silencing therapies.^{38,39} The uptake pathway for SNAs involves trafficking through the endosomal pathway with accumulation in the late endosome, while only a small portion of the SNAs escape to the cytosol where they can engage in gene silencing.⁴⁰ The extent of escape and corresponding activity are highly dependent upon sequence, cell type, and perhaps even cell cycle, making the identification of a lead structure for a given target a difficult-to-predict process. To improve the potency and increase the generality of siRNA-SNA based gene regulation constructs, a method that improves the cytosolic delivery of SNAs more broadly needs to be established and would be highly beneficial for biological and medical applications involving SNAs.

Hence, to improve inhibitory oligonucleotide-SNA's gene regulation efficiency, the present disclosure provides calcium chloride (CaCl₂)) salted SNAs (e.g., poly-lactic-co-glycolic acid (PLGA) SNAs) where Ca²⁺ ions are bound to the phosphate backbone of the oligonucleotide shell. As shown in the Examples herein, CaCl₂) salted PLGA SNAs did not show any particle aggregation and exhibited increased zeta potential compared to regular PLGA SNAs (not salted with CaCl₂)). When treated to cells, CaCl₂) salted PLGA SNAs exhibited significantly improved gene regulation efficiency compared to regular PLGA SNAs by almost 20-fold independent of the siRNA sequence functionalized to the SNA surface without eliciting any cellular toxicity.

As used in this specification and the appended claims, the singular forms “a,” “an,” and “the” include plural reference unless the context clearly dictates otherwise.

The terms “polynucleotide” and “oligonucleotide” are interchangeable as used herein.

As used herein, the term “about,” when used to modify a particular value or range, generally means within 20 percent, e.g., within 10 percent, 5 percent, 4 percent, 3 percent, 2 percent, or 1 percent of the stated value or range.

Unless otherwise stated, all ranges contemplated herein include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

A “subject” is a vertebrate organism. The subject can be a non-human mammal (e.g., a mouse, a rat, or a non-human primate), or the subject can be a human subject.

The terms “administering”, “administer”, “administration”, and the like, as used herein, refer to any mode of transferring, delivering, introducing, or transporting a SNA to a subject in need of treatment with such an agent. Such modes include, but are not limited to, oral, topical, intravenous, intraarterial, intraperitoneal, intramuscular, intratumoral, intradermal, intranasal, and subcutaneous administration.

As used herein, “treating” and “treatment” refers to any reduction in the severity and/or onset of symptoms associated with a disease (e.g., cancer). Accordingly, “treating” and “treatment” includes therapeutic and prophylactic measures. One of ordinary skill in the art will appreciate that any degree of protection from, or amelioration of, the disease (e.g., cancer) is beneficial to a subject, such as a human patient. The quality of life of a patient is improved by reducing to any degree the severity of symptoms in a subject and/or delaying the appearance of symptoms.

As used herein, a “targeting oligonucleotide” is an oligonucleotide that directs a SNA to a particular tissue and/or to a particular cell type or it is an oligonucleotide that detects a target analyte. In some embodiments, a targeting oligonucleotide is an aptamer. Thus, in some embodiments, a SNA of the disclosure comprises an aptamer attached to the exterior of the nanoparticle core, wherein the aptamer is designed to bind one or more receptors on the surface of a certain cell type, or the aptamer is designed to detect a target analyte. Target analytes contemplated by the disclosure include without limitation a protein, an ion, a small molecule, a lipid, a carbohydrate, an oligosaccharide, a cell, an oligonucleotide, or a combination thereof.

As used herein, an “immunostimulatory oligonucleotide” is an oligonucleotide that can stimulate (e.g., induce or enhance) an immune response. Typical examples of immunostimulatory oligonucleotides are CpG-motif containing oligonucleotides, single-stranded RNA oligonucleotides, double-stranded RNA oligonucleotides, and double-stranded DNA oligonucleotides. A “CpG-motif” is a cytosine-guanine dinucleotide sequence. In any of the aspects or embodiments of the disclosure, the immunostimulatory oligonucleotide is a toll-like receptor (TLR) agonist (e.g., a toll-like receptor 9 (TLR9) agonist). In various embodiments, about or at least about 5%, 10%, 20%, 30%, 40%, 50%, 60%, 70% 80%, 90%, 95%, or 100% of oligonucleotides in the shell of oligonucleotides of a SNA as described herein are immunostimulatory oligonucleotides.

The term “inhibitory oligonucleotide” refers to an oligonucleotide that reduces the production or expression of proteins, such as by interfering with translating mRNA into proteins in a ribosome or that are sufficiently complementary to either a gene or an mRNA encoding one or more of targeted proteins, that specifically bind to (hybridize with) the one or more targeted genes or mRNA thereby reducing expression or biological activity of the target protein. Inhibitory oligonucleotides include, without limitation, isolated or synthetic short hairpin RNA (shRNA or DNA), an antisense oligonucleotide (e.g., antisense RNA or DNA, chimeric antisense DNA or RNA), miRNA and miRNA mimics, small interfering RNA (siRNA), DNA or RNA inhibitors of innate immune receptors, an aptamer, a DNAzyme, or an aptazyme.

An “effective amount” or a “sufficient amount” of a substance is that amount necessary to effect beneficial or desired results, including clinical results, and, as such, an “effective amount” depends upon the context in which it is being applied. In the context of administering a SNA of the disclosure, for example, an effective amount is an amount sufficient to inhibit gene expression. Efficacy can be shown in an experimental or clinical trial, for example, by comparing results achieved with a substance of interest compared to an experimental control.

All references, patents, and patent applications disclosed herein are incorporated by reference with respect to the subject matter for which each is cited, which in some cases may encompass the entirety of the document.

### Spherical Nucleic Acids (SNAs)

The present disclosure provides calcium salted spherical nucleic acids (SNAs) and compositions comprising calcium salted SNAs. As described herein above, calcium chloride (CaCl₂)) salted SNAs of the disclosure comprise Ca²⁺ ions that are bound to one or more oligonucleotides (e.g., the phosphate backbone and/or the nucleobase) of the oligonucleotide shell. Calcium (Ca²⁺) ions can not only adsorb (bind) to the phosphate backbone of an oligonucleotide, but can also bind to the nucleobases, N7 and O6 atoms on guanine (G), N7 atom on adenine (A), O2 atom on cytosine (C) and O4 atom on uracil (U) or thymine (T) (see, e.g., J. Phys. Chem. B 2022, 126, 43, 8646-8654, Acc. Chem. Res. 2010, 43, 7, 974-984, and Langmuir, (2020), 5979-5989, 36 (21)). Thus, possible binding sites for Ca²⁺ on an oligonucleotide include both negatively charged phosphate oxygens of the phosphate backbone and the nitrogens and/or oxygens on the nucleobases. The disclosure therefore contemplates that in various embodiments there a SNA of the disclosure comprises or consists of between 1 to 3 Ca²⁺ ions per nucleotide (each phosphate backbone has one Ca²⁺ binding site while the nucleobases have additional binding sites). The amount of Ca²⁺ ions adsorbed to a SNA of the disclosure may also be expressed as a percentage of the total available Ca²⁺ binding sites on a SNA that are occupied by a Ca²⁺ ion. In various embodiments, about, at least about, or less than about 5%, about, at least about, or less than about 10%, about, at least about, or less than about 15%, about, at least about, or less than about 20%, about, at least about, or less than about 25%, about, at least about, or less than about 30%, about, at least about, or less than about 35%, about, at least about, or less than about 40%, about, at least about, or less than about 45%, about, at least about, or less than about 50%, about, at least about, or less than about 55%, about, at least about, or less than about 60%, about, at least about, or less than about 65%, about, at least about, or less than about 70%, about, at least about, or less than about 75%, about, at least about, or less than about 80%, about, at least about, or less than about 85%, about, at least about, or less than about 90%, about, at least about, or less than about 95%, or about or less than about 100% of the total available Ca²⁺ binding sites on a SNA are occupied by a Ca²⁺ ion. As taught herein (see, e.g., Example 2), to verify the association of Ca²⁺ ions in a SNA of the disclosure (a CaCl₂) salted SNA), one can conduct an exclusion assay using an intercalating dye. Intercalating dyes that may be used include, but are not limited to, Picogreen™ (Thermo Fisher Scientific Inc., Waltham, MA), ethidium bromide, thiazole orange (TO), SYBR green, and LAMP Fluorescent Dye (New England Biolabs Inc., Ipswich, MA).

Electrostatic adsorption of Ca²⁺ ions to the oligonucleotides in the shell of oligonucleotides of a SNA would physically screen the oligonucleotide shell, preventing the dye from intercalating within the oligonucleotide and consequently would lead to a decrease in intercalating dye fluorescence intensity. However, a divalent cation chelator (e.g., ethylenediaminetetraacetic acid (EDTA)) would chelate the Ca²⁺ ions and enable the intercalation of the dye within the oligonucleotide and lead to an increase in fluorescence intensity. Thus, in various embodiments, a SNA of the disclosure has an intercalating dye intensity that is decreased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to intercalating dye intensity of a NaCl-salted SNA under identical conditions. SNAs comprise a nanoparticle core surrounded by a shell of oligonucleotides. In various embodiments, an oligonucleotide shell is formed when at least 10% of the available surface area of the exterior surface of a nanoparticle core includes an oligonucleotide. In further embodiments at least 20%, 30%, 40%, 50%, 60%, 70%, 80%, 90%, 95%, 96%, 97%, 98% or 99% of the available surface area of the exterior surface of the nanoparticle core includes an oligonucleotide. The oligonucleotides of the oligonucleotide shell may be oriented in a variety of directions. In some embodiments the oligonucleotides are oriented radially outwards. The oligonucleotide shell comprises one or more oligonucleotides attached to the external surface of the nanoparticle core. The spherical architecture of the polynucleotide shell confers unique advantages over traditional nucleic acid delivery methods, including entry into nearly all cells independent of transfection agents and resistance to nuclease degradation. Furthermore, SNAs can penetrate biological barriers, including the blood-brain (see, e.g., U.S. Patent Application Publication No. 2015/0031745, incorporated by reference herein in its entirety) and blood-tumor barriers as well as the epidermis (see, e.g., U.S. Patent Application Publication No. 2010/0233270, incorporated by reference herein in its entirety).

Thus, in any of the aspects or embodiments of the disclosure, a spherical nucleic acid (SNA) is provided comprising (a) a nanoparticle core; and (b) a shell of oligonucleotides attached to the external surface of the nanoparticle core, wherein one or more oligonucleotides in the shell of oligonucleotides comprises a phosphate backbone; the SNA comprising Ca²⁺ ions adsorbed to the phosphate backbone of one or more oligonucleotides in the shell of oligonucleotides. In some embodiments, each oligonucleotide in the shell of oligonucleotides comprises a phosphate backbone. In some embodiments, Ca²⁺ ions are adsorbed to the phosphate backbone of each oligonucleotide in the shell of oligonucleotides. In further aspects, the disclosure provides a spherical nucleic acid (SNA) comprising: (a) a nanoparticle core; and (b) a shell of oligonucleotides attached to the external surface of the nanoparticle core, wherein one or more oligonucleotides in the shell of oligonucleotides comprises a phosphate backbone; the SNA comprising Ca²⁺ ions adsorbed to one or more oligonucleotides in the shell of oligonucleotides. In some embodiments, each oligonucleotide in the shell of oligonucleotides comprises a phosphate backbone. In further embodiments, Ca²⁺ ions are adsorbed to the phosphate backbone of one or more oligonucleotides in the shell of oligonucleotides. In various embodiments, Ca²⁺ ions are adsorbed to the phosphate backbone of each oligonucleotide in the shell of oligonucleotides. In any of the aspects or embodiments of the disclosure, Ca²⁺ ions are adsorbed to one or more bases of one or more oligonucleotides in the shell of oligonucleotides. In some embodiments, the SNA has a zeta potential that is about −40 millivolts (mV) to about −10 mV, or about −20 mV to about −10 mV, or about −15 mV to about −10 mV. In further embodiments, the SNA has a zeta potential that is about −30 mV, −20, mV, −15 mV, or about −10 mV.

SNAs can range in size from about 1 nanometer (nm) to about 1000 nm, about 1 nm to about 900 nm, about 1 nm to about 800 nm, about 1 nm to about 700 nm, about 1 nm to about 600 nm, about 1 nm to about 500 nm, about 1 nm to about 400 nm, about 1 nm to about 300 nm, about 1 nm to about 200 nm, about 1 nm to about 150 nm, about 1 nm to about 100 nm, about 1 nm to about 90 nm, about 1 nm to about 80 nm in diameter, about 1 nm to about 70 nm in diameter, about 1 nm to about 60 nm in diameter, about 1 nm to about 50 nm in diameter, about 1 nm to about 40 nm in diameter, about 1 nm to about 30 nm in diameter, about 1 nm to about 20 nm in diameter, about 1 nm to about 10 nm, about 10 nm to about 150 nm in diameter, about 10 nm to about 140 nm in diameter, about 10 nm to about 130 nm in diameter, about 10 nm to about 120 nm in diameter, about 10 nm to about 110 nm in diameter, about 10 nm to about 100 nm in diameter, about 10 nm to about 90 nm in diameter, about 10 nm to about 80 nm in diameter, about 10 nm to about 70 nm in diameter, about 10 nm to about 60 nm in diameter, about 10 nm to about 50 nm in diameter, about 10 nm to about 40 nm in diameter, about 10 nm to about 30 nm in diameter, or about 10 nm to about 20 nm in diameter. In further embodiments, the SNA is, is at least, or is less than about 1000 nm, 900 nm, 800 nm, 700 nm, 600 nm, 500 nm, 400 nm, 300 nm, 200 nm, 100 nm, 90 nm, 80 nm, 70 nm, 60 nm, 50 nm, 40 nm, 30 nm, 20, or 10 nm in diameter (or in mean diameter when there are a plurality of SNAs). In further aspects, the disclosure provides a plurality of SNAs, each SNA comprising an oligonucleotide shell attached to the external surface of the nanoparticle core, wherein each oligonucleotide in the shell of oligonucleotides comprises a phosphate backbone; the SNA comprising Ca²⁺ ions adsorbed to the phosphate backbone of one or more oligonucleotides in the shell of oligonucleotides. Thus, in some embodiments, the size of the plurality of SNAs is from about 10 nm to about 1000 nm (mean diameter), about 10 to about 900 nm in mean diameter, about 10 to about 800 nm in mean diameter, about 10 to about 700 nm in mean diameter, about 10 to about 600 nm in mean diameter, about 10 to about 500 nm in mean diameter, about 10 to about 400 nm in mean diameter, about 10 to about 300 nm in mean diameter, about 10 to about 200 nm in mean diameter, about 10 to about 150 nm (mean diameter), about 10 nm to about 140 nm in mean diameter, about 10 nm to about 130 nm in mean diameter, about 10 nm to about 120 nm in mean diameter, about 10 nm to about 110 nm in mean diameter, about 10 nm to about 100 nm in mean diameter, about 10 nm to about 90 nm in mean diameter, about 10 nm to about 80 nm in mean diameter, about 10 nm to about 70 nm in mean diameter, about 10 nm to about 60 nm in mean diameter, about 10 nm to about 50 nm in mean diameter, about 10 nm to about 40 nm in mean diameter, about 10 nm to about 30 nm in mean diameter, or about 10 nm to about 20 nm in mean diameter. In some embodiments, the diameter (or mean diameter for a plurality of SNAs) of the SNAs is from about 10 nm to about 150 nm, from about 30 to about 100 nm, or from about 40 to about 80 nm. In some embodiments, the size of the nanoparticles used in a method varies as required by their particular use or application. The variation of size is advantageously used to optimize certain physical characteristics of the SNAs, for example, the amount of surface area to which oligonucleotides may be attached as described herein. It will be understood that the foregoing diameters of SNAs can apply to the diameter of the nanoparticle core itself or to the diameter of the nanoparticle core and the oligonucleotide shell attached thereto. Further description of nanoparticle cores is provided herein below.

In various embodiments, the nanoparticle core is a metallic core, a semiconductor core, an insulator core, an upconverting core, a micellar core, a dendrimer core, a liposomal core, a lipid nanoparticle core, a polymer core, a metal-organic framework core, a protein core, or a combination thereof. In various embodiments, the polymer is polylactide, a polylactide-polyglycolide copolymer, a polycaprolactone, a polyacrylate, alginate, albumin, polypyrrole, polythiophene, polyaniline, polyethylenimine, poly(methyl methacrylate), poly(lactic-co-glycolic acid) (PLGA), or chitosan.

PLGA-SNAs may be synthesized using several strategies. In some embodiments, a PLGA SNA is synthesized by conjugating lipid-modified oligonucleotides to the surface of PLGA nanoparticles via hydrophobic-hydrophobic interactions. In some embodiments, a PLGA SNA is synthesized by conjugating oligonucleotide and the PLGA, which comprise complementary reactive moieties that together form a covalent bond. In some embodiments, DBCO-modified DNA strands are covalently conjugated to, e.g., azide groups through Cu-free click chemistry [Baskin, et al. Proc. Natl. Acad. Sci. U.S.A. 2007, 104 (43), 16793-16797]. While DBCO-modified oligonucleotides were used in examples herein, other alkyne moieties can be used instead, including a terminal alkyne (HC≡C—) or an internal alkyne (RC≡C—, where R comprises an alkyl). The alkyne moiety can also be attached to the oligonucleotide via a linker. In some embodiments, the reactive moiety on the nanoparticle core (e.g., a polymer comprising PLGA or in some embodiments PLGA-PEG) comprises an azide, an alkyne, a maleimide, a thiol, an alcohol, an amine, a carboxylic acid, an olefin, an isothiocyanate, a N-hydroxysuccinimide, a phosphine, a nitrone, a norbornene, an oxanorbornene, a transcycloctene, an s-tetrazene, an isocyanide, a tetrazole, a nitrile oxide, a quadricyclane, or a carbodiimide. In further embodiments, the reactive moiety on the oligonucleotide is on a terminus of the oligonucleotide. In still further embodiments, the reactive moiety on the oligonucleotide comprises an alkyne, an azide, a maleimide, a thiol, an alcohol, an amine, a carboxylic acid, an olefin, an isothiocyanate, a N-hydroxysuccinimide, a phosphine, a nitrone, a norbornene, an oxanorbornene, a transcycloctene, an s-tetrazene, an isocyanide, a tetrazole, a nitrile oxide, a quadricyclane, or a carbodiimide. In some embodiments, the alkyne comprises dibenzocyclooctyl (DBCO) alkyne or a terminal alkyne. In further embodiments, the polymer (e.g., PLGA or PLGA-PEG) comprises an azide reactive moiety and the oligonucleotide comprises an alkyne reactive moiety, or vice versa. In still further embodiments, the alkyne reactive moiety comprises a DBCO alkyne. The PLGA-SNAs of the disclosure may contain a polymer selected from the group consisting of diblock poly(lactic) acid-poly(ethylene)glycol (PLA-PEG) copolymer, diblock poly(lactic acid-co-glycolic acid)-poly(ethylene)glycol (PLGA-PEG) copolymer, and combinations thereof. PLGA-SNAs are further described herein below and in International Publication No. WO 2018/175445, which is incorporated by reference herein in its entirety.

In some embodiments, the nanoparticle core is gold, silver, platinum, aluminum, palladium, copper, cobalt, indium, cadmium selenide, iron oxide, fullerene, metal-organic framework, silica, zinc sulfide, or nickel. In some embodiments, the nanoparticle core is a liposome. In further embodiments, the liposome comprises a lipid selected from the group consisting of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), and cholesterol.

Liposomes are spherical, self-closed structures in a varying size range comprising one or several hydrophobic lipid bilayers with a hydrophilic core. The diameter of these lipid based carriers range from 0.15-1 micrometers, which is significantly higher than an effective therapeutic range of 20-100 nanometers. Liposomes termed small unilamellar vesicles (SUVs), can be synthesized in the 20-50 nanometer size range, but encounter challenges such as instability and aggregation leading to inter-particle fusion. This inter-particle fusion limits the use of SUVs in therapeutics. In some aspects, liposomal spherical nucleic acids (LSNAs) comprise a liposomal core, a shell of oligonucleotides attached to the external surface of the liposomal core, the shell of oligonucleotides comprising one or more oligonucleotides comprising a phosphate backbone; the SNA comprising Ca²⁺ ions adsorbed to the phosphate backbone of one or more oligonucleotides in the shell of oligonucleotides.

In various embodiments, one or more oligonucleotides in the shell of oligonucleotides is attached to the external surface of the nanoparticle core (e.g., liposomal core) through a lipid anchor group. In some embodiments, each oligonucleotide in the shell of oligonucleotides is attached to the external surface of the nanoparticle core through a lipid anchor group. In further embodiments, the lipid anchor group is attached to the 5′ end or the 3′ end of the at least one oligonucleotide. In still further embodiments, the lipid anchor group is tocopherol or cholesterol. Thus, in various embodiments, at least one of the oligonucleotides in the shell of oligonucleotides is an oligonucleotide-lipid conjugate containing a lipid anchor group, wherein said lipid anchor group is adsorbed into the lipid bilayer. In some embodiments, all of the oligonucleotides in the shell of oligonucleotides is an oligonucleotide-lipid conjugate containing a lipid anchor group, wherein said lipid anchor group is adsorbed into the lipid bilayer. In various embodiments, 10%, 20%, 30%, 40%, 50%, 60%, 70%, 80%, 90% or 100% of the oligonucleotides in the shell of oligonucleotides is attached (e.g., adsorbed) to the exterior of the liposomal core through a lipid anchor group. The lipid anchor group comprises, in various embodiments, tocopherol, palmitoyl, dipalmitoyl, stearyl, distearyl, or cholesterol. While not meant to be limiting, any chemistry known to one of skill in the art can be used to attach the lipid anchor to the oligonucleotide, including amide linking or click chemistry.

Methods of making a liposomal SNA (LSNA) are described herein and are also described in, e.g., U.S. Pat. No. 10,182,988, incorporated by reference herein in its entirety.

Lipid nanoparticle spherical nucleic acids (LNP-SNAs) are comprised of a lipid nanoparticle core decorated with a shell of oligonucleotides. The lipid nanoparticle core comprises an ionizable lipid, a phospholipid, a sterol, and a lipid-polyethylene glycol (lipid-PEG) conjugate. The shell of oligonucleotides is attached to the external surface of the lipid nanoparticle core. The spherical architecture of the oligonucleotide shell confers unique advantages over traditional nucleic acid delivery methods, including entry into nearly all cells independent of transfection agents, resistance to nuclease degradation, sequence-based function, targeting, and diagnostics.

In some embodiments, the ionizable lipid is dilinoleylmethyl-4-dimethylaminobutyrate (DLin-MC3-DMA), 2,2-Dilinoleyl-4-dimethylaminoethyl-[1,3]-dioxolane (DLin-KC2-DMA), C12-200, 1,2-dioleoyl-3-dimethylammonium-propane (DODAP), similar lipid/lipidoid structures, or a combination thereof. In some embodiments, the phospholipid is 1,2-distearoyl-sn-glycero-3-phosphocholine (DSPC), 1,2-Dihexadecanoyl phosphatidylcholine (DPPC), 1,2-dioleoyl-sn-glycero-3-phosphocholine (DOPC), 1,2-dioleoyl-sn-glycero-3-phosphoethanolamine (DOPE), monophosphoryl Lipid A (MPLA), or a combination thereof. In further embodiments, the sterol is 3β-Hydroxycholest-5-ene (Cholesterol), 9,10-Secocholesta-5,7,10 (19)-trien-3ß-ol (Vitamin D3), 9,10-Secoergosta-5,7,10(19),22-tetraen-3β-ol (Vitamin D2), Calcipotriol, 24-Ethyl-5,22-cholestadien-3ß-ol (Stigmasterol), 22,23-Dihydrostigmasterol (β-Sitosterol), 3,28-Dihydroxy-lupeol (Betulin), Lupeol, Ursolic acid, Oleanolic acid, 24α-Methylcholesterol (Campesterol), 24-Ethylcholesta-5,24(28)E-dien-3β-ol (Fucosterol), 24-Methylcholesta-5,22-dien-3β-ol (Brassicasterol), 24-Methylcholesta-5,7,22-trien-3β-ol (Ergosterol), 9,11-Dehydroergosterol, Daucosterol, or any of the foregoing sterols modified with one or more amino acids. In some embodiments, the lipid-polyethylene glycol (lipid-PEG) conjugate comprises 2000 Dalton (Da) polyethylene glycol. In further embodiments, the lipid-polyethylene glycol (lipid-PEG) conjugate is lipid-PEG-maleimide. In still further embodiments, the lipid-PEG-maleimide is 1,2-dipalmitoryl-sn-glycero-3-phosphoethanolamine (DPPE) conjugated to 2000 Da polyethylene glycol maleimide, 1,2-dimyristoyl-sn-glycero-3-phosphoethanolamine (DMPE) conjugated to 2000 Da polyethylene glycol maleimide, or a combination thereof.

In any of the aspects or embodiments of the disclosure an oligonucleotide is attached to the external surface of a lipid nanoparticle core via a covalent attachment of the oligonucleotide to a lipid-polyethylene glycol (lipid-PEG) conjugate. In various embodiments, 10%, 20%, 30%, 40%, 50%, 60%, 70%, 80%, 90% or 100% of the oligonucleotides in the shell of oligonucleotides are covalently attached to the exterior of the lipid nanoparticle core through the lipid-PEG conjugate. In various embodiments, one or more oligonucleotides in the oligonucleotide shell is attached (e.g., adsorbed) to the exterior of the lipid nanoparticle core through a lipid anchor group as described herein. In various embodiments, 10%, 20%, 30%, 40%, 50%, 60%, 70%, 80%, 90% or 100% of the oligonucleotides in the shell of oligonucleotides is attached (e.g., adsorbed) to the exterior of the lipid nanoparticle core through a lipid anchor group as described herein. The lipid anchor group is, in various embodiments, attached to the 5′- or 3′-end of the oligonucleotide. In various embodiments, the lipid anchor group is tocopherol, palmitoyl, dipalmitoyl, stearyl, distearyl, or cholesterol.

### Methods of Making Calcium Salted SNAs

The disclosure also provides methods of making calcium salted SNAs. Accordingly, in some aspects the disclosure provides a method of making a calcium chloride (CaCl₂)) salted spherical nucleic acid (SNA), the SNA comprising: (a) a nanoparticle core; and (b) a shell of oligonucleotides attached to the external surface of the nanoparticle core, wherein one or more or each oligonucleotide in the shell of oligonucleotides comprises a phosphate backbone; the method comprising: combining the nanoparticle core, a plurality of oligonucleotides, and calcium chloride (CaCl₂)) to create a mixture, wherein the combining results in the plurality of oligonucleotides becoming attached to the nanoparticle core to create the shell of oligonucleotides attached to the external surface of the nanoparticle core, thereby resulting in the CaCl₂) salted SNA, and then optionally isolating the CaCl₂) salted SNA from the mixture. In some embodiments, Ca²⁺ ions are adsorbed to the phosphate backbone of one or more or each oligonucleotide in the shell of oligonucleotides of the CaCl₂) salted SNA. In some aspects or embodiments of the disclosure, Ca²⁺ ions are adsorbed to one or more bases of one or more oligonucleotides in the shell of oligonucleotides. By way of example, following the combining of the nanoparticle core, the plurality of oligonucleotides, and calcium chloride (CaCl₂)) to create a mixture, the mixture is incubated to allow the plurality of oligonucleotides to become attached to the external surface of the nanoparticle core. The incubating may be performed at room temperature for about 6-24 hours, and may include shaking. In various embodiments, the mixture comprises a nanoparticle core (e.g., PLGA), a plurality of oligonucleotides, a surfactant (e.g., Poloxamer 188), a salt (e.g., NaCl), and CaCl₂). Various concentrations of CaCl₂) may be utilized in the mixture. In various embodiments, the concentration of CaCl₂) in the mixture is, is about, is at least about, or is less than about 7 mM, 36 mM, 50 mM, 70 mM, 100 mM, 130 mM, 150 mM, 160 mM, 184 mM, 200 mM, 210 mM, 230 mM, 290 mM, 333 mM, or 350 mM CaCl₂). In some embodiments, the concentration of CaCl₂) in the mixture is about 70 mM to about 350 mM. In further embodiments, the concentration of CaCl₂) in the mixture is about 230 mM.

In some embodiments, the calcium salted SNA is produced using a nanoprecipitation method. The plurality of oligonucleotides may be attached to the nanoparticle core using any method(s) understood in the art and/or described herein. For example and without limitation, the oligonucleotides may be attached to the nanoparticle core via copper-free click chemistry. In some embodiments, the oligonucleotides comprise a lipid anchor group such that they can adsorb to the external surface of the nanoparticle core (e.g., a liposomal core). The oligonucleotides and the nanoparticle core may also comprise complementary reactive moieties that together form a covalent bond. The resulting calcium salted SNAs may be isolated by any method known in the art, for example and without limitation, spin filtration. General methods of making SNAs are also described herein above.

In various embodiments, calcium salted SNAs have a zeta potential that is about-40 millivolts (mV) to about −10 mV. In some embodiments, the CaCl₂) salted SNA has a zeta potential that is about −10 millivolts (mV). In further embodiments, the CaCl₂) salted SNA has a zeta potential that is, is about, is at least about, or is less than about −40 mV, −30 mV, −20 mV, −10 mV, or −5 mV. Zeta potential is measured, for example and without limitation, using a Zetasizer (e.g., Malvern Zetasizer Ultra Red).

### Oligonucleotides

The disclosure provides spherical nucleic acids (SNAs) comprising (a) a nanoparticle core; and (b) a shell of oligonucleotides attached to the external surface of the nanoparticle core, wherein one or more or each oligonucleotide in the shell of oligonucleotides comprises a phosphate backbone; the SNA comprising Ca²⁺ ions adsorbed to the phosphate backbone of one or more oligonucleotides in the shell of oligonucleotides. In various embodiments, the shell of oligonucleotides comprises an inhibitory oligonucleotide, an immunostimulatory oligonucleotide, a targeting oligonucleotide, or a combination thereof. Oligonucleotides contemplated for use according to the disclosure include those attached to a nanoparticle core through any means (e.g., covalent or non-covalent attachment). Oligonucleotides of the disclosure include, in various embodiments, DNA oligonucleotides, RNA oligonucleotides, modified forms thereof, or a combination thereof. In any aspects or embodiments described herein, an oligonucleotide is single-stranded, double-stranded, or partially double-stranded. In any aspects or embodiments of the disclosure, an oligonucleotide comprises a detectable marker. In various embodiments, the inhibitory oligonucleotide is an antisense oligonucleotide, small interfering RNA (siRNA), an aptamer, a short hairpin RNA (shRNA), a DNAzyme, or an aptazyme. In various embodiments, the immunostimulatory oligonucleotide comprises a CpG nucleotide sequence. In some embodiments, the immunostimulatory oligonucleotide is a toll-like receptor (TLR) agonist. In further embodiments, the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13). In some embodiments, the TLR is TLR9. In some embodiments, one or more oligonucleotides in the shell of oligonucleotides comprises or consists of the sequence of 5′-TCCATGACGTTCCTGACGTT-3′ (SEQ ID NO: 1). In further embodiments, one or more oligonucleotides in the shell of oligonucleotides comprises or consists of the sequence of 5′-TCGTCGTTTTGTCGTTTTGTCGTT-3′ (SEQ ID NO: 3). In still further embodiments, one or more oligonucleotides in the shell of oligonucleotides comprises or consists of the sequence of 5′-TCCATGACGTTCCTGACGTT (Spacer-18 (hexaethyleneglycol))₂Cholesterol-3′ (SEQ ID NO: 2). In still further embodiments, one or more oligonucleotides in the shell of oligonucleotides comprises or consists of the sequence of 5′-TCGTCGTTTTGTCGTTTTGTCGTT (Spacer-18 (hexaethyleneglycol))₂Cholesterol-3′ (SEQ ID NO: 4).

As described herein, modified forms of oligonucleotides are also contemplated by the disclosure which include those having at least one modified internucleotide linkage. In some embodiments, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function, e.g., as a probe in hybridization. Examples of universal bases include but are not limited to 5′-nitroindole-2′-deoxyriboside, 3-nitropyrrole, inosine and hypoxanthine.

The term “nucleotide” or its plural as used herein is interchangeable with modified forms as discussed herein and otherwise known in the art. The term “nucleobase” or its plural as used herein is interchangeable with modified forms as discussed herein and otherwise known in the art. Nucleotides or nucleobases comprise the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30:613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide”.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e., a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still further embodiments, oligonucleotide mimetics wherein both one or more sugar and/or one or more internucleotide linkage of the nucleotide units are replaced with “non-naturally occurring” groups. The bases of the oligonucleotide are maintained for hybridization. In some aspects, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In still further embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligonucleotide consists of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR″—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(O CH₂CH₃)—O—, —O—PO(O CH₂CH₂S—R)—O—, —O—PO(BH₃)—O—, —O—PO(NHR^{N})—O—, —O—P(O)₂—NR^{H} H—, —NR^{H}—P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—O—P(—O,S)—O—, —O—P(S)₂—O—, —NR^{H} P(O)₂—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl, are contemplated. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. patent application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O—, S—, or N-alkyl; O—, S—, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH2, heterocycloalkyl, heterocycloalkaryl, aminoalkylamino, polyalkylamino, substituted silyl, or an RNA cleaving group. In one aspect, a modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In some aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2^{nd} ed. 1989) and F. Eckstein (ed.) Oligonucleotides and Analogues, 1^{st} Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In various aspects, an oligonucleotide of the disclosure, or a modified form thereof, is generally about 5 nucleotides to about 1000 nucleotides in length. More specifically, an oligonucleotide of the disclosure is about 5 to about 1000 nucleotides in length, about 5 to about 900 nucleotides in length, about 5 to about 800 nucleotides in length, about 5 to about 700 nucleotides in length, about 5 to about 600 nucleotides in length, about 5 to about 500 nucleotides in length about 5 to about 450 nucleotides in length, about 5 to about 400 nucleotides in length, about 5 to about 350 nucleotides in length, about 5 to about 300 nucleotides in length, about 5 to about 250 nucleotides in length, about 5 to about 200 nucleotides in length, about 5 to about 150 nucleotides in length, about 5 to about 100, about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, about 10 to about 1000 nucleotides in length, about 10 to about 900 nucleotides in length, about 10 to about 800 nucleotides in length, about 10 to about 700 nucleotides in length, about 10 to about 600 nucleotides in length, about 10 to about 500 nucleotides in length about 10 to about 450 nucleotides in length, about 10 to about 400 nucleotides in length, about 10 to about 350 nucleotides in length, about 10 to about 300 nucleotides in length, about 10 to about 250 nucleotides in length, about 10 to about 200 nucleotides in length, about 10 to about 150 nucleotides in length, about 10 to about 100 nucleotides in length, about 10 to about 90 nucleotides in length, about 10 to about 80 nucleotides in length, about 10 to about 70 nucleotides in length, about 10 to about 60 nucleotides in length, about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, about 18 to about 28 nucleotides in length, about 15 to about 26 nucleotides in length, and all oligonucleotides intermediate in length of the sizes specifically disclosed to the extent that the oligonucleotide is able to achieve the desired result. In further embodiments, an oligonucleotide of the disclosure is about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 40 nucleotides in length, about 5 to about 30 nucleotides in length, about 5 to about 20 nucleotides in length, about 5 to about 10 nucleotides in length, and all oligonucleotides intermediate in length of the sizes specifically disclosed to the extent that the oligonucleotide is able to achieve the desired result. Accordingly, in various embodiments, an oligonucleotide of the disclosure is or is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 300, 400, 500, 600, 700, 800, 900, 1000, or more nucleotides in length. In further embodiments, an oligonucleotide of the disclosure is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or more nucleotides in length. In various embodiments, the shell of oligonucleotides attached to the exterior of the nanoparticle core of the SNA comprises a plurality of oligonucleotides that all have the same length/sequence, while in some embodiments, the plurality of oligonucleotides comprises one or more oligonucleotide that have a different length and/or sequence relative to at least one other oligonucleotide in the plurality. For example and without limitation, in some embodiments the shell of oligonucleotides comprises a plurality of inhibitory oligonucleotides, wherein one inhibitory oligonucleotide has a sequence that is different than at least one other inhibitory oligonucleotide in the plurality.

In some embodiments, one or more oligonucleotides in the shell of oligonucleotides comprises or consists of a (GGX)ₙ nucleotide sequence, wherein n is 2-20 and X is a nucleobase (A, C, T, G, or U). In some embodiments, the (GGX)ₙ nucleotide sequence is on the 5′ end of the one or more oligonucleotides. In some embodiments, the (GGX)ₙ nucleotide sequence is on the 3′ end of the one or more oligonucleotides. In some embodiments, one or more oligonucleotides in the shell of oligonucleotides comprises or consists of a (GGT)ₙ nucleotide sequence, wherein n is 2-20. In some embodiments, the (GGT)ₙ nucleotide sequence is on the 5′ end of the one or more oligonucleotides. In some embodiments, the (GGT)ₙ nucleotide sequence is on the 3′ end of the one or more oligonucleotides.

In some embodiments, an oligonucleotide in the shell of oligonucleotides is a targeting oligonucleotide, such as an aptamer. In some embodiments, the aptamer is a forced intercalation (FIT) aptamer as described herein below. Accordingly, all features and aspects of oligonucleotides described herein (e.g., length, type (DNA, RNA, modified forms thereof), optional presence of spacer) also apply to aptamers. Aptamers are oligonucleotide sequences that can be evolved to bind to various target analytes of interest. Aptamers may be single stranded, double stranded, or partially double stranded.

Spacers. In some aspects and embodiments, one or more oligonucleotides in the shell of oligonucleotides that is attached to the nanoparticle core of a SNA comprise a spacer. “Spacer” as used herein means a moiety that serves to increase distance between the nanoparticle core and the oligonucleotide, or to increase distance between individual oligonucleotides when attached to the nanoparticle core in multiple copies, or to improve the synthesis of the SNA. Thus, spacers are contemplated being located between an oligonucleotide and the nanoparticle core.

In some aspects, the spacer when present is an organic moiety. In some aspects, the spacer is a polymer, including but not limited to a water-soluble polymer, a nucleic acid, a polypeptide, an oligosaccharide, a carbohydrate, a lipid, an ethylglycol, or a combination thereof. In any of the aspects or embodiments of the disclosure, the spacer is an oligo (ethylene glycol)-based spacer. In various embodiments, an oligonucleotide comprises 1, 2, 3, 4, 5, or more spacer (e.g., Spacer-18 (hexaethyleneglycol)) moieties. In further embodiments, the spacer is an alkane-based spacer (e.g., C12). In some embodiments, the spacer is an oligonucleotide spacer (e.g., T5). An oligonucleotide spacer may have any sequence that does not interfere with the ability of the oligonucleotides to become bound to the nanoparticle core or to a target. In certain aspects, the bases of the oligonucleotide spacer are all adenylic acids, all thymidylic acids, all cytidylic acids, all guanylic acids, all uridylic acids, or all some other modified base.

In various embodiments, the length of the spacer is or is equivalent to at least about 2 nucleotides, at least about 3 nucleotides, at least about 4 nucleotides, at least about 5 nucleotides, 5-10 nucleotides, 10 nucleotides, 10-30 nucleotides, or even greater than 30 nucleotides.

SNA surface density. Generally, a surface density of oligonucleotides that is at least about 0.5 pmol/cm² will be adequate to provide a stable SNA. In further embodiments, a surface density of oligonucleotides that is at least about 1 pmol/cm², 1.5 pmol/cm², or 2 pmoles/cm² will be adequate to provide a stable SNA. In some aspects, the surface density of a SNA of the disclosure is at least 15 pmoles/cm². Methods are also provided wherein the oligonucleotide is attached to the nanoparticle core of the SNA at a surface density of about 0.5 pmol/cm² to about 1000 pmol/cm², or about 2 pmol/cm² to about 200 pmol/cm², or about 10 pmol/cm² to about 100 pmol/cm². In some embodiments, the surface density is about 1.7 pmol/cm². In some embodiments, the surface density is about 2 pmol/cm². In further embodiments, the surface density is at least about 0.5 pmol/cm², at least about 0.6 pmol/cm², at least about 0.7 pmol/cm², at least about 0.8 pmol/cm², at least about 0.9 pmol/cm², at least about 1 pmol/mc², at least about 1.5 pmol/cm², at least about 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more. In further embodiments, the surface density is less than about 2 pmol/cm², less than about 3 pmol/cm², less than about 4 pmol/cm², less than about 5 pmol/cm², less than about 6 pmol/cm², less than about 7 pmol/cm², less than about 8 pmol/cm², less than about 9 pmol/cm², less than about 10 pmol/cm², less than about 15 pmol/cm², less than about 19 pmol/cm², less than about 20 pmol/cm², less than about 25 pmol/cm², less than about 30 pmol/cm², less than about 35 pmol/cm², less than about 40 pmol/cm², less than about 45 pmol/cm², less than about 50 pmol/cm², less than about 55 pmol/cm², less than about 60 pmol/cm², less than about 65 pmol/cm², less than about 70 pmol/cm², less than about 75 pmol/cm², less than about t 80 pmol/cm², less than about 85 pmol/cm², less than about 90 pmol/cm², less than about 95 pmol/cm², less than about 100 pmol/cm², less than about 125 pmol/cm², less than about 150 pmol/cm², less than about 175 pmol/cm², less than about 200 pmol/cm², less than about 250 pmol/cm², less than about 300 pmol/cm², less than about 350 pmol/cm², less than about 400 pmol/cm², less than about 450 pmol/cm², less than about 500 pmol/cm², less than about 550 pmol/cm², less than about 600 pmol/cm², less than about 650 pmol/cm², less than about 700 pmol/cm², less than about 750 pmol/cm², less than about 800 pmol/cm², less than about 850 pmol/cm², less than about 900 pmol/cm², less than about 950 pmol/cm², or less than about 1000 pmol/cm².

Alternatively, the density of oligonucleotide attached to the SNA is measured by the number of oligonucleotides attached to the SNA. With respect to the surface density of oligonucleotides attached to a SNA of the disclosure, it is contemplated that a SNA as described herein comprises or consists of about 1 to about 5,000, about 1 to about 2,500, or about 1 to about 500 oligonucleotides on its surface. In various embodiments, a SNA comprises about 10 to about 5000, or about 10 to about 4000, or about 10 to about 3000, or about 10 to about 2000, or about 10 to about 1000, or about 10 to about 500, or about 10 to about 300, or about 10 to about 200, or about 10 to about 190, or about 10 to about 180, or about 10 to about 170, or about 10 to about 160, or about 10 to about 150, or about 10 to about 140, or about 10 to about 130, or about 10 to about 120, or about 10 to about 110, or about 10 to about 100, or 10 to about 90, or about 10 to about 80, or about 10 to about 70, or about 10 to about 60, or about 10 to about 50, or about 10 to about 40, or about 10 to about 30, or about 10 to about 20, or about 75 to about 200, or about 75 to about 150, or about 100 to about 200, or about 150 to about 200 oligonucleotides in the shell of oligonucleotides attached to the nanoparticle core. In some embodiments, a SNA comprises about 80 to about 140 oligonucleotides in the shell of oligonucleotides attached to the nanoparticle core. In further embodiments, a SNA comprises at least about 5, 10, 20, 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 500, 1000, 2000, 3000,4000, or 5000 oligonucleotides in the shell of oligonucleotides attached to the nanoparticle core. In further embodiments, a SNA consists of 5, 10, 20, 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 500, 1000, 2000, 3000, 4000, or 5000 oligonucleotides in the shell of oligonucleotides attached to the nanoparticle core. In still further embodiments, the shell of oligonucleotides attached to the nanoparticle core of the SNA comprises 2, 3, 4, 5, 6, 7, 8, 9, 10, 11, 12, 13, 14, 15, 16, 17, 18, 19, 20, 50, 60, 70, 75, 80, 90, 100, 150, 160, 170, 175, 180, 190, 200, 500, 1000, 2000, 3000, 4000, 5000, or more oligonucleotides. In some embodiments, the shell of oligonucleotides attached to the nanoparticle core of the SNA consists of 2, 3, 4, 5, 6, 7, 8, 9, 10, 11, 12, 13, 14, 15, 16, 17, 18, 19, 20, 30, 40, 50, 60, 70, 75, 80, 90, 100, 150, 160, 170, 175, 180, 190, 200, 500, 1000, 2000, 3000, 4000, or 5000 oligonucleotides. In some embodiments, the shell of oligonucleotides comprises about 10 to about 80 oligonucleotides. In some embodiments, the shell of oligonucleotides comprises or consists of about 75 oligonucleotides. In some embodiments, a SNA comprising a liposomal or lipid nanoparticle core (which may, in various embodiments, be about or less than about 150 nanometers in diameter or about or less than about 100 nanometers in diameter or about or less than about 80 nanometers in diameter or about or less than about 70 nanometers in diameter) comprises about 10 to about 2,000 oligonucleotides, or about 10 to about 1,000 oligonucleotides, or about 10 to about 100 oligonucleotides, or about 10 to about 80 oligonucleotides, or about 10 to about 40 oligonucleotides on its surface. In some embodiments, a SNA comprising a metallic (e.g., gold) core comprises about 70 to about 120 oligonucleotides on its surface. In some embodiments, a SNA comprising a polymer (e.g., PLGA) core comprises about 10 to about 5000 oligonucleotides, or about 10 to about 4000, or about 10 to about 3000, or about 10 to about 2000, or about 10 to about 1000, or about 10 to about 900, or about 10 to about 800, or about 10 to about 700, or about 10 to about 600, or about 10 to about 500, or about 10 to about 400 oligonucleotides, or about 10 to about 300 oligonucleotides, or about 10 to about 200 oligonucleotides, or about 10 to about 100 oligonucleotides on its surface.

### Uses of SNAs in Gene Regulation

In some aspects of the disclosure, the shell of oligonucleotides that is attached to the external surface of the nanoparticle core comprises one or more inhibitory oligonucleotides designed to inhibit target gene expression. In some embodiments, each oligonucleotide in the shell of oligonucleotides attached to the external surface of a SNA of the disclosure is an inhibitory oligonucleotide. Regular SNAs (not salted with CaCl₂)) can accumulate in endosomes (such as late endosomes) and only a small fraction are able to escape into the cytosol of a cell. As described herein (see, e.g., Example 3), however, accumulation of SNAs of the disclosure (CaCl₂) salted SNAs) in endosomes is decreased relative to accumulation of regular SNAs. In various embodiments, accumulation of a SNA of the disclosure within an endosome is reduced by at least 5%, at least 10%, at least 15%, at least 20%, at least 25%, at least 30%, or more compared to accumulation of regular (e.g., NaCl-salted) SNAs within the endosome. Such decreased accumulation in endosomes advantageously results in enhanced cytosolic delivery of SNAs of the disclosure and enhanced gene silencing activity compared to regular (e.g., NaCl-salted) SNAs. Such decreased accumulation in endosomes is also advantageously extended to molecular probes to improve cytosolic mRNA detection.

Accordingly, in some aspects the disclosure provides a method of inhibiting expression of a gene product comprising the step of hybridizing a target polynucleotide encoding the gene product with a SNA of the disclosure, wherein hybridizing between the target polynucleotide and one or more oligonucleotides in the shell of oligonucleotides occurs over a length of the target polynucleotide with a degree of complementarity sufficient to inhibit expression of the gene product. In some embodiments, the hybridizing occurs intracellularly. In some aspects, the disclosure provides a method of inhibiting expression of a gene product comprising the step of contacting a cell comprising the gene product with a spherical nucleic acid (SNA) of the disclosure, thereby inhibiting expression of the gene product. In some embodiments, expression of the gene product is inhibited in vitro. In some embodiments, expression of the gene product is inhibited in vivo.

Methods for inhibiting gene product expression provided herein include those wherein expression of the target gene product is inhibited by about or at least about 5%, about or at least about 10%, about or at least about 15%, about or at least about 20%, about or at least about 25%, about or at least about 30%, about or at least about 35%, about or at least about 40%, about or at least about 45%, about or at least about 50%, about or at least about 55%, about or at least about 60%, about or at least about 65%, about or at least about 70%, about or at least about 75%, about or at least about 80%, about or at least about 85%, about or at least about 90%, about or at least about 95%, about or at least about 96%, about or at least about 97%, about or at least about 98%, about or at least about 99%, or 100% compared to gene product expression in the absence of a SNA of the disclosure. In other words, methods provided embrace those which results in essentially any degree of inhibition of expression of a target gene product. In some embodiments, expression of the gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to inhibition of gene product expression of a NaCl-salted spherical nucleic acid (SNA) under identical conditions.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of SNA and a specific oligonucleotide.

In various aspects, the methods include use of an inhibitory oligonucleotide which is 100% complementary to the target polynucleotide, i.e., a perfect match, while in other aspects, the oligonucleotide is at least (meaning greater than or equal to) about 95% complementary to the polynucleotide over the length of the oligonucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to the polynucleotide over the length of the oligonucleotide to the extent that the oligonucleotide is able to achieve the desired degree of inhibition of a target gene product.

The percent complementarity is determined over the length of the oligonucleotide. For example, given an antisense compound in which 18 of 20 nucleotides of the inhibitory oligonucleotide are complementary to a 20 nucleotide region in a target polynucleotide of 100 nucleotides total length, the oligonucleotide would be 90 percent complementary. In this example, the remaining noncomplementary nucleotides may be clustered or interspersed with complementary nucleobases and need not be contiguous to each other or to complementary nucleotides. Percent complementarity of an inhibitory oligonucleotide with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

The oligonucleotide utilized in such methods is either RNA or DNA. The RNA can be an inhibitory oligonucleotide, such as an inhibitory RNA (RNAi) that performs a regulatory function, and in various embodiments is a small inhibitory RNA (siRNA), a single-stranded RNA (ssRNA), a ribozyme, or a combination thereof. Alternatively, the RNA is microRNA that performs a regulatory function. The DNA is, in some embodiments, an antisense DNA. In some embodiments, the RNA is a piwi-interacting RNA (piRNA). In various embodiments, the inhibitory oligonucleotide is an antisense oligonucleotide, small interfering RNA (siRNA), an aptamer, a short hairpin RNA (shRNA), a DNAzyme, or an aptazyme.

### Therapeutic Agents

In any of the aspects or embodiments of the disclosure, a therapeutic agent is administered separately from a SNA of the disclosure. Thus, in various embodiments, a therapeutic agent is administered before, after, or concurrently with a SNA of the disclosure to treat a disorder (e.g., cancer).

In some aspects, the SNAs provided herein optionally further comprise a therapeutic agent, or a plurality thereof. The therapeutic agent is, in various embodiments, simply associated with an oligonucleotide in the shell of oligonucleotides attached to the exterior of the nanoparticle core of the SNA, and/or the therapeutic agent is associated with the nanoparticle core of the SNA, and/or the therapeutic agent is encapsulated in the nanoparticle core of the SNA. In some embodiments, the therapeutic agent is associated with the end of an oligonucleotide in the shell of oligonucleotides that is not attached to the nanoparticle core (e.g., if the oligonucleotide is attached to the nanoparticle core through its 3′ end, then the therapeutic agent is associated with the 5′ end of the oligonucleotide). Alternatively, in some embodiments, the therapeutic agent is associated with the end of an oligonucleotide in the shell of oligonucleotides that is attached to the nanoparticle core (e.g., if the oligonucleotide is attached to the nanoparticle core through its 3′ end, then the therapeutic agent is associated with the 3′ end of the oligonucleotide). In some embodiments, the therapeutic agent is covalently associated with an oligonucleotide in the shell of oligonucleotides that is attached to the exterior of the nanoparticle core of the SNA. In some embodiments, the therapeutic agent is non-covalently associated with an oligonucleotide in the shell of oligonucleotides that is attached to the exterior of the nanoparticle core of the SNA. However, it is understood that the disclosure provides SNAs wherein one or more therapeutic agents are both covalently and non-covalently associated with oligonucleotides in the shell of oligonucleotides that is attached to the exterior of the nanoparticle core of the SNA. It will also be understood that non-covalent associations include hybridization, protein binding, and/or hydrophobic interactions.

Therapeutic agents contemplated by the disclosure include without limitation a protein (e.g., a therapeutic protein), a growth factor, a hormone, an interferon, an interleukin, an antibody or antibody fragment, a small molecule, a peptide, an antibiotic, an antifungal, an antiviral, a chemotherapeutic agent, or a combination thereof. In some embodiments, the therapeutic agent is an anti-programmed cell death protein 1 (PD-1) antibody.

The term “small molecule,” as used herein, refers to a chemical compound or a drug, or any other low molecular weight organic compound, either natural or synthetic. By “low molecular weight” is meant compounds having a molecular weight of less than 1500 Daltons, typically between 100 and 700 Daltons.

### Uses of SNAs in Immune Regulation

Toll-like receptors (TLRs) are a class of proteins, expressed in sentinel cells, that play a key role in regulation of innate immune system. The mammalian immune system uses two general strategies to combat infectious diseases. Pathogen exposure rapidly triggers an innate immune response that is characterized by the production of immunostimulatory cytokines, chemokines and polyreactive IgM antibodies. The innate immune system is activated by exposure to Pathogen Associated Molecular Patterns (PAMPs) that are expressed by a diverse group of infectious microorganisms. The recognition of PAMPs is mediated by members of the Toll-like family of receptors. TLR receptors, such as TLR 8 and TLR 9 that respond to specific oligonucleotides are located inside special intracellular compartments, called endosomes. The mechanism of modulation of, for example and without limitation, TLR 8 and TLR 9 receptors, is based on DNA-protein interactions.

As described herein, synthetic immunostimulatory oligonucleotides that contain CpG motifs that are similar to those found in bacterial DNA stimulate a similar response of the TLR receptors. Thus, CpG oligonucleotides of the disclosure have the ability to function as TLR agonists. Other TLR agonists contemplated by the disclosure include, without limitation, single-stranded RNA and small molecules (e.g., R848 (Resiquimod)). Therefore, immunomodulatory (e.g., immunostimulatory) oligonucleotides have various potential therapeutic uses, including treatment of diseases (e.g., cancer).

Accordingly, in some embodiments, the disclosure provides methods of utilizing SNAs comprising one or more immunostimulatory oligonucleotides as described herein. In some embodiments, the methods up-regulate the Toll-like-receptor activity through the use of a TLR agonist, and comprise contacting a cell having a toll-like receptor with a SNA of the disclosure, thereby modulating the activity and/or the expression of the toll-like receptor. In various embodiments, the toll-like receptor is toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, toll-like receptor 13, or a combination thereof.

In some aspects, the disclosure provides a method for up-regulating activity of a toll-like receptor (TLR), comprising contacting a cell having the toll-like receptor with a CaCl₂) salted SNA or composition of the disclosure, thereby up-regulating activity of the TLR. In some embodiments, the shell of oligonucleotides comprises one or more oligonucleotides that is a TLR agonist. In further embodiments, the toll-like receptor is toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, toll-like receptor 13, or a combination thereof. In some embodiments, the method is performed in vitro. In some embodiments, the method is performed in vivo.

In further aspects, the disclosure provides a method for down-regulating activity of a toll-like receptor (TLR), comprising contacting a cell having the toll-like receptor with a CaCl₂) salted SNA or composition of the disclosure, thereby down-regulating activity of the TLR. In some embodiments, the shell of oligonucleotides comprises one or more oligonucleotides that is a TLR antagonist. In various embodiments, the toll-like receptor is toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, toll-like receptor 13, or a combination thereof. In some embodiments, the method is performed in vitro. In some embodiments, the method is performed in vivo.

### Uses of SNAs to Treat a Disorder

In some aspects, a SNA of the disclosure is used to treat a disorder. Thus, in some aspects, the disclosure provides methods of treating a disorder comprising administering an effective amount of a SNA of the disclosure to a subject (e.g., a human subject) in need thereof, wherein the administering treats the disorder. In various embodiments, the disorder is cancer, an infectious disease, an autoimmune disease, or a combination thereof. In some aspects, the disclosure provides methods of treating a cancer comprising administering to a subject (e.g., a human subject) an effective amount of a SNA of the disclosure, thereby treating the cancer in the subject. In various embodiments, the cancer is bladder cancer, breast cancer, cervical cancer, colon cancer, rectal cancer, endometrial cancer, glioblastoma, kidney cancer, leukemia, liver cancer, lung cancer, melanoma, lymphoma, non-Hodgkin lymphoma, osteocarcinoma, ovarian cancer, pancreatic cancer, prostate cancer, thyroid cancer, and human papilloma virus-induced cancer, or a combination thereof.

### Uses of SNAs in Detection

In some aspects, the SNAs of the disclosure comprise one or more targeting oligonucleotide and are useful in detecting a target analyte. In some embodiments, the SNAs of the disclosure are useful in nanoflare technology. The nanoflare has been previously described in the context of polynucleotide-functionalized nanoparticles for fluorescent detection of target molecule levels inside a living cell (described in U.S. Patent Application Publication No. 20100129808, incorporated herein by reference in its entirety). In this system the “flare” is detectably labeled and is, in some embodiments, one strand of a double-stranded oligonucleotide (or a portion of a single-stranded oligonucleotide) that is labeled with a detectable marker and is displaced or released from the SNA by an incoming target polynucleotide. In further embodiments, the detectable marker is any fluorescent marker known in the art (e.g., cyanine, fluorescein). It is thus contemplated that the nanoflare technology is useful in the context of the SNAs described herein.

In various aspects, SNAs of the disclosure are also useful in forced intercalation (FIT) aptamer technology and FIT flare technology. FIT aptamers are described in International Publication No. WO 2020/257674, which is incorporated by reference herein in its entirety. In various embodiments, FIT aptamer technology comprises methods of detecting the presence of a target analyte by contacting the target analyte with an aptamer comprising a detectable marker situated at an internal location within the aptamer, wherein the contacting results in binding of the target analyte to the aptamer, thereby producing a detectable change in the marker (through, e.g., restriction of internal rotation of the marker). In further aspects, SNAs of the disclosure are useful in FIT flare technology, which is described in International Publication No. WO 2021/177996, incorporated by reference herein in its entirety. In various embodiments, FIT flare technology comprises methods for detecting a target analyte by contacting the target analyte with a spherical nucleic acid (SNA), wherein the SNA comprises a nanoparticle core and an oligonucleotide attached to the nanoparticle core, wherein the oligonucleotide comprises a detectable marker situated at an internal location within the oligonucleotide and the contacting results in binding of the target analyte to the oligonucleotide, resulting in a detectable change (e.g., an increase in fluorescence) and thereby detecting the target analyte. In various embodiments, the detectable marker is thiazole orange (TO), quinoline blue, quinoline violet, thiazole red, a derivative thereof, or a cyanine derivative.

Target analytes contemplated by the disclosure include without limitation a protein, an ion, a small molecule, a lipid, a carbohydrate, an oligosaccharide, a cell, an oligonucleotide, or a combination thereof.

Thus, in some aspects the disclosure provides a method for detecting a target analyte comprising the step of contacting the target analyte with a CaCl₂) salted SNA or composition of the disclosure, wherein one or more oligonucleotides in the shell of oligonucleotides comprises a detectable marker, wherein the contacting results in binding of the target analyte to the one or more oligonucleotides in the shell of oligonucleotides comprising the detectable marker, resulting in a detectable change and thereby detecting the target analyte. Such methods are useful, for example and without limitation, to determine the intracellular concentration of a target analyte. In some embodiments, each oligonucleotide in the shell of oligonucleotides comprises a detectable marker. In some embodiments, the detectable marker is attached to a polynucleotide hybridized to the one or more oligonucleotides in the shell of oligonucleotides, wherein association of the one or more oligonucleotides in the shell of oligonucleotides with the target analyte releases the polynucleotide hybridized to the one or more oligonucleotides in the shell of oligonucleotides and the detectable marker is detectable after release. In some embodiments, each oligonucleotide in the shell of oligonucleotides has a polynucleotide hybridized thereto, wherein the polynucleotide comprises a detectable marker, wherein association of one or more or all oligonucleotides in the shell of oligonucleotides with the target analyte releases the polynucleotide hybridized to the oligonucleotides in the shell of oligonucleotides and the detectable marker is detectable after release. In further embodiments, the detectable marker attached to the polynucleotide is quenched when the polynucleotide with the detectable marker is hybridized to the one or more oligonucleotides in the shell of oligonucleotides. In still further embodiments, the detectable marker is detectable only when the one or more oligonucleotides in the shell of oligonucleotides is associated with the target analyte. In some embodiments, the detectable marker is quenched when the one or more oligonucleotides in the shell of oligonucleotides is not associated with the target analyte. In various embodiments, the detectable marker is situated at an internal location within the oligonucleotide. In some embodiments, the binding results in restriction of internal rotation of the detectable marker. In further embodiments, the detectable marker is thiazole orange (TO), quinoline blue, quinoline violet, thiazole red, a derivative thereof, or a cyanine derivative. In some embodiments, the detectable change is proportional to concentration of the target analyte. In various embodiments, the target analyte is a protein, an ion, a small molecule, a lipid, a carbohydrate, an oligosaccharide, a cell, an oligonucleotide, or a combination thereof. In further embodiments, the target analyte is RNA. In some embodiments, the target analyte is mRNA. In some embodiments, the target analyte is cytosolic mRNA. In various embodiments, the methods are in vitro methods. In some embodiments, the methods are in vivo methods.

### EXAMPLES

### Example 1

In this Example, it is demonstrated that siRNA functionalized poly-lactic-co-glycolic acid (PLGA) spherical nucleic acids (SNAs) salted with calcium chloride (CaCl₂)) induced enhanced gene regulation activity of SNAs without causing any significant cellular toxicity.

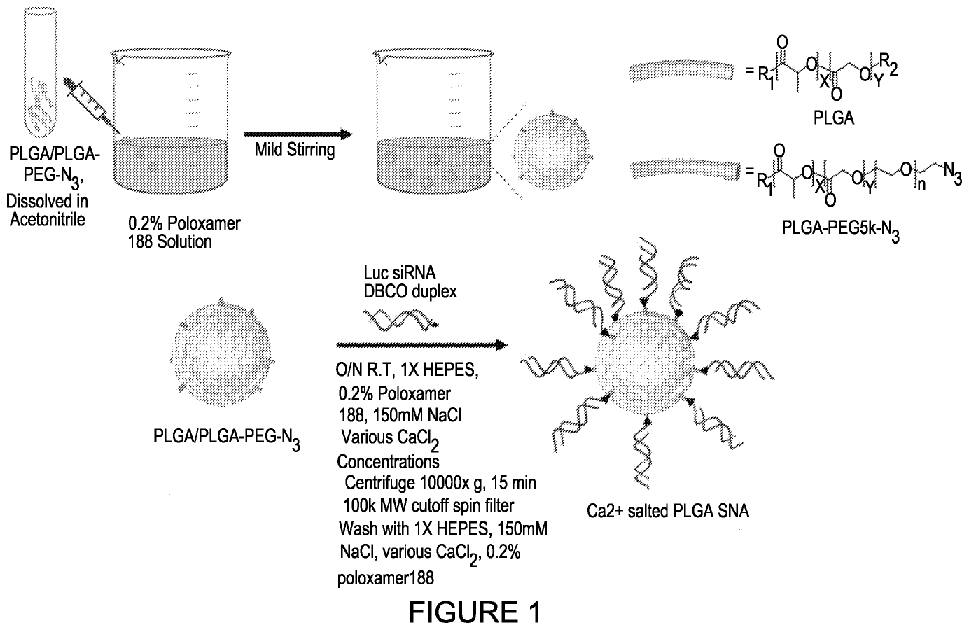

siRNA functionalized PLGA SNAs were synthesized via the salt-aging method using calcium chloride (CaCl₂)). siRNA duplex, a surfactant, sodium chloride (NaCl) and calcium chloride (CaCl₂)), and PLGA nanoparticle core were combined and incubated with shaking overnight. FIG. 1 depicts how a PLGA/PLGA-PEG-N₃ nanoparticle (NP) core was synthesized using a nanoprecipitation method. SNAs then were washed with HEPES-buffer saline (HBS) containing CaCl₂) using a centrifugal filter. Specifically, 9.75 mg of carboxylic acid (COOH) terminated PLGA (Sigma Aldrich) and 5.25 mg of PLGA-PEG5000-N₃ (Akina) was co-dissolved in acetonitrile (ACN) (6 mL), which was then drop wise injected into 0.3% (v/v) Poloxamer 188 solution (24 mL) in a 100 ml glass beaker under stirring (900 rpm) with a magnetic stirrer. The resulting solution was allowed to evaporate for 2 hours in a fume hood. The NP solution was then concentrated to 1 ml in nanopure water using a 15 mL 100 kDa cut off Amicon filter. After determining the nanoparticle concentration by Malvern NanoSight NS300, the concentration of surface azide (N₃) on the PLGA core was calculated based on previously reported method [Luk, B. T., Hu, C. M. J., Fang, R. H., Dehaini, D., Carpenter, C., Gao, W., & Zhang, L. (2014). Interfacial interactions between natural RBC membranes and synthetic polymeric nanoparticles. Nanoscale, 6 (5), 2730-2737]. Then CaCl₂) salted PLGA SNA was formed utilizing copper-free click chemistry by adding dibenzocyclooctyne (DBCO) modified siRNA duplex so that the resulting solution contained 0.1M pH 7.4 HEPES buffered saline (HBS) with 0.3% (v/v) Poloxamer 188, 350 mM NaCl and different concentrations of CaCl₂). The reaction mixture was incubated for 24 hours under room temperature. After 24 hours of incubation, the un-reacted oligonucleotides were removed by centrifugation for 15 min at 10000×g using 500 μL 100 kDa cutoff spin filter (Amicon) four times using 1×HBS with 0.3% (v/v) Poloxamer 188 with different concentrations of CaCl₂).

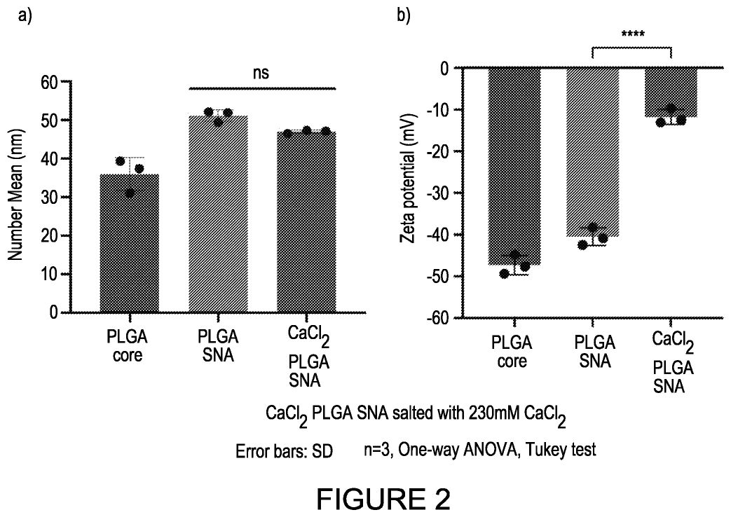

Particle size distribution and the surface charge (zeta potential) of the PLGA/PLGA-PEG-N₃ nanoparticle (NP) core was assessed. PLGA SNAs that were salted without CaCl₂) and CaCl₂) salted PLGA SNAs were measured using Malvern Zetasizer Ultra Red. All samples were diluted 10-fold in 100 μL water solution and the hydrodynamic diameter (HD) measurements were derived from the number average value measured at 25° C. The reported DLS size for each sample was based on at least five measurements per run in triplicates. The surface charge (zeta potential) of the particles was measured in triplicates using the DTS 1070 zeta cell, each run was measured in water solution (800 μL, dilution factor 80) at 25° C., and 10 to 50 measurements were taken using the automated settings in the ZS Xplorer software provided with the Malvern Zetasizer Ultra Red. CaCl₂) salted PLGA SNAs were monodispersed and showed similar size compared to regular PLGA SNAs (without CaCl₂)) (FIG. 2a). However, CaCl₂) salted PLGA SNAs showed an increase in zeta potential, indicating adsorption of Ca²⁺ ions on the phosphate backbone of the oligonucleotide shell (FIG. 2b).

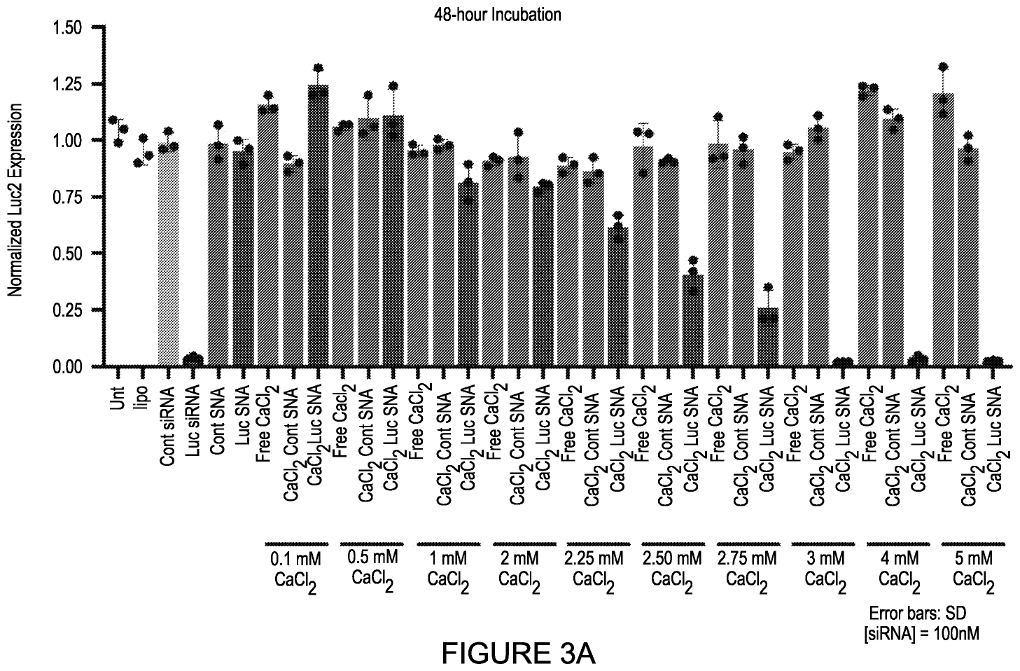

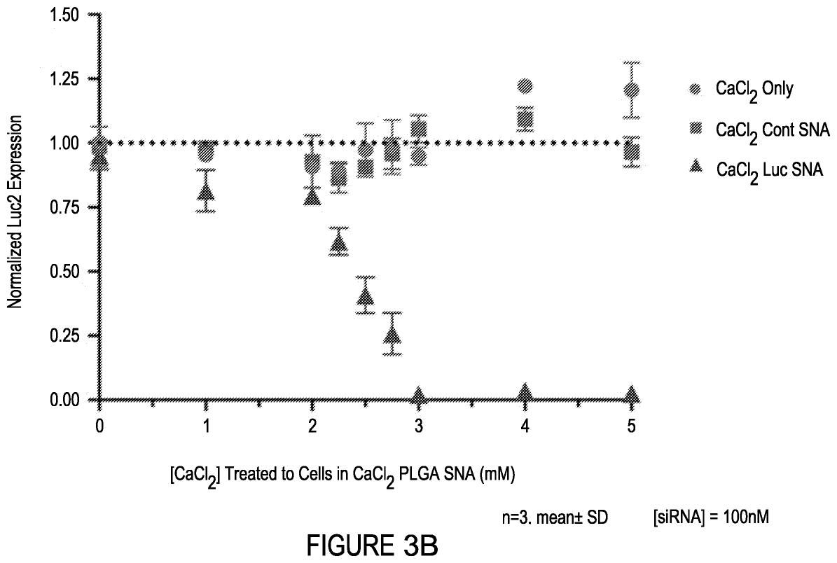

To test CaCl₂) salted PLGA SNA's functionality, in vitro protein knockdown experiment was performed by treating the SNAs in full serum media for 48 hours using a U87 (glioblastoma)-Luciferase reporter cell line to down regulate the expression of the luciferase. Thus, CaCl₂) dose dependent activity of the CaCl₂) salted PLGA SNA was determined using luciferase reporter assay. U87-MG-Luc2 glioblastoma luciferase reporter cell line (ATCC) was seeded in a 96-well plate at a density of 12,000 cells per well with a total volume of 200 μL using MEM cell culture media supplemented with 10% fetal bovine serum, 8 μg/mL blasticidin and 1% penicillin-streptomycin. After overnight incubation, PLGA SNAs salted in different concentrations of CaCl₂) (see Table 1) were treated to the cells where siRNA concentration treated to the cells were kept constant at 100 nM. To treat the cells at a siRNA concentration of 100 nM in a 200 μL 96 well plate, a certain volume of the CaCl₂) PLGA SNA was treated to the cells. By way of example, 2.61 μL of 230 mM CaCl₂) salted PLGA SNA was required to treat the cells so that the final concentration of siRNA is 100 nM in a 200 μL total volume in a 96 well. As Molarity (concentration) is mole/volume, the concentration of CaCl₂) treated to the cells would be (230 mM*2.61 μL)/200 μL=approximately 3 mM. After 48 hours of incubation, the wells were washed with HBS twice, and 90 μL of fresh cell culture media indicated above was added to the wells. Subsequently, 10 μL of PrestoBlue™ Cell viability reagent (Thermo Fisher) was added to measure the relative cell viability compared to the untreated wells. After a 30-minute incubation at 37° C., fluorescence intensity was measured at excitation/emission wavelengths of 560 nm/590 nm using Cytation 5 Multimode Plate Reader (Biotek). After measuring the cell viability, wells were then washed with 150 μL of HBS three times. 100 μL of cell culture media was added to the wells and then 100 μL of Bright-Glo™ Luciferase Assay solution (Promega) was added to measure the luminescence of the cells. Luc2 protein expression was analyzed in arbitrary units where luminescence value was normalized to fluorescence value from PrestoBlue assay. Then the extent of Luc2 protein knockdown was determined by normalizing the arbitrary units to that of the untreated control group. The results were plotted in bar graph as well as in XY axis plot graph. The experiment was performed in triplicates and the error was calculated as standard deviation of the mean. CaCl₂) salted PLGA SNAs that targeted luciferase caused 95% knockdown efficiency compared to the untreated cells and whereas control (non-targeting) CaCl₂) salted PLGA SNAs did not cause any knockdown, confirming CaCl₂) salted PLGA SNAs' sequence specific knockdown activity (FIG. 3A and FIG. 3B). Moreover, CaCl₂) salted PLGA SNA knockdown efficiency was significantly improved compared to regular PLGA SNAs by 20-fold.

TABLE 1
Concentrations of CaCl₂ that were tested.

| | |
|---|---|
| | Concentration of CaCl₂ that |
| Final CaCl₂ in each well | PLGA SNA is salted in |

| | | | |
|---|---|---|---|
| 0.1 | mM | 7 | mM |
| 0.5 | mM | 36 | mM |
| 1 | mM | 70 | mM |
| 2 | mM | 130 | mM |
| 2.25 | mM | 160 | mM |
| 2.5 | mM | 184 | mM |
| 2.75 | mM | 210 | mM |
| 3 | mM | 230 | mM |
| 4 | mM | 290 | mM |
| 5 | mM | 333 | mM |

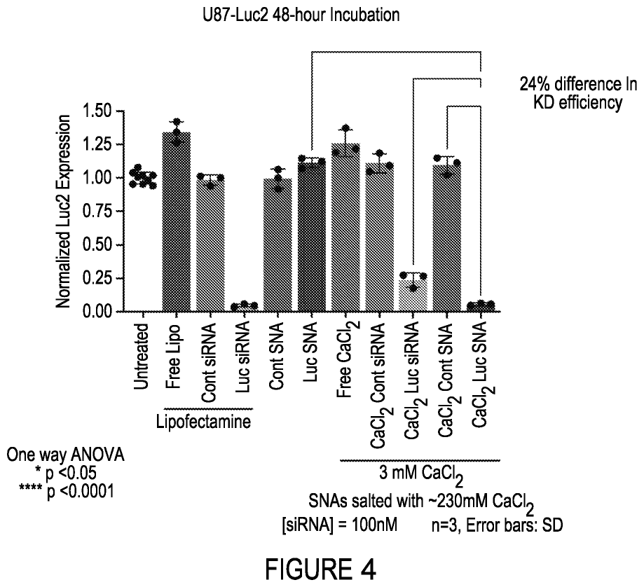

After determining that PLGA SNA salted with 230 mM CaCl₂) gave maximum down regulation of Luc2 protein, a luciferase reporter assay was once again conducted to compare the gene regulation activity of CaCl₂) salted PLGA SNAs and CaCl₂) salted linear siRNA duplex. The experiment was performed in triplicates and the error was calculated as standard deviation of the mean. As shown in FIG. 4, CaCl₂) salted PLGA SNAs did not induce any significant cellular toxicity.

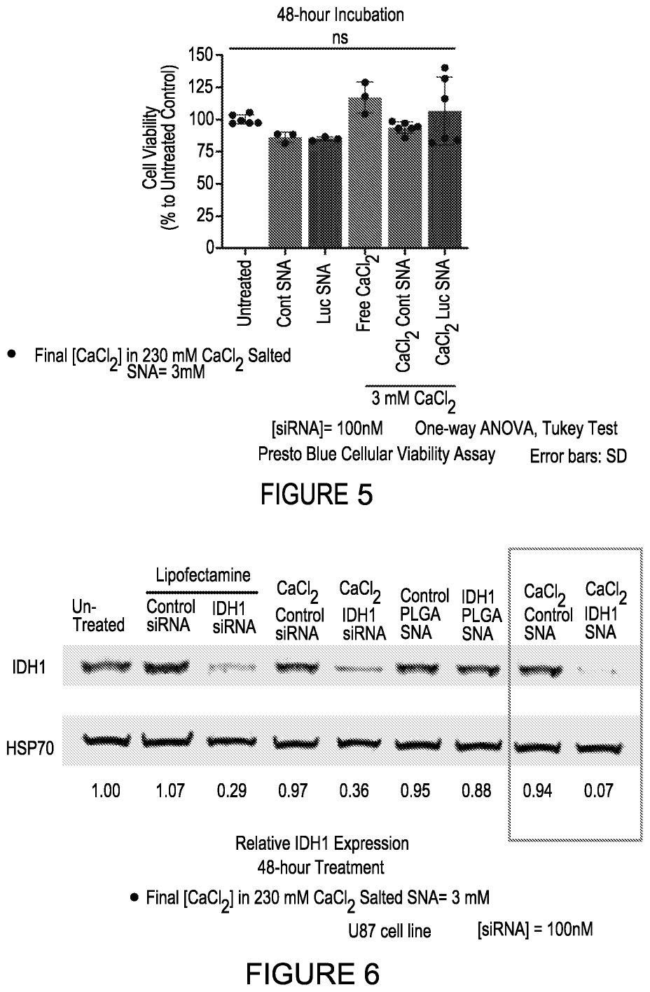

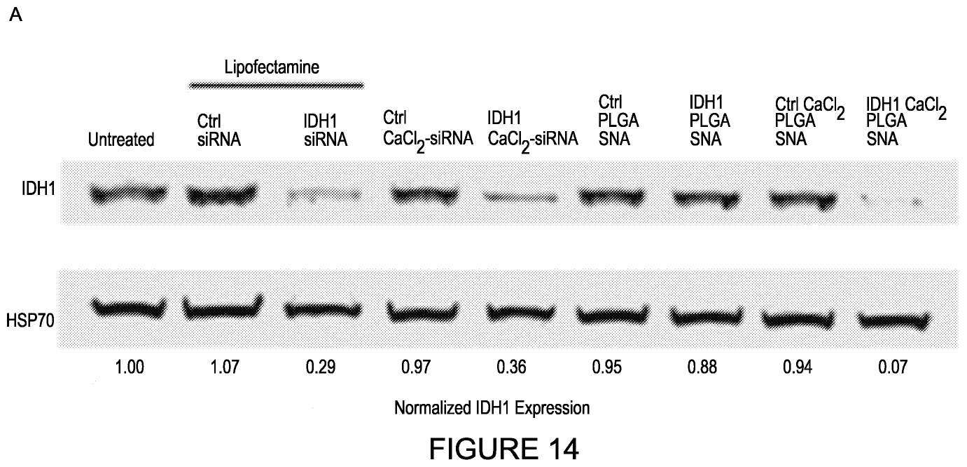

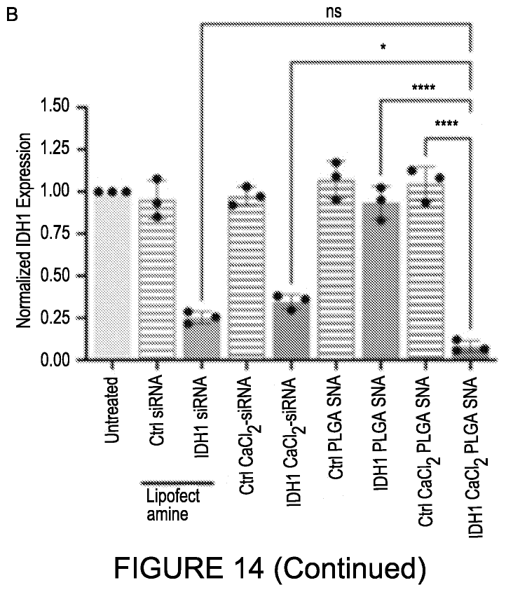

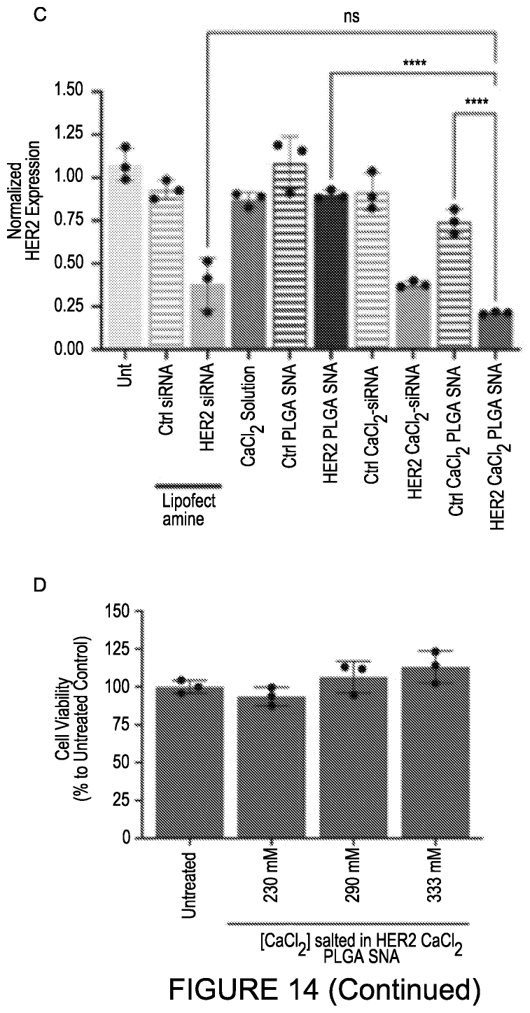

CaCl₂) salted PLGA SNAs enhanced gene regulation functionality was further validated by down regulating IDH1 protein expression in U87 glioblastoma cell line (FIG. 5) and down regulating HER2 expression in SKOV-3 ovarian cancer cell line (FIG. 6). See also FIG. 14.

The cell viability of U87-MG-Luc2 cells was determined using PrestoBlue™ cell viability reagent (Thermo Fisher). Cells were seeded in a 96-well plate at a density of 12,000 cells per well. After overnight incubation, cells were treated under treatment conditions as indicated in FIG. 5. After 48 hours of incubation, cells were washed with HBS twice, and 90 μL of fresh cell culture media was added to the wells. Subsequently, 10 μL of PrestoBlue™ Cell viability reagent (Thermo Fisher) was added to measure the relative cell viability compared to the untreated wells. After a 30-minute incubation at 37° C., fluorescence intensity was measured at excitation/emission wavelengths of 560 nm/590 nm using BioTek Synergy Microplate Reader. Cell viability was normalized to the untreated control group and plotted as a percentage of cell viability. The experiment was performed in triplicates and the error was calculated as standard deviation of the mean.

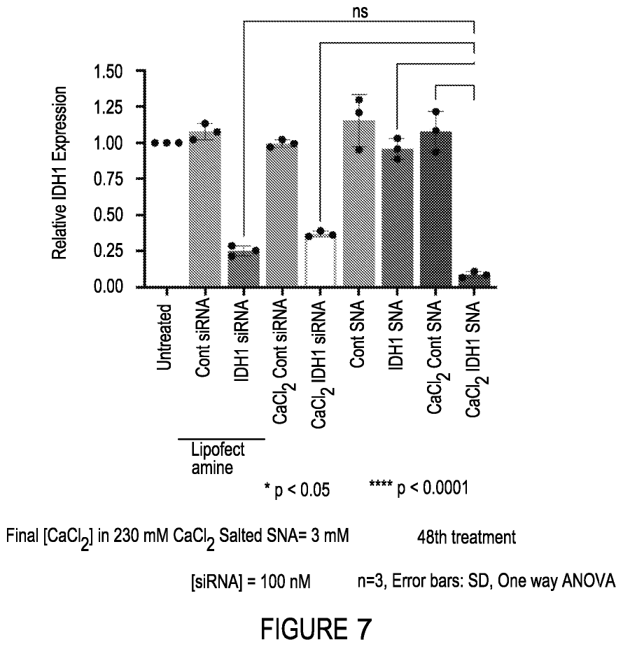

To determine protein knockdown of IDH1 (isocitrate dehydrogenase 1) using CaCl₂) salted PLGA SNAs, 100,000 U87-MG (ATCC) cells were seeded in a 12-well plate with a total volume of 2 mL using MEM cell culture media supplemented with 10% fetal bovine serum and 1% penicillin-streptomycin and were incubated overnight. Cells were then treated with CaCl₂) salted PLGA SNAs and other control groups and were incubated for 48-hours. After washing the wells with 1×HBS twice, protein lysates were then extracted using RIPA buffer that includes protease inhibitor. After measuring the protein lysate concentration by BCA assay, 30 μg of protein lysate per treatment sample was separated using gel electrophoresis. Then the gel was transferred to nitrocellulose membrane using iBlot® 2 Gel Transfer Device (Life Technologies). Then the membrane was blocked with Intercept® (TBS) blocking buffer (LI-COR) in room temperature for 1 h with shaking and was incubated overnight at 4° C. with shaking using the following antibodies: rabbit anti-IDH1 (Cell Signaling Technology, 1:1000 dilution in blocking buffer, total 10 mL) and mouse IgG1 anti-HSP70 (BD biosciences, 1:2000 dilution in blocking buffer, total 10 mL). After the blots were washed with 0.1% Tween-20 in 1× phosphate buffered saline (1×PBST) three times for 5 min, the membranes were incubated with IRDye® 800CW-conjugated goat anti-rabbit secondary antibody (LI-COR, 1:2000 dilution in blocking buffer, 10 mL) and IRDye® 800CW-conjugated goat anti-mouse IgG1 secondary antibody (LI-COR, 1:2000 dilution in blocking buffer, 10 mL) for 1 hr in room temperature protected from light, with shaking. Then the nitrocellulose membrane washed with 1×PBST three times for 5 min. To remove residual Tween-20, the membrane was rinsed in deionized water three times before scanning. Then the blot image was acquired using Odyssey® CLx Imager (Li—COR) at 169 μm resolution in the 800 nm fluorescence channel. Then the band intensity of the blot was quantified by Image J (NIH) normalized to the untreated control group. Western blot was performed (FIG. 6) in triplicates and the results were plotted in a bar graph (FIG. 7) and the error was calculated as standard deviation of the mean.

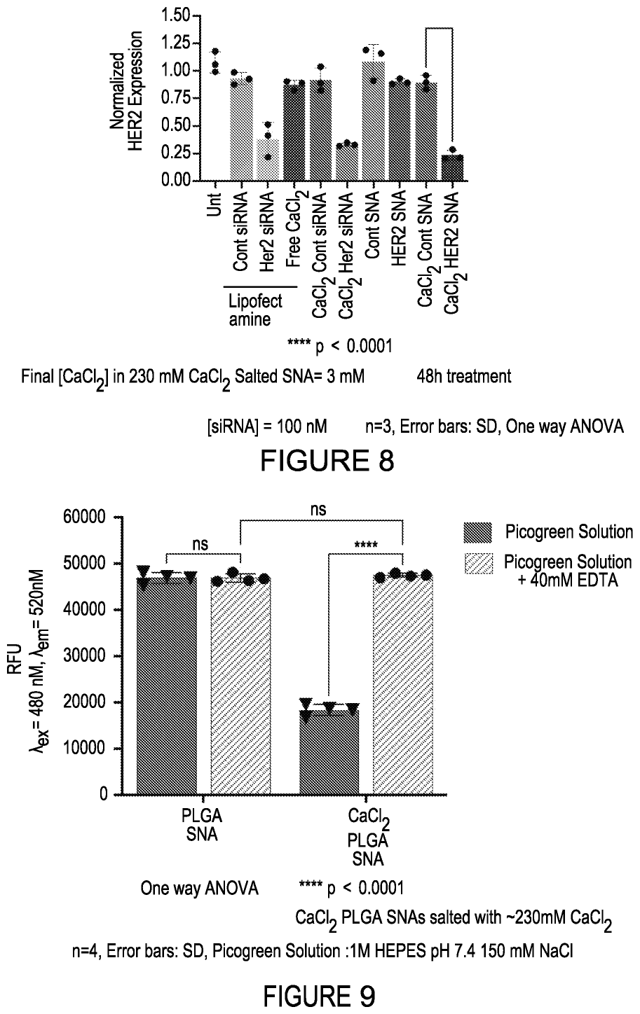

To determine protein knockdown of HER2 (human epidermal growth factor 2) using CaCl₂) salted PLGA SNAs, 12,000 SKOV-3 ovarian cancer cells (ATCC) were seeded in a 96-well plate in a total volume of 200 μL using DMEM cell culture media supplemented with 10% fetal bovine serum and 1% penicillin-streptomycin and were incubated overnight. Cells where then treated with CaCl₂) salted PLGA SNAs and other treatment groups for 48 hours. The wells were then washed with HBS twice followed by two washes with 0.05% Tween-20 in 1× phosphate buffered saline (PBS) and then in 1×PBS once and the plate was incubated with Intercept® (TBS) blocking buffer (LI-COR) in room temperature for 90 minutes with shaking. The cells were then incubated with HER2 antibody (Cell signaling technology, 1:200 dilution in blocking buffer, 200 μL) for 2 hours in room temperature with shaking. Then the wells were washed with 1×PBST three times and was incubated with a 200 μL blocking buffer solution containing 2 μg/mL of IRDye 800CW goat anti-rabbit secondary antibody (LI-COR) and 1 nM CellTag 700 (LI-COR) for 1 hour protected from light, with shaking. Then the wells were washed with 1×PBST 3 times and imaged on an Odyssey CLx system (LI-COR). HER2 protein expression was calculated in arbitrary units by normalizing fluorescence at 800 nm (HER2) to fluorescence at 700 nm (cell viability number). Then the extent of HER2 protein knockdown was determined by normalizing HER2 protein expression to that of the untreated control group and the results were plotted in a bar graph (FIG. 8).

### Example 2

To verify the association of Ca²⁺ ions in the CaCl₂) salted PLGA SNA, a PicoGreen exclusion assay was conducted, modifying a previously reported ethidium bromide exclusion assay. Electrostatic adsorption of the Ca²⁺ ions to the phosphate backbone of the siRNA duplexes would physically screen the oligonucleotide shell, preventing the PicoGreen reagent from intercalating within the siRNA duplex and consequently would lead to a decrease in PicoGreen fluorescence intensity. However, in the presence of ethylenediaminetetraacetic acid (EDTA) in the PicoGreen solution, EDTA would chelate the Ca²⁺ ions enabling the Picogreen reagent to intercalate within the siRNA duplex and lead to an increase in fluorescence intensity. FIG. 9 demonstrates that in CaCl₂) salted PLGA SNAs, fluorescence intensity substantially decreased to about 60% compared to the fluorescence intensity of the PLGA SNAs. When the CaCl₂) salted PLGA SNAs were treated with Picogreen solution that contains 40 mM EDTA, the fluorescence intensity significantly increased to a similar level to that of the PLGA SNA, suggesting association of the Ca²⁺ ions with the oligonucleotide shell of the SNA architecture.

Methods: Picogreen Exclusion Assay to Evaluate Association of Ca²⁺ ion within the oligonucleotide shell in the CaCl₂) Salted PLGA SNA

The complexation of Ca²⁺ to the PLGA SNA siRNA was determined by Quant-iT™ PicoGreen™ (Invitrogen) exclusion assay. 50 μL of CaCl₂) salted PLGA SNA and PLGA SNA was first added to the 96 well plate and then 150 μL of PicoGreen™ solution containing either 1×HBS or 1×HBS+40 mm EDTA was added to the 96 well plate. The plate was then read at the excitation/emission wavelengths of 480 nm/520 nm using a Biotek synergy plate reader.

### Example 3

This Example provides further detail and experimental data related to Example 1 and Example 2.

The therapeutic potential of small interfering RNAs (siRNAs) is limited by their poor stability, low cellular uptake, and poor endosomal escape. When formulated as spherical nucleic acids (SNAs), siRNAs are resistant to nuclease degradation, enter cells without the need for transfection agents (e.g., cationic polymers or lipids), and exhibit enhanced activity compared to their linear counterparts; however, the gene silencing activity of SNAs can be limited by endosomal entrapment, a problem that impacts many nanoparticle-based constructs used for gene regulation. In an effort to increase cytosolic delivery, SNAs were prepared using 230 mM CaCl₂) instead of the conventionally used 500 mM NaCl. The divalent Ca²⁺ ions have a higher affinity for the siRNA functionalized to the multivalent SNA and stabilize the negatively charged nucleic acid shell. Like their NaCl counterparts, these CaCl₂)-salted constructs exhibit 2.5-fold enhanced cellular uptake compared to CaCl₂)-salted linear siRNA in U87-MG human glioblastoma cells. Moreover, at early timepoints, the cellular entry of the CaCl₂) salted structures exceed that of the NaCl-salted entities by 36-fold. Importantly, confocal microscopy studies show 22% decrease in accumulation of CaCl₂)-salted SNAs within the late endosomes compared to NaCl salted SNAs, indicating increased cytosolic delivery. Consistent with this conclusion, CaCl₂)-salted SNAs comprised of siRNA and antisense DNA all exhibit enhanced gene silencing activity, as compared with SNAs synthesized with NaCl regardless of sequence or cell line (U87-MG and SK-OV-3) studied. In addition, this method has been extended to molecular probes, such as forced intercalation (FIT) flares (see, e.g., U.S. Patent Application Publication No. 2023/0088835, incorporated by reference herein in its entirety), to improve cytosolic mRNA detection.

Because SNAs are multivalent and are already capable of readily entering over 60 cell lines, including human derived cell lines,⁷ it was hypothesized that they would have a higher affinity for Ca²⁺ and that Ca²⁺-complexed SNAs would have the ability to co-deliver Ca²⁺ and gene-regulatory oligonucleotides into cells through endocytosis. Such properties could potentially lead to improved cytosolic delivery and corresponding gene regulation activity. Herein, the cellular uptake and gene regulation activities of poly-(lactic-co-glycolic) acid (PLGA) SNAs salted with CaCl₂) to take advantage of PLGA's biocompatibility and biodegradability was investigated.⁶⁴⁻⁶⁶ In a U87-MG human glioblastoma cell line, these constructs exhibited enhanced cellular uptake at early time points (36-fold), reduced accumulation (22%) within the late endosomes compared to NaCl-salted PLGA SNAs, and markedly improved gene silencing capabilities (8 to 18-fold enhancement). Moreover, no apparent toxicity was observed across three different cell lines involving three different targets. Collectively, these data established the CaCl₂) salting of SNAs as a method for improving cytosolic delivery of SNAs for multiple applications, regardless of nucleic acid sequence. Moreover, it is shown herein that this method can be extended to molecular probes such as forced intercalation (FIT) flares⁶⁷ to improve cytosolic mRNA detection.

### Results and Discussion

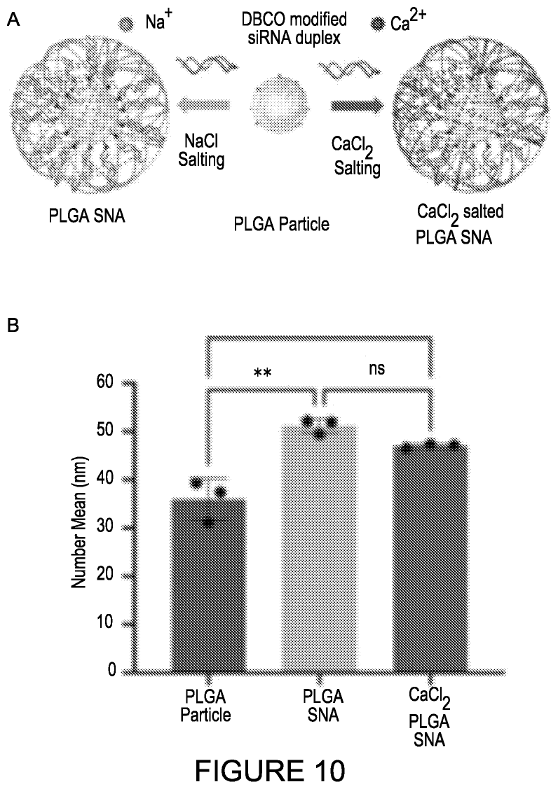

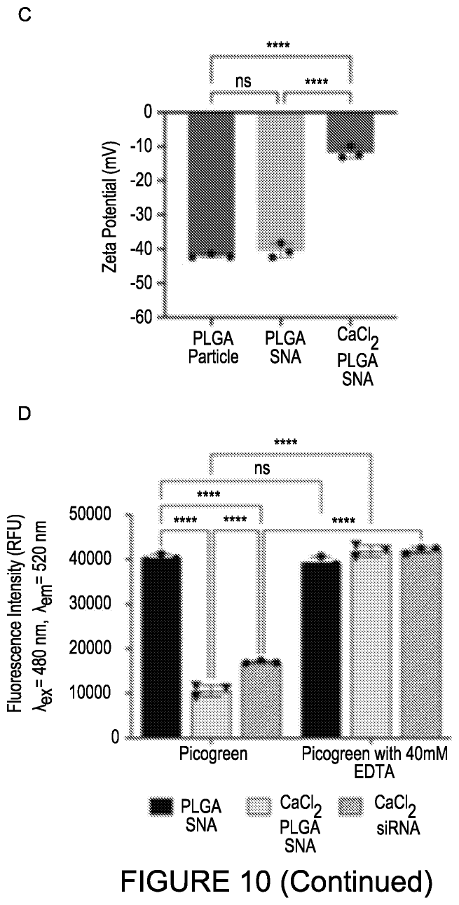

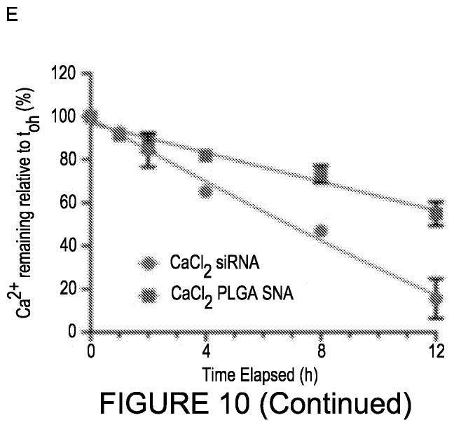

Synthesis and characterization of CaCl₂)-salted PLGA SNAs. To prepare CaCl₂)-salted PLGA SNAs, spherical PLGA particles (36.0±4.3 nm) that incorporate PLGA-b-poly(ethylene glycol)-azides were synthesized using a nanoprecipitation method and subsequently functionalized with dibenzocyclooctyne (DBCO)-modified siRNA duplexes via copper-free click chemistry.⁶⁶ In order to generate the CaCl₂)-salted PLGA SNAs (termed CaCl₂) PLGA SNAs), instead of the conventionally used 500 mM NaCl,^{8,40,66} CaCl₂) solution was used to raise the total CaCl₂) concentration to 230 mM (FIG. 10A).

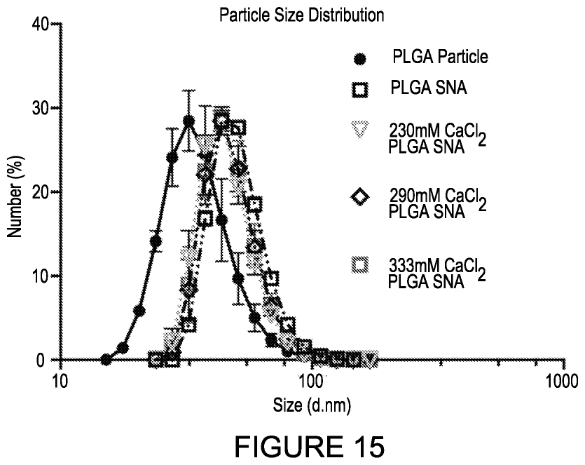

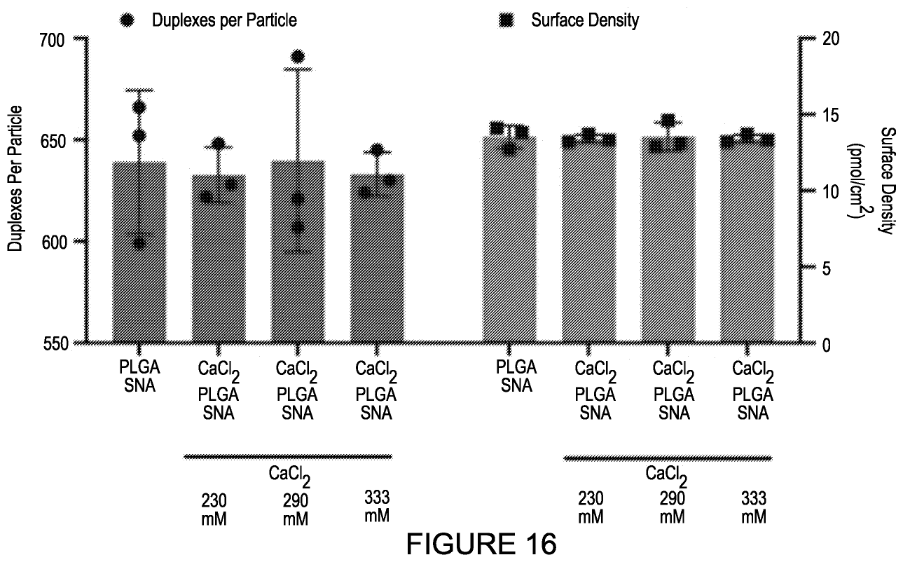

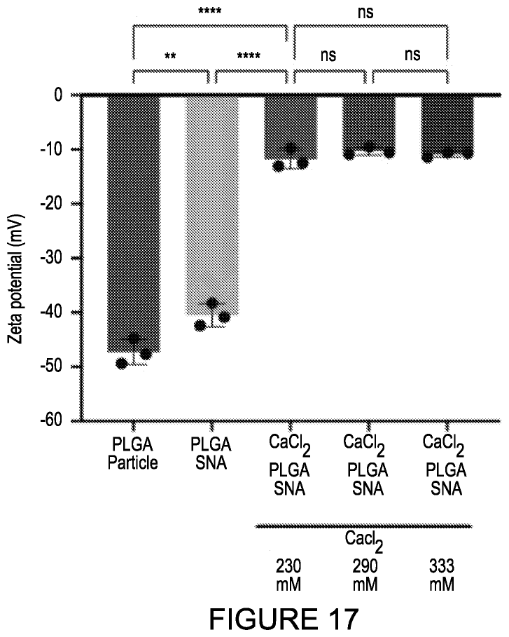

To characterize these SNAs, dynamic light scattering (DLS) was first used to analyze the size of the PLGA nanoparticles before and after functionalization with siRNAs. DLS measurements showed that the hydrodynamic diameter of the standard NaCl-salted PLGA SNAs (henceforth termed PLGA SNAs) (51.2±1.5 nm, PDI: 0.045±0.010) was on average 16 nm larger than that of the bare PLGA particles, which is expected based on the calculated length of the 23 base pair siRNA duplexes and assuming one RNA base pair length is 0.34 nm (23×0.34×2=15.64 nm, FIG. 10B⁶⁸. The hydrodynamic diameters and polydispersity indices (PDIs) of the PLGA SNAs and CaCl₂) PLGA SNAs were not significantly different (CaCl₂)-salted PLGA SNAs: 47.0±0.4 nm, PDI: 0.078±0.009; FIG. 15, Table 3. Furthermore, the siRNA duplex loading and surface density of the PLGA SNAs and the CaCl₂) PLGA SNAs were not significantly different; with average loading of 639.1±35.3 and 632.7±13.4 duplexes per particle, with a surface density of 13.5±0.7 and 13.6±0.3 pmol/cm² for PLGA SNAs and the CaCl₂) PLGA SNAs, respectively (FIG. 16). The zeta potential of the azide-modified PLGA particles was −42.1±0.6 mV due to the negatively charged terminal carboxylic groups of PLGA chains on the nanoparticle surface as reported previously.^{69,70} The zeta potential of the PLGA SNAs was −40.5±2.1 mV; with no significant change in zeta potential compared to PLGA particles (FIG. 10C). CaCl₂) PLGA SNAS exhibited a significantly less negative zeta potential (−11.8±1.8 mV), similar to the trend observed for the previously reported CaCl₂)-siRNAs (−11.9±0.9 mV);⁶² which is a consequence of Ca²⁺ association with RNA shell (FIG. 10C, FIG. 17, Table 3).

TABLE 3
Size, poly dispersity index (PDI), and zeta potential
of PLGA particles, PLGA SNAs (NaCl-salted), and CaCl₂ salted
PLGA SNAs (mean ± standard deviation, n = 3).

| | Size | | |
|---|---|---|---|
| | (Diameter, | | Zeta Potential |
| | nm) | PDI | (mV) |
| PLGA core | 36.0 ± 4.3 | 0.147 ± 0.004 | −47.3 ± 2.3 |
| PLGA SNAs | 51.2 ± 1.5 | 0.045 ± 0.010 | −40.5 ± 2.1 |
| 230 mM | 47.0 ± 0.4 | 0.078 ± 0.009 | −11.8 ± 1.8 |
| CaCl₂ PLGA SNAs | | | |
| 290 mM | 47.7 ± 1.8 | 0.064 ± 0.011 | −10.3 ± 0.7 |
| CaCl₂ PLGA SNAs | | | |
| 333 mM | 47.8 ± 2.2 | 0.070 ± 0.026 | −10.9 ± 0.4 |
| CaCl₂ PLGA SNAs | | | |

To further verify the association of Ca²⁺ ions with the SNAs, a modified ethidium bromide (EtBr) exclusion assay^{63,71} was conducted by employing PicoGreen™ in place of EtBr due to its lower limit of detection.^{72,73} The fluorescence intensity of the PicoGreen™ increases when it intercalates into double-stranded nucleic acids.⁷⁴ Therefore, it was hypothesized that the electrostatic adsorption of the divalent Ca²⁺ ions to the phosphate backbone of the siRNA duplexes would compete with and prevent the PicoGreen™ from intercalating within the oligonucleotide shell. Indeed, upon treatment with PicoGreen™, the fluorescence intensity of the CaCl₂) PLGA SNAs was only 25.2±3.1% of that of the PLGA SNAs, whereas the intensity of the CaCl₂)-siRNA (prepared with 100 nM siRNA and 230 mM CaCl₂) equivalent to CaCl₂) PLGA SNAs) was 40.4±0.7% of that of PLGA SNAs (FIG. 10D). However, when the CaCl₂) PLGA SNAs and CaCl₂)-siRNAs were treated with PicoGreen™ solution containing 40 mM ethylenediaminetetraacetic acid (EDTA), which chelates Ca²⁺ ions, the fluorescence intensities of CaCl₂) PLGA SNAs and CaCl₂)—SiRNAs increased to a level similar to that of the PLGA SNAs. These data were consistent with the conclusion that Ca²⁺ ions are indeed associated with the oligonucleotide shell of the SNA and supported the hypothesis that the multivalent structure of the SNA, comprised of densely functionalized oligonucleotide shell, has a higher affinity for Ca²⁺ ions compared to its linear subunits.

To test whether Ca²⁺ ions remained associated with the SNAs upon cellular entry, CaCl₂) PLGA SNAs and CaCl₂)-siRNAs were dialyzed in solutions that mimic the physiological calcium and sodium chloride concentrations (0.1 M HEPES, 137 mM NaCl and 1.8 mM CaCl₂) at 37° C.), and the PicoGreen™ exclusion assay was subsequently performed at various time points up to 12 hours to determine the amount of Ca²⁺ associated with the SNAs or siRNA duplexes at any given time point. Approximately 55.0±5.5% of the Ca²⁺ ions were still bound to the SNAs after 12 hours (FIG. 10E) while 84.4±9.2% was lost from the siRNA at the same time point. This observation indicated that while Ca²⁺ ions dissociated from SNAs under physiological conditions, the rate of dissociation was less than that for siRNA and consistent with the hypothesis that CaCl₂) PLGA SNAs can enter cells with much of the Ca²⁺ still intact. Note that conventional SNAs readily enter cells over the 2-12 hour time window.^{7,8,21,66} This higher affinity for Ca²⁺ ions was attributed to the multivalent three-dimensional SNA structure.

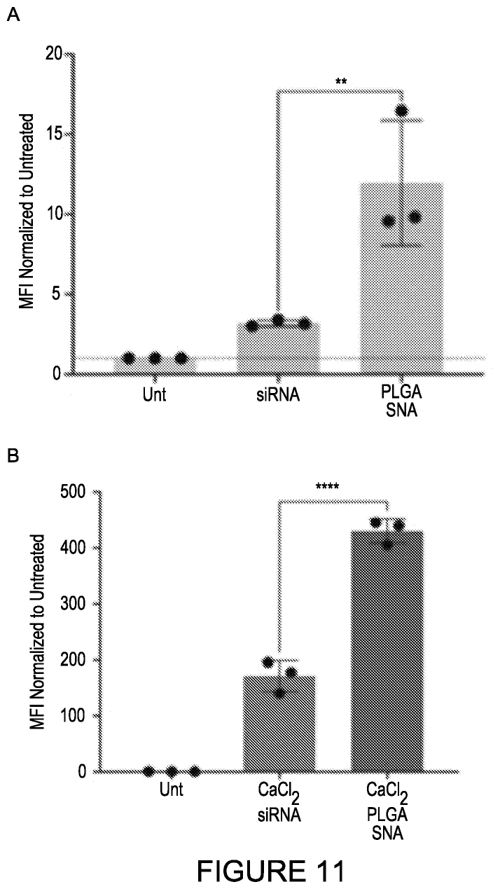

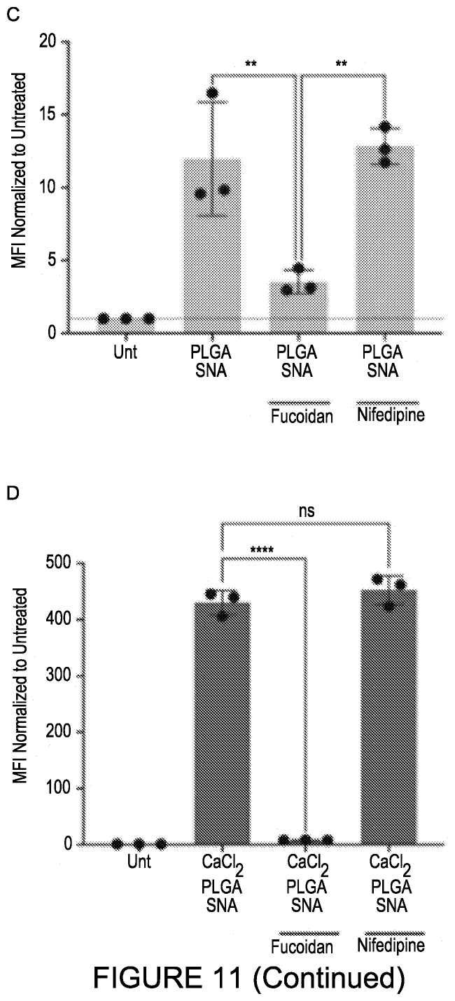

CaCl₂) PLGA SNAs exhibited enhanced cellular uptake compared to PLGA SNAs. The cellular uptake of linear siRNAs or SNAs was analyzed in U87-MG glioblastoma cancer cells using Cyanine 5 (Cy5)-labeled siRNA, and oligonucleotide accumulation was assessed using flow cytometry after 1 hour. The cells treated with PLGA SNAs had 3.7-fold higher median fluorescence intensity (MFI) compared to cells that were treated with linear siRNAs (p<0.01). This observation was consistent with previous reports showing that the multivalent, three-dimensional spherical architecture of the SNAs enhances the cellular uptake of siRNAs (FIG. 11A).^{8,27,66} The CaCl₂) PLGA SNAs had a 36.1-fold higher MFI than PLGA SNAs (p<0.0001) and a 2.5-fold higher MFI than CaCl₂)-siRNAs (p<0.0001) (FIG. 11B). Without being bound by theory, the increased uptake of CaCl₂) PLGA SNAs and CaCl₂)-siRNAs compared to SNAs may be attributed to their more positive zeta potential, a consequence of neutralizing the negative charge of the siRNA with the divalent Ca²⁺ ions, which reduces electrostatic repulsion between the SNAs and the negatively charged cell membrane. Moreover, it has been previously reported that Ca²⁺ ions can also facilitate endocytosis in cells due to the Ca²⁺ concentration gradient between the extracellular and intracellular space.^{75,76} Improved uptake of CaCl₂) PLGA SNAs relative to CaCl₂)-siRNAs can be attributed to the three-dimensional oligonucleotide shell of the SNAs, which facilitates scavenger A mediated endocytosis through multivalent binding.^{8,9}

To probe the mechanism of cellular uptake for the CaCl₂) PLGA SNAs, various pathways were inhibited and the effect of such inhibition on cellular uptake was measured. When pre-treated with fucoidan (FCD), a class A scavenger receptor blocker, PLGA SNA uptake decreased by 70% (p<0.0001) (FIG. 11C), consistent with previous reports that cellular uptake of conventional SNAs is driven by scavenger receptor A-mediated endocytosis.^{8,9} Pre-treatment with FCD also significantly decreased the cellular uptake of CaCl₂) PLGA SNAs by 98% (p<0.0001) (FIG. 11D). Treatment with nifedipine, an L-type Ca²⁺ channel blocker,⁷⁷ did not affect the cellular uptake of CaCl₂) PLGA SNAs, indicating that there is no direct cytosolic uptake of the CaCl₂) PLGA SNAs via Ca²⁺ channels. Moreover, transport via Ca²⁺ channels is size dependent, and SNAs are larger than the Ca²⁺ channel pore size of 5.1-6.2 Å.^{78,79}

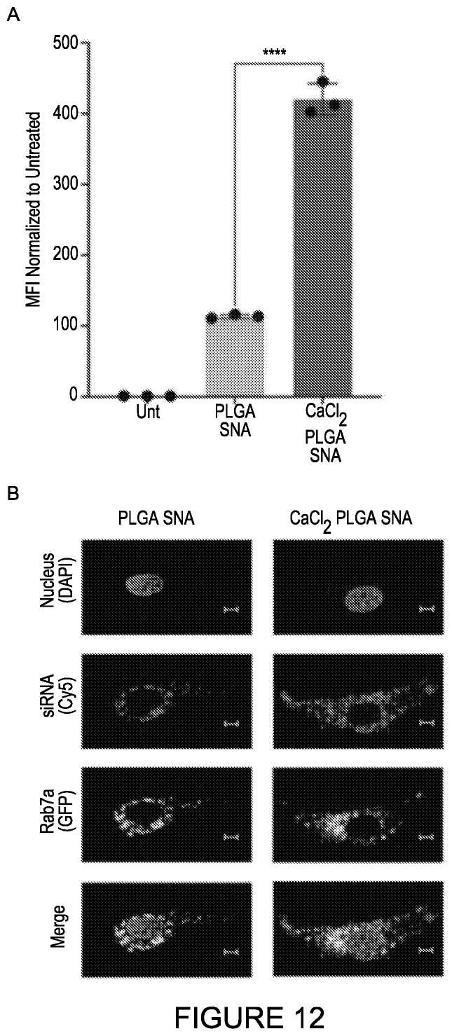

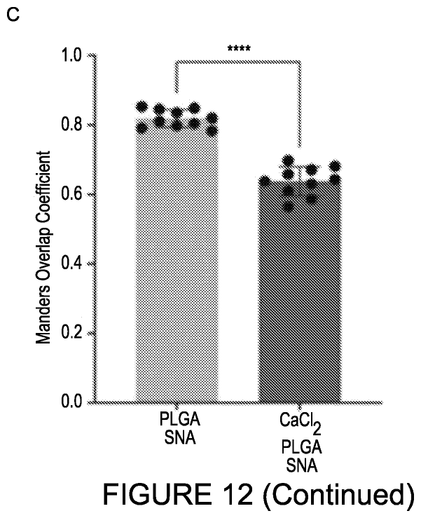

CaCl₂) PLGA SNAs exhibited decreased accumulation in the late endosome compared to SNAs. SNAs have previously been shown to accumulate in late endosomes, and only a small fraction escape to the cytosol to associate with RNAi machinery and facilitate gene regulation.⁴⁰ Thus, it was hypothesized that increased cytosolic delivery would increase the gene regulation activity of SNAs. Hence, to determine whether CaCl₂) PLGA SNAs have improved cytosolic release, the degree of colocalization of PLGA SNAs and CaCl₂) PLGA SNAs was examined with markers of late endosomes via confocal microscopy. The cellular uptake between PLGA SNAs and CaCl₂) PLGA SNAs was first compared after 24 hour treatment via flow cytometry and it was found that cells treated with CaCl₂) PLGA SNAs had 4-fold higher MFI compared to the PLGA SNAs, suggesting that the difference in cellular uptake between PLGA SNAs and CaCl₂) PLGA SNAs is not as significant at 24 hour as compared to the 1 hour time point (FIG. 12A). U87-MG cells were pre-treated with CellLight® Late Endosomes-GFP for 24 hours to express the Rab7a-GFP fusion protein and then treated with PLGA SNAs or CaCl₂) PLGA SNAs containing Cy5-labeled siRNA for an additional 24 hours (FIG. 12B). The cells were then imaged, and quantitative colocalization analysis was conducted by calculating Mander's overlap coefficients (MOC).⁸⁰ CaCl₂) PLGA SNAs showed a significant decrease in siRNA colocalization with the late endosome marker (Rab7a) compared to PLGA SNAs (FIG. 12C), MOC=0.819±0.025 for SNAs and MOC=0.638±0.042 for CaCl₂) PLGA SNAs), suggesting that CaCl₂) PLGA SNAs not only enter cells more readily than PLGA SNAs, but they are also trafficked differently within U87-MG cells, with the escape of SNAs from late endosomes a possible driver of this observation.

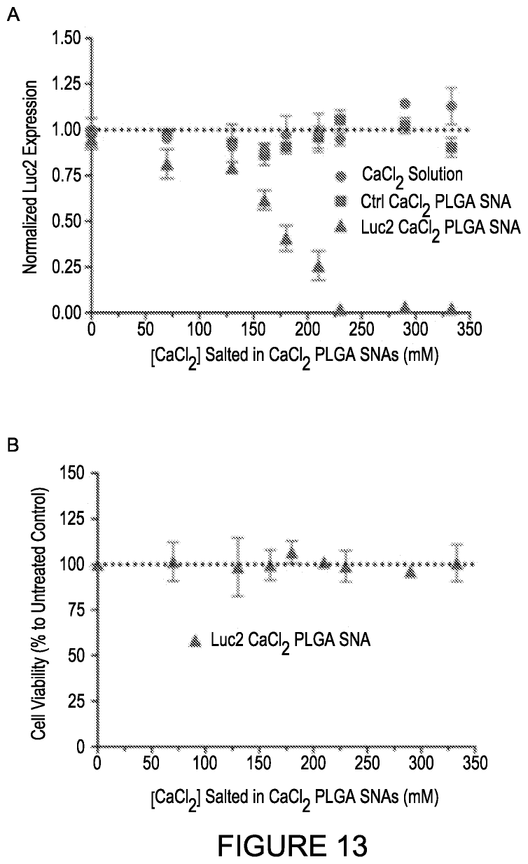

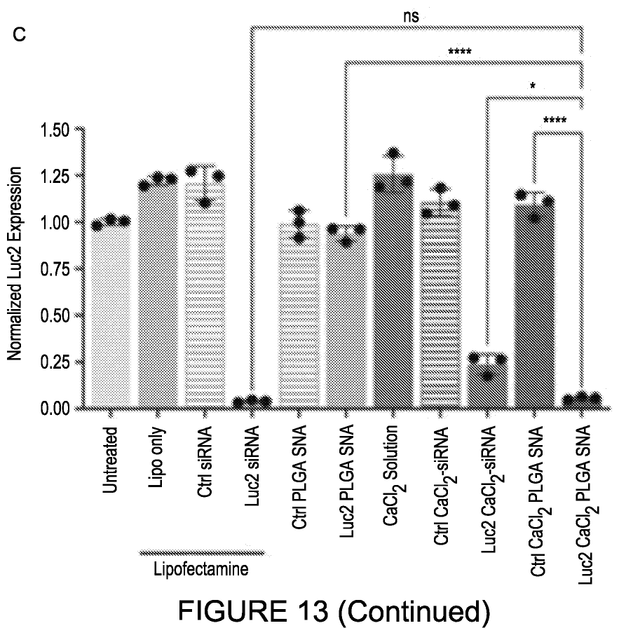

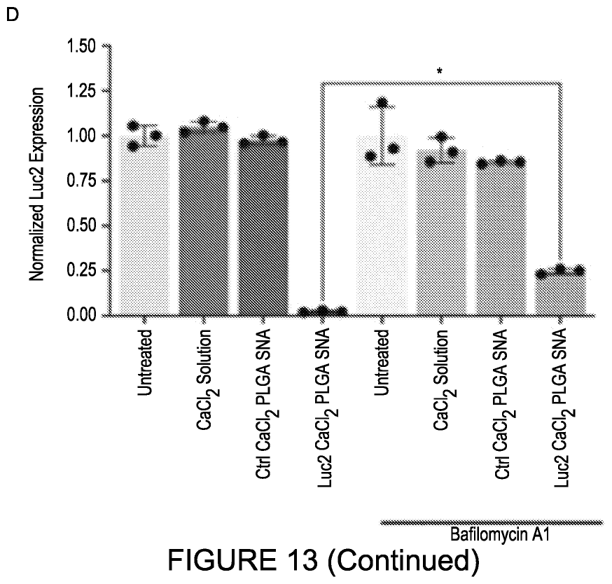

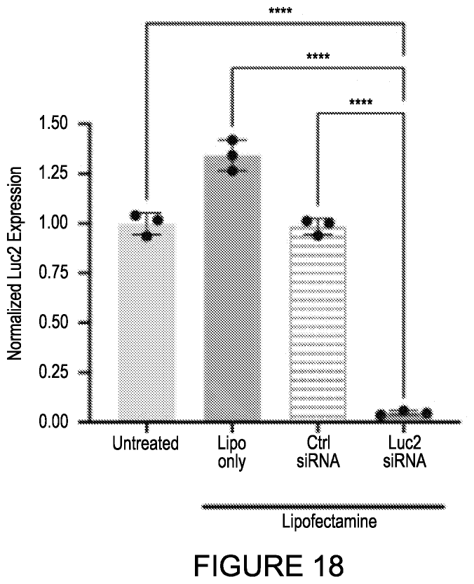

CaCl₂) PLGA SNAs exhibit enhanced silencing of luciferase (Luc2) gene in the U87-MG-Luc2 glioblastoma luciferase reporter cell line. To test the hypothesis that CaCl₂) PLGA SNAs will exhibit improved gene regulation activity compared to conventional PLGA SNAs due to increased cytosolic delivery, the gene silencing activity of the CaCl₂) PLGA SNAs was evaluated using the U87-MG-Luc2 glioblastoma cell line that stably expresses luciferase protein (Luc2) by measuring luciferase luminescence as an indicator of gene expression. It was first confirmed that Luc2-targeting siRNAs led to efficient gene silencing by transfecting the siRNA with Lipofectamine RNAiMAX (a cationic lipid transfection reagent) as compared to cells treated with RNAiMAX only or transfected non-targeting siRNAs (FIG. 18). Next, cells were treated with PLGA SNAs or CaCl₂) PLGA SNAs containing the same Luc2-targeting siRNA (Luc2 PLGA SNA and Luc2 CaCl₂) PLGA SNA) and control non-targeting SNAs (Ctrl PLGA SNA and Ctrl CaCl₂) PLGA SNA) for 48 hours. Cells were treated using a siRNA concentration of 100 nM, while the concentration of CaCl₂) with which the PLGA SNAs were salted was varied. CaCl₂) PLGA SNAs showed CaCl₂) concentration-dependent gene regulation activity, as measured by the luminescence assay (FIG. 13A). The knockdown efficiency of SNAs improved when salted with increasing concentrations of CaCl₂) with approximately 98% knockdown reached at 230 mM CaCl₂). This result supported the conclusion that SNAs with higher amounts of associated Ca²⁺ led to enhanced cytosolic delivery of SNAs. Non-targeting PLGA SNAs and CaCl₂) PLGA SNAs did not show significant Luc2 knockdown activity, confirming that the knockdown is sequence-specific (FIG. 13A) and occurred due to the increased cytosolic delivery of the CaCl₂) PLGA SNAs. Cells treated with CaCl₂) solution at the same concentration used to salt SNAs did not elicit Luc2 gene silencing, indicating that the Luc2 knockdown activity of the CaCl₂) PLGA SNAs is due to RNAi and not CaCl₂) (FIG. 13A).

Because Ca²⁺ ions are an important secondary messenger involved in apoptosis, excessive intracellular Ca²⁺ concentration may cause cell death.⁸¹ PrestoBlue™ cell viability assay was used to determine if the concentration of CaCl₂) added to the cells was cytotoxic. The cells were treated with CaCl₂) PLGA SNAs salted with CaCl₂) concentrations ranging from 0 to 333 mM (FIG. 13B). Consistent with previous findings,⁶³ no significant (p>0.05) cytotoxicity was observed at the concentrations studied. Without being bound by theory, this was likely due to the fact that cells can tolerate elevated Ca²⁺ levels and prevent Ca²⁺-induced apoptosis by removing excess Ca²⁺ via Ca²⁺-ATPase pumps on the plasma membrane and mitochondrial calcium uniporter pumps on the mitochondrial membrane.^{39,82}

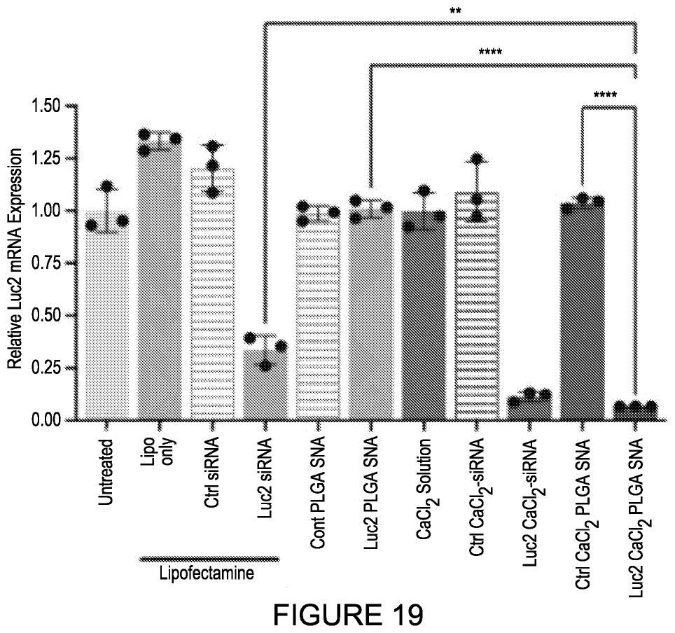

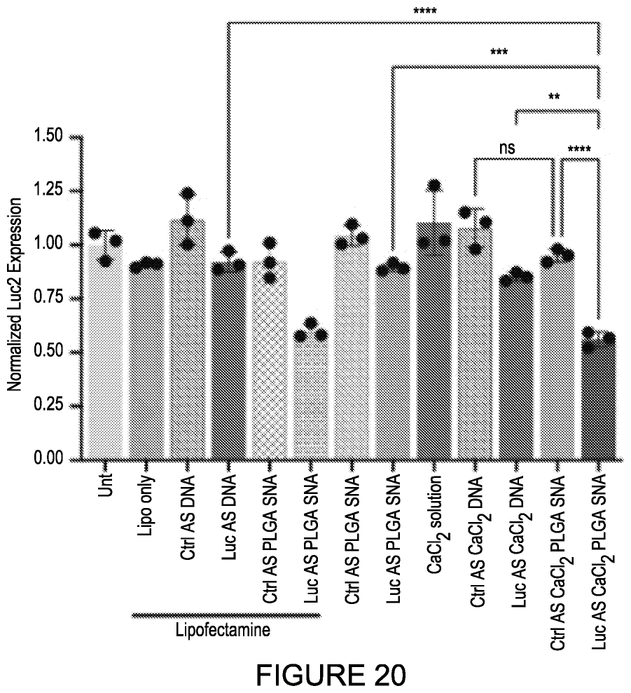

After determining that SNAs salted to 230 mM CaCl₂) could be used to achieve maximal Luc2 silencing, the gene silencing activity of CaCl₂)-salted SNAs was next compared to the analogous linear siRNAs transfected with Lipofectamine™ RNAiMAX or CaCl₂)-siRNAs. When treated at an siRNA concentration of 100 nM for 48 hours, CaCl₂) PLGA SNAs showed a 12-fold greater knockdown compared to PLGA SNAs (94.38±0.86% vs. 6.16±4.02% knockdown, respectively) and a similar level of knockdown to linear siRNAs transfected with Lipofectamine™ RNAiMAX (96.08±0.70% knockdown) (FIG. 13C). CaCl₂) salting effectively enhanced the cytosolic delivery and resulted in increased gene silencing activity of SNAs without inducing any significant cytotoxicity after 48 hours, whereas RNAiMAX is reported to be cytotoxic over this time window.⁸³ CaCl₂) PLGA SNAs also led to greater knockdown than CaCl₂)-siRNAs (76.28±5.39% knockdown), likely due to enhanced cellular uptake and higher affinity for Ca²⁺ ions to the multivalent SNA architecture. While these experiments measured protein knockdown via luminescence measurements, gene silencing was also confirmed at the mRNA level using reverse transcription quantitative polymerase chain reaction (RT-qPCR) (FIG. 19). In order to test if the increased gene knockdown of CaCl₂) PLGA SNAs was limited to siRNAs or if it could be extended to gene regulation via other pathways, CaCl₂) PLGA SNAs functionalized with Luc2 antisense DNA were used to study luciferase gene knockdown in U87-MG-Luc2 cells. Luciferase knockdown efficiency was measured after treating cells with antisense DNA (1 μM) for 48 hours. The results indicated 43.93±3.57% knockdown compared to an untreated control (FIG. 20).

To better understand the enhanced cytosolic delivery of the CaCl₂) PLGA SNAs, U87-MG-Luc2 cells were co-treated with SNAs and bafilomycin A1, which inhibits the ATPase proton (H⁺) pump and prevents endosomal acidification.⁸⁴ Co-treatment with bafilomycin A1 significantly reduced the gene silencing activity of CaCl₂) PLGA SNAs (bafilomycin A1+Luc2 CaCl₂) PLGA SNA: 75.30±1.47% knockdown, Luc2 CaCl₂) PLGA SNA: 97.56±0.57% knockdown, FIG. 13D. Based on this result, it was postulated that Ca²⁺ ions incorporated in the CaCl₂) PLGA SNAs induce the proton sponge effect to enhance the release of SNAs into the cytosol to induce gene silencing.^{39,41-63}

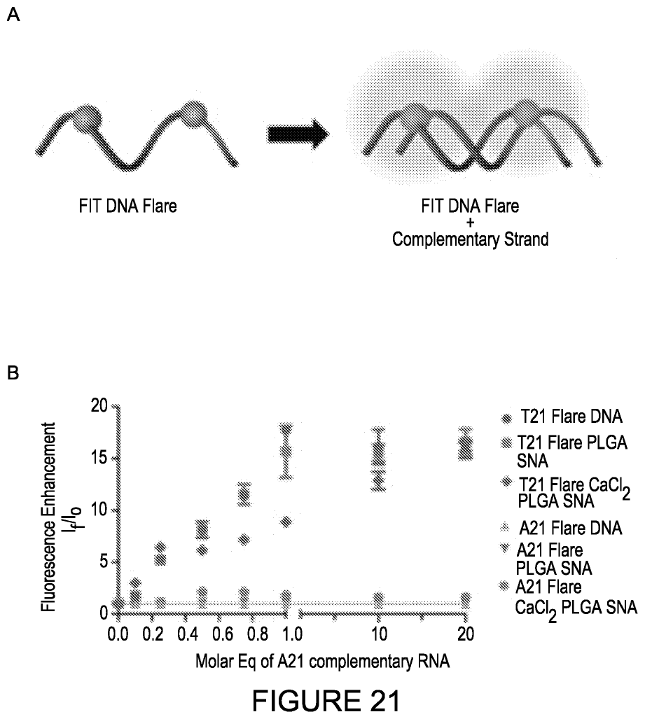

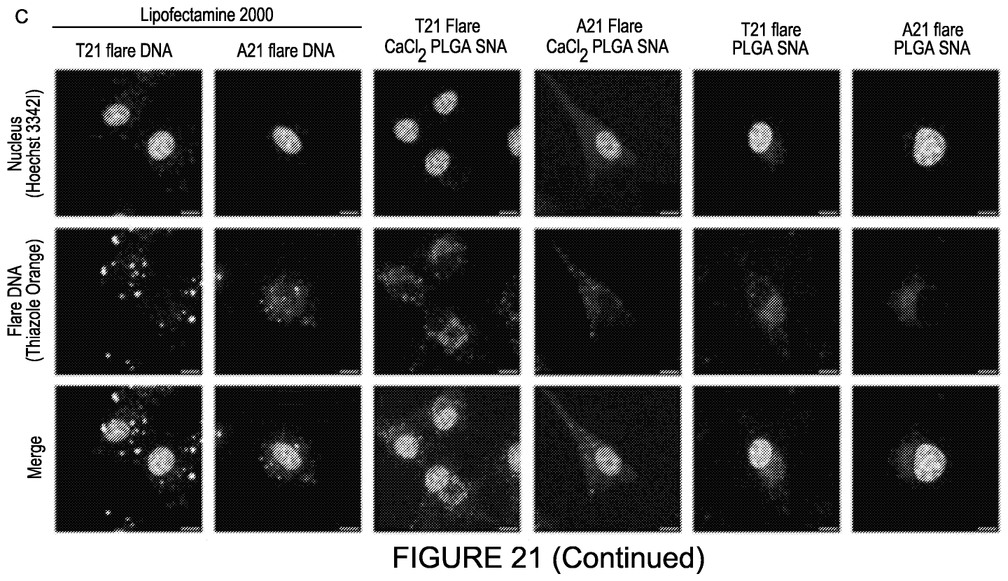

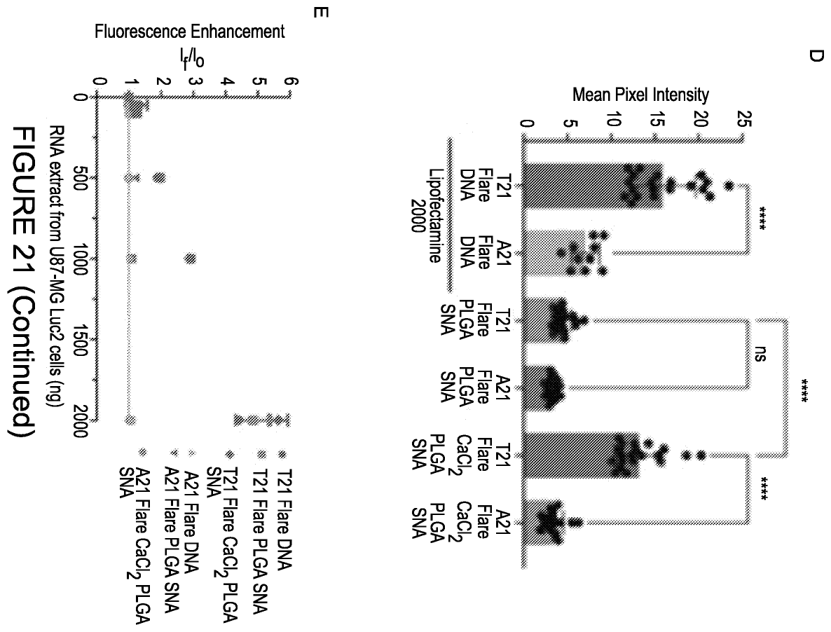

To further determine whether Ca²⁺ complexation to PLGA SNAs can enhance the function of oligonucleotides in applications where cytosolic delivery is essential, PLGA SNAs (Ca²⁺ and Na⁺ forms, as well as linear forms) with thiazole orange (TO)-incorporated poly T21 flare DNA probes, which can bind to the poly A tail of mRNAs within the cytosol⁸⁵, were investigated. Upon T21 flare DNA binding to the poly A tail, TO will undergo forced intercalation (FIT), resulting in fluorescent turn-on due to the restricted rotation of the methine bridge in TO (FIG. 21A).^{67,86-89} First, fluorescent enhancement of linear T21 flare DNAs as well as T21 flare PLGA SNAs and T21 flare CaCl₂) PLGA SNAs was confirmed, where a 15.8 to 16.6-fold fluorescence increase was observed when co-incubated with complementary A21 RNAs (FIG. 21B). Fluorescence enhancement was not observed with control A21 flare DNAs, A21 flare PLGA SNAs, or A21 flare CaCl₂) PLGA SNAs.

Next, U87-MG-Luc2 cells were treated with T21 and A21 linear flare DNAs, flare PLGA SNAs and flare CaCl₂) PLGA SNAs for 2 hours and fluorescent pixel intensity was analyzed by live confocal fluorescence microscopy (FIG. 21C). Cells transfected with linear T21 flare DNAs showed a 2.25±0.53-fold turn-on (increase in mean pixel intensity) compared to cells transfected with control linear A21 flare DNAs (FIG. 21D). Importantly, T21 flare CaCl₂) PLGA SNAs led to a 3.77±0.80-fold fluorescent turn-on in cells compared to the control A21 flare CaCl₂) PLGA SNAs. Note that there was no significant difference in mean pixel intensity observed between cells treated with conventional (NaCl salted) T21 flare PLGA SNAs and A21 flare PLGA SNAs. Collectively, T21 flare CaCl₂) PLGA SNAs exhibited significant fluorescence turn-on compared to conventional T21 flare PLGA, consistent with the conclusion that CaCl₂) PLGA SNAs undergo greater endosomal release into the cytosol compared to conventional PLGA SNAs.

To evaluate the utility of measuring fluorescence enhancement by confocal fluorescence microscopy, an ex cellulo experiment was conducted, incubating T21 and A21 linear DNAs, PLGA SNAs and CaCl₂ PLGA SNAs with varying amounts of total RNA extracted from U87-MG-Luc2 cells. When incubated with total RNA amounts ranging from 500-2000 ng (corresponding to 35,000-150,000 cells worth of total RNA), T21 linear flare DNAs, conventional flare PLGA SNAs and CaCl₂) flare PLGA SNAs all exhibited fluorescence enhancement with linear dependence on the amount of RNA in the range of 2 to 5-fold, which is in agreement with the fluorescence intensity results obtained via confocal microscopy (FIG. 21E).

CaCl₂) PLGA SNAs exhibit enhanced gene regulation activity of therapeutically relevant oncogenes in U87-MG glioblastoma and SK-OV-3 ovarian cancer cell lines. Thus far, it has been determined that CaCl₂) PLGA SNAs downregulate Luc2 protein expression with high potency compared to conventional SNAs. It was next sought to investigate whether CaCl₂) PLGA SNAs could be used to silence other genes to determine if this strategy is broadly applicable to other target sequences. A therapeutically relevant siRNA was used that targets isocitrate dehydrogenase 1 (IDH1), which is upregulated in primary glioblastoma (GBM) cells, inducing increased macromolecular synthesis, promoting aggressive tumor cell progression, and conferring resistance to radiation therapy.^{90,91}

After treating U87-MG cells with IDH1-targeting CaCl₂) PLGA SNAs for 48 hours at 100 nM siRNA concentration, IDH1 protein expression was analyzed by Western blot (FIGS. 14A-B). CaCl₂) PLGA SNAs reduced IDH1 expression by 91.86±3.40%, whereas PLGA SNAs did not induce significant knockdown. CaCl₂) PLGA SNAs also exhibited significantly enhanced knockdown activity compared to CaCl₂)-siRNAs (65.37±4.32% knockdown), likely due to their enhanced uptake. Remarkably, CaCl₂) PLGA SNAs achieved a higher level of knockdown compared to that of the siRNAs delivered with Lipofectamine RNAiMAX (74.78±3.65%), the current standard for siRNA transfection.

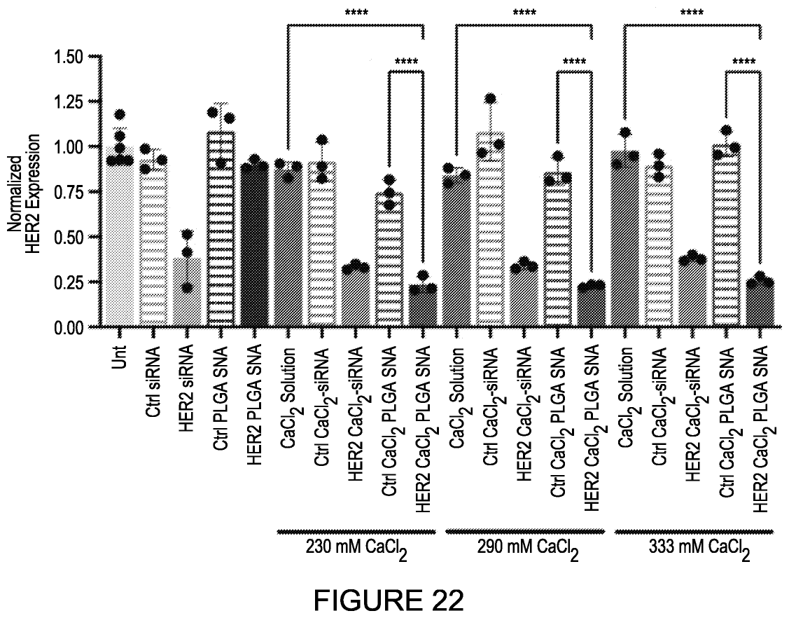

To assess whether CaCl₂) PLGA SNAs can mediate gene regulation in a cell line other than U87-MG, it was sought to knock down human epidermal growth factor receptor 2 (HER2) expression in SK-OV-3 human ovarian cancer cells. HER2 is a well-established oncogene that is involved in accelerated tumor growth, progression, and metastasis.⁹² After treating SK-OV-3 cells with CaCl₂) PLGA SNAs or control groups for 48 hours at a concentration of 100 nM, HER2 protein expression was measured using an in-cell Western blot assay. Consistent with Luc2 and IDH1 protein knockdown results shown in the U87-MG-Luc2 and U87-MG cell lines, respectively, HER2-targeting CaCl₂) PLGA SNAs exhibited 77.22±0.94% reduction in HER2 protein levels while the analogous SNAs resulted in 10% reduction in HER2 protein levels (FIG. 14C). CaCl₂) PLGA SNAs also appeared to induce greater knockdown than CaCl₂)-siRNA complexes (65.99±2.05% knockdown) and Lipofectamine RNAiMAX-transfected siRNA, although the differences were not statistically significant. In these experiments, maximal knockdown was achieved when cells were treated with SNAs salted with a CaCl₂) concentration of 230 mM and the level of knockdown was consistent at higher CaCl₂) concentrations (FIG. 22). Similar to what was seen with the U87-MG cell line, the use of non-targeting CaCl₂) PLGA SNAs or CaCl₂) alone did not result in significant knockdown, indicating that CaCl₂) PLGA SNAs do indeed achieve sequence-specific gene silencing (FIG. 22). In addition, significant cellular toxicity was not observed for cells treated with CaCl₂) PLGA SNAs where SNAs were salted with CaCl₂) concentrations of 230 mM, 290 mM, and 333 mM. (FIG. 14D). These results indicated that CaCl₂) salting enhances the gene silencing activity of SNAs across a variety of different sequences and cell lines without eliciting significant cytotoxicity. Taken together, these results showed that CaCl₂) salting enables SNAs to silence genes more effectively than conventional NaCl-salted SNAs, significantly expanding the applicability of SNAs for a wide range of disease targets for oligonucleotide therapeutics.

### CONCLUSION

This Example showed that by preparing SNAs using CaCl₂) salting, instead of conventional NaCl salting, one can dramatically expand the functionality of SNAs by increasing cytosolic delivery, a capability essential for addressing nucleic acid and small molecule targets that reside in the cytosol or nucleus. Of specific note, CaCl₂) salted PLGA SNAs exhibit a 36-fold increase in cellular uptake at early time points, compared to conventional NaCl salting, and up to an 18-fold enhancement in gene regulation, depending upon antisense or siRNA pathway, with no apparent cytotoxicity. The multivalent nature of the SNA led to enhanced Ca²⁺ binding, allowing the Ca²⁺ to remain associated with the constructs, even under physiological conditions. The data are consistent with the notion that once in the endosomes, the Ca²⁺ ions establish a concentration gradient between the endosome and cytosol that leads to disruption of the endosomal membrane and release of SNAs. Consequently, this approach can be used universally with SNAs, regardless of diagnostic or therapeutic purposes.

### Materials and Methods

All chemicals were purchased from Sigma Aldrich unless otherwise noted.

PLGA/PLGA-PEG-N₃ nanoparticle core (NP) synthesis. PLGA/PLGA-PEG-N₃ nanoparticle cores were synthesized using the nanoprecipitation method with slight modifications.66 PLGA (Resomer® 502H, Sigma Aldrich)/PLGA-PEG-N3 (AI085, Akina Inc) (15.0 mg; 35%, w/w) was co-dissolved in acetonitrile (ACN) (6 mL) then injected dropwise into a 50 mL glass beaker containing 0.3% (v/v) Poloxamer 188 solution (24 mL) and stirred at 900 rpm. The resulting solution was allowed to evaporate for 2 hours in a fume hood. The NP solution was then concentrated to 1 mL using an Amicon filter (15 mL, size cutoff=100K) (EMD Millipore).

Quantifying PLGA/PLGA-PEG-N₃ nanoparticle core concentration. Nanoparticle core concentration was quantified using a NanoSight NS300 (Malvern Instruments). A diluted sample solution (1:10,000 dilution, v/v in nanopure water) of the PLGA-PEG-N₃ core was injected using the NanoSight Sample Assistant (Malvern Instruments, United Kingdom). Each nanoparticle tracking analysis was conducted three times in duplicate using a default script provided by the manufacturer. Nanoparticle concentration was calculated based on the average of triplicate measurements.

Oligonucleotide synthesis. Phosphoramidites and other oligonucleotide reagents were purchased from Glen Research, and RNA oligonucleotides used in this work (Table 2) were synthesized with TOM-RNA reagents on a Mermade 6 system (Bioautomation) according to the manufacture-recommended cleavage and deprotection protocols. All oligonucleotides were purified using reverse-phase high performance liquid chromatography (RP-HPLC) on a Varian Microsorb column (10 μm, 300×10 mm², C4 for Cy5 and DBCO-modified oligonucleotides or C18 for unmodified oligonucleotides) with 0.1 M triethylammonium acetate (TEAA) at pH 7 with a 1% gradient of 100% acetonitrile at a flow rate of 3 mL/min, while monitoring the UV signal of the nucleic acids at 254 nm. After purification, the oligonucleotides were lyophilized, resuspended in UltraPure™ DNase/RNase-free distilled water (Invitrogen), and stored at −80° C. until further use.

TABLE 2
Sequences of RNA oligonucleotides used in this study. SP18: Spacer 18, DBCO:
dibenzocyclooctyne, dT: DNA base thymine dA: DNA base adenine. Bold underlined
text indicates the base location of thiazole orange functionalization.

| Name | Sequence (5′→3′) |
|---|---|
| Luc2 Antisense | GAAGUGCUCGUCCUCGUCCUUdTdT (SEQ ID NO: 8) |
| Luc2 Sense | GGACGAGGACGAGCACUUCUUdTdT (SEQ ID NO: 9) |
| Luc2 Sense DBCO | GGACGAGGACGAGCACUUCUUdTdT-(Sp18)2- |
| modified | DBCO (SEQ ID NO: 10) |
| Luc2 Cy5 Antisense | (Sp18)₂-Cy5-GAAGUGCUCGUCCUCGUCCUUdTdT |
| | (SEQ ID NO: 11) |
| Control (nontargeting) | GAACUUCAGGGUCAGCUUGCCGdTdT (SEQ ID NO: 12) |
| Antisense (GFP) | |
| Control (nontargeting) | GCAAGCUGACCCUGAAGUUCAUdTdT (SEQ ID NO: 13) |
| Sense (GFP) | |
| Control (nontargeting) | GCAAGCUGACCCUGAAGUUCAUdTdT-(Sp18)₂- |
| Sense (GFP) DBCO | DBCO (SEQ ID NO: 14) |
| modified | |
| IDH1 Antisense | UACCCAUCCACUCACAAGCdTdT (SEQ ID NO: 15) |
| IDH1 Sense | GCUUGUGAGUGGAUGGGUAdTdT (SEQ ID NO: 16) |
| IDH1 Sense DBCO | GCUUGUGAGUGGAUGGGUAdTdT-(Sp18)₂-DBCO |
| modified | (SEQ ID NO: 17) |
| HER2 Antisense | UUGGUUGUGAGCGAUGAGCAC (SEQ ID NO: 18) |
| HER2 Sense | GCUCAUCGCUCACAACCAAUU (SEQ ID NO: 19) |
| HER2 Sense DBCO | GCUCAUCGCUCACAACCAAUU-(Sp18)₂-DBCO |
| modified | (SEQ ID NO: 20) |
| T21 Flare DNA DBCO | TTTTTTTTTTTTTTTTTTTTT-(Sp18)₂-DBCO (SEQ ID |
| modified | NO: 21) |

| | |
|---|---|
| A21 Flare DNA DBCO | AAAAAAAAAAAAAAAAAAAAA-(Sp18)₂-DBCO (SEQ |
| modified | ID NO: 22) |
| Luc2 Antisense DNA | TCGAAGATGTTGGGGTGTTTT-(Sp18)₂-DBCO (SEQ |
| DBCO modified | ID NO: 23) |
| Control Antisense DNA | AACACCCCACATCTTCGATTT -(Sp18)₂-DBCO |
| DBCO modified | (SEQ ID NO: 24) |

Synthesis of CaCl₂)-salted PLGA SNAs (CaCl₂) PLGA SNAs). To prepare siRNA duplexes, DBCO-modified sense siRNA strands (20 nmole) and antisense siRNA strands (20 nmole) were hybridized in a duplex buffer (30 mM HEPES and 100 mM potassium acetate, pH 7.5, IDT technologies) by first heating the solution to 95° C. for 2 minutes, then cooling it to 25° C. in a heat block. The concentration of surface azide on the PLGA core was calculated based on previously reported methods.^{66,93} PLGA/PLGA-PEG-N₃ (0.0143 nmol, approximately 500 μL) was added in pH 7.4, 0.1 M HEPES buffered saline, (HBS, 137 mM NaCl) with 0.3% (v/v) Poloxamer 188, and different concentrations of CaCl₂) ranging from 90 mM to 333 mM. Then, 20 nmole of DBCO-modified siRNA duplex was added and the reaction mixture was incubated for 24 hours at room temperature. The un-reacted oligonucleotides were removed by 15 minutes of centrifugation at 10,000×g using a 100 kDa cutoff Amicon spin filter four times using 1×HBS with 0.3% (v/v) Poloxamer 188 with different concentrations of CaCl₂). After the fourth wash, the particles were resuspended in 1×HBS with 0.3% (v/v) Poloxamer 188 with different concentrations of CaCl₂) and was stored until further use for biological studies.

Synthesis of PLGA SNAs (conventional SNAs that are salted with NaCl). PLGA/PLGA-PEG-N₃ (0.0143 nmol, approximately 500 μL) was added in pH 7.4, 0.1M HEPES buffered saline (HBS, 137 mM NaCl) with 0.3% (v/v) Poloxamer 188 and additional 5 M NaCl was added to the solution so that the final concentration of NaCl is adjusted to 500 mM. Then, 20 nmole of DBCO-modified siRNA duplex was added to the reaction mixture, and the sample was incubated for 24 hours at room temperature. The un-reacted oligonucleotides were removed by 15 minutes of centrifugation at 10,000×g using a 100 kDa cutoff Amicon spin filter four times using 1×HBS with 0.3% (v/v) Poloxamer 188. After the fourth wash, the particles were resuspended in 1×HBS with 0.3% (v/v) Poloxamer 188.

Synthesis of Luc2/Control antisense DNA functionalized PLGA SNAs and CaCl₂) salted PLGA SNAs. PLGA/PLGA-PEG-N₃ (0.0143 nmol, approximately 500 μL) was added in pH 7.4, 0.1 M HEPES buffered saline (HBS, 137 mM NaCl) with 0.3% (v/v) Poloxamer 188, with 230 mM of CaCl₂). Then, 20 nmole of DBCO-modified Luc2/Control antisense DNA was added and the reaction mixture was incubated for 24 hours at room temperature. The un-reacted oligonucleotides were removed by 15 minutes of centrifugation at 10,000×g using a 100 kDa cutoff Amicon spin filter four times using 1×HBS with 0.3% (v/v) Poloxamer 188 with 230 mM of CaCl₂). After the fourth wash, the particles were resuspended in 1×HBS with 0.3% (v/v) Poloxamer 188 with 230 mM of CaCl₂). For the synthesis of PLGA SNAs, 500 mM NaCl was used instead of 230 mM CaCl₂).

Quantification of siRNA duplexes functionalized to the PLGA SNAs (NaCl-salted) and CaCl₂)-salted PLGA SNAs (CaCl₂) PLGA SNAs). To quantify the number of antisense siRNA strands functionalized to the SNAs, a previously described method was modified [Barnaby, S. N.; Lee, A.; Mirkin, C. A. Probing the Inherent Stability of SiRNA Immobilized on Nanoparticle Constructs. Proc. Natl. Acad. Sci. U.S.A. 2014, 111, 9739-9744]. Briefly, 20 μL of PLGA SNAs and CaCl₂) PLGA SNAs (in pH 7.4, 0.1M HEPES buffered saline, 0.3% (v/v) Poloxamer 188) were resuspended in 130 μL of 8 M urea and heated to 45° C. for 20 minutes to dehybridize the antisense strands from the DBCO-modified sense strands, which would remain on the SNAs. Then, the solution was centrifuged for 10 minutes at 10,000×g. To confirm that the supernatant contained only the antisense siRNA strands, the supernatant was washed five times with RNase-free water using Amicon spin filters (0.5 mL, size cut-off=3K) for 25 min at 10,000×g to remove the 8 M urea, Poloxamer 188, and 0.1 M HEPES. To measure the concentration of antisense strand, a portion of the supernatant (25 μL) that contains 130 μL 8 M urea and 20 μL HBS, 0.3% (v/v) Poloxamer 188 was analyzed by mixing with Quant-iT OliGreen reagent (Thermo Fisher) with 40 mM EDTA in a 96-well plate and fluorescence was measured using a BioTek Synergy Microplate Reader with excitation/emission wavelengths of 480 nm/520 nm. The concentration was determined based on standard curves of known antisense RNA concentrations incorporating the same concentrations of urea, 1×HBS and Poloxamer 188 as the analyte.

Quantification of Luc2/Control antisense DNA strand functionalized to PLGA SNAs and CaCl₂) PLGA SNAs. To quantify Luc2/Control antisense DNA strands functionalized to the SNAs, SNA was diluted in in pH 7.4, 0.1M HEPES buffered saline, 0.3% (v/v) Poloxamer 188 to 50-fold in a total volume of 100 μL. Then 100 μL of acetonitrile (ACN) was added to dissolve the PLGA core. The portion of solution (25 μL) was analyzed by mixing 100 μL Quant-iT OliGreen reagent (Thermo Fisher) with 40 mM EDTA in a 96-well plate and fluorescence was measured using a BioTek Synergy Microplate Reader with excitation/emission wavelengths of 480 nm/520 nm. The concentration was determined based on standard curves of known antisense DNA concentrations incorporating the same concentrations of ACN, 1×HBS and Poloxamer 188 as the analyte.

Particle size distribution by dynamic light scattering (DLS) and zeta (2) potential measurements. The particle size distribution and the surface charge (zeta potential) of the CaCl₂) PLGA SNAs and PLGA SNAs were measured using a Zetasizer Ultra Red (Malvern Instruments, UK). To measure the size, 1.40 was used as the refractive index.⁶⁶ The hydrodynamic diameter (HD) measurements were derived from the number average value in water at 25° C. The reported DLS size for each sample was based on at least five measurements per run in triplicates. The surface charge (zeta potential) of the particles was measured in triplicates using the DTS 1070 zeta cell (Malvern Instruments, UK), each run was measured in water at 25° C., and 10 to 50 measurements were taken using the automated settings in the ZS Xplorer (Malvern Instruments, UK) software.

PicoGreen exclusion assay to evaluate the association of Ca²⁺ ions within the CaCl₂) PLGA SNAs and Ca²⁺-siRNA complex. The complexation of Ca²⁺ ions in the SNA architecture was determined using a Quant-iT™ PicoGreen™ (Invitrogen) exclusion assay. 50 μL of CaCl₂) PLGA SNAs, PLGA SNAs and Ca²⁺-siRNA complexes (CaCl₂)-siRNAs, that had equal oligonucleotide concentration of 100 nM compared to CaCl₂) PLGA SNAs and PLGA SNAs and equal CaCl₂) concentration of 230 mM compared to CaCl₂) PLGA SNAs) were first added to the 96-well plate and then 150 μL of PicoGreen™ solution containing either 1×HBS or 1×HBS+40 mM EDTA was added to the 96-well plate. The plate was then read at excitation/emission wavelengths of 480 nm/520 nm using a Biotek Cytation 5 plate reader.

PicoGreen exclusion assay to evaluate the dissociation of Ca²⁺ ions within the oligonucleotide shell of the CaCl₂) PLGA SNAs and CaCl₂)-siRNA complex within multiple time points. 300 μL of CaCl₂) PLGA SNAs and CaCl₂)-siRNAs (salted at 230 mM CaCl₂) with same siRNA concentration) was dialyzed against 50 mL of RNase free 0.1 M HEPES, 137 mM NaCl, 1.8 mM CaCl₂) solution at 37° C. using 3.5K MWCO Slide-A-Lyzer™ MINI Dialysis Device (Thermo Fisher) using a 100 ml beaker with a stir bar at 150 rpm. At multiple time points (0 hour, 1 hour, 2 hour, 4 hour, 8 hour and 12 hour), 30 μL of both CaCl₂) PLGA SNA and CaCl₂)-siRNA complex solution was taken out of the dialysis tube. Then 5 μL of the CaCl₂) PLGA SNAs and CaCl₂)-siRNAs was first added to the 96 well plate followed by addition of 95 μL of PicoGreen™ solution containing either 1×HBS or 1×HBS+40 mm EDTA. The plate was then read at excitation/emission wavelengths of 480 nm/520 nm using a Biotek Cytation 5 plate reader. Then the normalized fluorescence (NF) at each time point was calculated by the following equation,

$$\frac{RFU ( Picogreen )}{RFU ( Picogreen + 40 mM EDTA )} .$$

Remaining Ca²⁺ ion associated with the siRNA or SNA at each time point (x hour) then was calculated by

$$100 \% \times ( 1 - \frac{Average {NF}_{t = 0 h} - {NF}_{t = xh}}{Average {NF}_{t = 0 h}} ) .$$

The experiment was performed in triplicates.

Cell culture. The U87-MG glioblastoma cell line (ATCC) was cultured in MEM (Gibco) supplemented with 10% fetal bovine serum (FBS) (Gibco) and 1% penicillin-streptomycin (Thermo Fisher). The U87-MG-Luc2 reporter cell line (ATCC) was cultured in MEM supplemented with 10% FBS, 8 μg/mL blasticidin (Thermo Fisher) and 1% penicillin-streptomycin (Thermo Fisher). The SK-OV-3 ovarian cancer cell line (ATCC) was cultured in DMEM (Gibco) supplemented with 10% FBS and 1% penicillin-streptomycin (Thermo Fisher).

Evaluation of CaCl₂) PLGA SNA and PLGA SNA uptake by flow cytometry. 100,000 (1×10⁵) U87-MG cells were seeded in a 12-well plate in 2 mL of cell culture media and incubated overnight. The cells were then treated with 100 nM by siRNA duplex concentration with the following treatment groups: Cy5-labeled linear siRNA duplex, Cy5-labeled CaCl₂)-siRNA, Cy5-labeled PLGA SNAs, Cy5-labeled CaCl₂) PLGA SNAs (salted to 230 mM CaCl₂)) for 1 hour or 24 hours. As a negative control, cells were pre-treated with 50 μg/mL of fucoidan (Sigma Aldrich), a scavenger receptor A blocker, for 30 minutes. Also, cells were pre-treated with nifedipine, a known calcium ion channel blocker at a concentration of 20 UM for 30 minutes. Then, these cell samples were treated under the above-mentioned conditions (but with the presence of the inhibitors). After 1 hour of incubation, the cells were washed with 1×HBS, trypsinized, and washed twice by centrifugation and resuspension in 1×HBS (300×g for 5 minutes). For live-dead cell staining, LIVE/DEAD™ fixable blue dead cell stain (Invitrogen) solution in 1×HBS buffered saline was used; the cells were incubated with it for 15 minutes at 4° C. The cells were then washed with 1×HBS, fixed using 4% paraformaldehyde solution for 10 minutes, washed and were re-suspended in 1×HBS. The median fluorescence intensity (MFI) of the Cy5 signal was recorded with a FACSymphony™ A3 (BD Biosciences). The experiments were performed in triplicate, and the data were analyzed using FlowJo software (BD Biosciences).

Analysis of intracellular trafficking of PLGA SNAs and CaCl₂) PLGA SNAs by confocal microscopy. To visualize SNA uptake and intracellular trafficking, U87-MG cells were plated on an 8-well chambered coverglass slide (Nunc™ Lab-Tek® II) with a seeding density of 25,000 cells per well with a total volume of 400 μL of cell culture media. After overnight incubation, the cells were treated with CellLight™ Late Endosomes-GFP, BacMam 2.0 (Thermo Fisher) at a particle per cell (PPC) of 40 according to the manufacturer's protocol. After a 24 hour incubation, the cells were treated with Cy5-labeled PLGA SNAs or CaCl₂) PLGA SNAs (salted at 230 mM CaCl₂)) ([siRNA]=100 nM) for 24 hours. The cells were then washed with washing buffer (HBS containing 0.9 mM CaCl₂) and 0.49 mM MgCl₂-6H₂O) three times. Then, the cells were fixed (4% paraformaldehyde) for 15 minutes, washed three times with washing buffer and their nuclei were stained with NucBlue™ Fixed Cell ReadyProbes™ Reagent (DAPI) (Thermo Fisher) according to the manufacturer's protocol. Confocal images of the cells were collected using a Zeiss LSM 800 microscope using equal parameters for image acquisition for each treatment group (e.g., laser power, master gain, offset). Z-stack images (10 slices) of the cells were used to analyze colocalization of Cy5-labeled oligonucleotides within the late endosomes (Rab7a-GFP fusion protein) throughout the entire volume of the cells. Regions of interest (ROI) were assigned by manually tracing the outlines of individual cells. Mander's overlap coefficients (MOCs) were quantified by reconstruction of the Z-stack images of each cell using Zeiss ZEN Blue software.80 Statistical analysis was performed across averages from 10 independent cell images per treatment group.

Cell viability assay. The cell viability of U87-Luc2 cells was determined using PrestoBlue™ cell viability reagent (Thermo Fisher). The cells were seeded in a black, clear-bottom, 96-well plate at a density of 12,000 cells per well. After overnight incubation, the cells were treated CaCl₂) PLGA SNAs with different concentrations of CaCl₂). The final concentration of siRNA treated to cells was kept constant at 100 nM. After treatment for 48 hours, cell viability was measured following the manufacturer's protocol. After incubation, the fluorescence was measured at excitation/emission wavelengths of 560 nm/590 nm using a BioTek Cytation 5 Microplate Reader. The cell viability was normalized to the untreated control and plotted as a percentage of cell viability. The experiment was performed in triplicates, and the error was calculated as the standard deviation of the mean.

Quantification of Luc2 gene knockdown by qPCR. To evaluate Luc2 mRNA expression, U87-MG-Luc2 cells were seeded in a 96-well cell culture plate at a density of 12,000 cells per well with a total volume of 200 μL. To compare the Luc2 mRNA down regulation activity, CaCl₂) PLGA SNAs (salted to 230 mM CaCl₂)), CaCl₂)-siRNAs, or PLGA SNAs were treated to the cells. As positive and negative controls, linear Luc2 and Control siRNA duplex were transfected at a siRNA concentration of 100 nM with Lipofectamine RNAiMAX (Thermo Fisher) following the manufacturer's protocol. After a 48-hour treatment, RNA was isolated from the cells using PureLink RNA Mini Kit (Thermo Fisher). mRNA levels were measured in triplicate using RT-qPCR with qScript XLT One-Step RT-qPCR ToughMix (Quanta Biosciences), TaqMan Gene Expression Assays (Luc2: forward primer: 5′-TAAGGTGGTGGACTTGGACA-3′ (SEQ ID NO: 5); reverse primer: 5′-GTTGTTAACGTAGCCGCTCA-3′ (SEQ ID NO: 6); probe: 5′-FAM-CGCGCTGGTTCACACCCAGT-TAMRA-3′ (SEQ ID NO: 7)), GAPDH: Hs03929097_g1; Thermo Fisher), a Bio-Rad C1000 Touch Thermal Cycler, and a Bio-Rad CFX384 Real-time System. CT values were normalized to the housekeeping gene GAPDH and untreated cells using the Pfaffl method [Pfaffl, M. W. A New Mathematical Model for Relative Quantification in Real-Time RT-PCR. Nucleic Acids Res. 2001, 29, e45].

Quantification of Luc2 protein down regulation by luciferase assay. To assess the functionality of CaCl₂) PLGA SNAs, U87-MG-Luc2 cells were seeded in a in a black, clear-bottom, 96-well plate at a density of 12,000 cells per well with a total volume of 200 μL. After overnight incubation, the cells were treated with CaCl₂) PLGA SNA, non-targeting CaCl₂) PLGA SNAs, and CaCl₂) solution equivalent to concentration that the CaCl₂) PLGA SNA was salted in. The final siRNA concentration of the cells samples for all treatment groups was 100 nM. After a 48-hour treatment, the wells were washed with 1×HBS twice, and 90 μL of fresh cell culture media was added to the wells. Subsequently, 10 μL of PrestoBlue™ Cell viability reagent (Thermo Fisher) was added to measure the relative cell viability compared to the untreated wells. After a 30-minute incubation at 37° C., fluorescence intensity was measured at excitation/emission wavelengths of 560 nm/590 nm using a BioTek Cytation 5 Reader. After measuring the cell viability, the wells were then washed with 150 μL of HBS three times. The luminescence of U87-Luc2 cells were then measured using the Bright-Glo™ Luciferase Assay (Promega) using Biotek Cytation 5 plate reader. Luc2 protein expression was analyzed in arbitrary units where the luminescence value was normalized to the fluorescence value from the PrestoBlue assay. Then, the relative Luc2 expression was normalized to the untreated control group. To compare the Luc2 protein down regulation activity, CaCl₂) PLGA SNAs (salted to 230 mM CaCl₂)) were treated to the cells along with CaCl₂)-siRNAs and PLGA SNAs. As a positive and negative control, linear Luc2 and Control siRNA duplexes were transfected at a siRNA concentration of 100 nM with Lipofectamine RNAiMAX (Thermo Fisher). To minimize cellular cytotoxity for RNAiMAX treated cells, they were washed with PBS and fresh media was added after a 6-hour treatment step. To analyze the effect of endosomal acidification on CaCl₂) PLGA SNA-mediated gene silencing, the cells were pre-treated with bafilomycin A1 at a concentration of 100 nM for 30 minutes. Then, the cells were treated with CaCl₂) PLGA SNAs (salted to 230 mM CaCl₂)) in the presence of the bafilomycin A1.

Quantification of Luc2 protein down regulation by antisense DNA functionalized CaCl₂) PLGA SNAs by luciferase assay. To assess the functionality of antisense DNA functionalized CaCl₂) PLGA SNAs, U87-MG-Luc2 cells were seeded in a in a black, clear-bottom, 96-well plate at a density of 12,000 cells per well with a total volume of 200 μL. After overnight incubation, the cells were treated with CaCl₂) PLGA SNA, non-targeting CaCl₂) PLGA SNAs, and equivalent CaCl₂) solution. The final antisense DNA concentration of the cells samples for all treatment groups was 1 μM. After a 48-hour treatment, the wells were washed with 1×HBS twice, and 90 μL of fresh cell culture media was added to the wells. Subsequently, 10 μL of PrestoBlue™ Cell viability reagent (Thermo Fisher) was added to measure the relative cell viability compared to the untreated wells. After a 30-minute incubation at 37° C., fluorescence intensity was measured at excitation/emission wavelengths of 560 nm/590 nm using a BioTek Synergy Microplate Reader. After measuring the cell viability, the wells were then washed with 150 μL of HBS three times. The luminescence of U87-Luc2 cells were then measured using the Bright-Glo™ Luciferase Assay System (Promega) according to the manufacturer's protocol. Luc2 protein expression was analyzed in arbitrary units where the luminescence value was normalized to the fluorescence value from the PrestoBlue assay. Then, the relative Luc2 expression was normalized to the untreated control group.

Quantification of IDH1 protein down-regulation by Western blot analysis. 100,000 (1×10⁵) U87-MG cells were seeded in a 12-well plate and incubated overnight with a total volume of 2 mL. To compare the IDH1 protein down regulation activity, the cells were treated with CaCl₂) PLGA SNAs (salted to 230 mM CaCl₂)), CaCl₂)-siRNAs or PLGA SNAs. As a positive and negative control treatment group, linear Luc2 and Control siRNA duplex were transfected at a siRNA concentration of 100 nM with Lipofectamine RNAiMAX (Thermo Fisher). To minimize cellular cytotoxity for the RNAiMAX treated cells, they were washed with PBS and fresh media was added after a 6-hour treatment step. After a 48-h incubation, the wells were washed with 1×HBS twice, and the protein lysates were then extracted using radioimmunoprecipitation (RIPA) buffer with halt protease inhibitor cocktail (Thermo Fisher). After measuring protein lysate concentration using a BCA assay reagent (BioRad), 30 μg of protein lysate per treatment sample was separated using 4-12% NuPAGE™ Bis-Tris protein gel (Invitrogen) in 100 V for 70 minutes. Then, the protein gel was transferred to nitrocellulose membrane (Life Technologies) using an iBlot® 2 Gel Transfer Device (Life Technologies). The membranes were blocked with Intercept® (TBS) Blocking Buffer (LI-COR) in room temperature for 1 hour with shaking and incubated overnight at 4° C. with shaking using the following antibodies: rabbit anti-IDH1 (Cell Signaling Technology, 1:1000 dilution in blocking buffer, 10 mL) and mouse IgG1 anti-HSP70 (BD biosciences, 1:2000 dilution in blocking buffer, 10 mL). After the blots were washed with 1×PBST (0.1% Tween-20) three times for 5 minutes, the membranes were incubated with IRDye® 800CW-conjugated goat anti-rabbit secondary antibody (LI-COR, 1:2000 dilution in blocking buffer, 10 mL) and IRDye® 800CW-conjugated goat anti-mouse IgG1 secondary antibody (LI-COR, 1:2000 dilution in blocking buffer, 10 mL) for 1 hour in room temperature with shaking. Then, the nitrocellulose membrane was washed with 1×PBST three times for 5 min. To remove residual Tween-20, the membrane was rinsed in deionized water three times before scanning. Then, the blot image was acquired using an Odyssey® CLx Imager (Li—COR) at 169 μm resolution in the 800-nm fluorescence channel. Then, the band intensity of the blot was quantified using Image J (NIH, Bethesda, MD)⁹⁴ and normalized to the untreated control group. Western blot was performed in triplicate, and the results were plotted in a bar graph, and the error was calculated as the standard deviation of the mean.

Evaluation of HER-2 protein down-regulation by In-cell Western blot analysis. In a black, clear-bottom, 96-well cell culture plate, 10,000 SK-OV-3 cells were seeded with a total volume of 180 μL per well. After overnight incubation, the cells were treated with CaCl₂) PLGA SNAs, the final siRNA concentration in the cells for all treatment groups was 100 nM. PLGA SNAs were salted at a CaCl₂) concentration of 230 mM, 290 mM, and 333 mM. Moreover, CaCl₂)-siRNAs were treated to cells with an equivalent siRNA concentration and CaCl₂) concentrations. 100 nM of linear HER2 siRNA and control siRNA were transfected with lipofectamine RNAiMAX (Thermo Fisher) to the cells as a positive and negative control, respectively. To minimize cellular cytotoxicity for RNAiMAX treated cells, they were washed with PBS and fresh media was added after a 6-hour treatment step. After a 48-hour treatment, the wells were washed with 1×HBS with 4 mM EDTA two times, then 1×PBS, then fixed in methanol chilled at −20° C. for 15 minutes. The wells were then washed with 0.05% Tween-20 in 1×PBS two times, then 1×PBS, then incubated with Intercept® (TBS) Blocking Buffer (LI-COR) for 90 minutes with shaking. The cells were then incubated with HER2 antibody (29D8) (Cell Signaling Technology) diluted 1:200 in Intercept® (TBS) Blocking Buffer for 2 hours. The wells were washed with 0.1% Tween-20 in 1×PBS three times and incubated with 2 μg/mL IRDye® 800CW-conjugated goat anti-rabbit secondary antibody (LI-COR) and 500 nM CellTag 700 (LI-COR) diluted 1:500 in Intercept® blocking buffer for 1 hour protected from light, with shaking. The wells were washed with 0.1% Tween-20 in 1×PBS three times and imaged on an Odyssey CLx system (LI-COR). HER2 protein expression was calculated in arbitrary units by normalizing fluorescence at 800 nm (HER2) to fluorescence at 700 nm (cell viability number). Then, the extent of HER2 protein knockdown was determined by normalizing the HER2 protein expression to that of the untreated control group. Western blot was performed in triplicate, and the results were plotted in a bar graph, and the error was calculated as the standard deviation of the mean.

Synthesis of thiazole orange labeled T21 and A21 flare DNAs. To conjugate thiazole orange to flare DNA sequences, amino-modifier C6 dT and amino modifier C6 dA (Glen Research) was incorporated into the T21 and A21 DNA sequences during the DNA synthesis step, respectively (Table 2). In a typical reaction, 10 μmole (50-fold molar excess) of thiazole orange NHS ester (Glen Research) was first dissolved in 200 μL of DMSO. Then, 200 μL of the DMSO solution was added to the 200 nmole of amino-modified DNA dissolved in 200 μL 0.2 M NaHCO₃ and was incubated in a thermal shaker at 25° C., 700 rpm for 1 hour protected from light. Then, the reaction mixture was run through an NAP™-25 (Cytiva) column according to manufacturer's protocol to remove excess dye. Next, the purified product went through ethanol precipitation, adding 2.5 vol equivalence of ice-cold ethanol and adding 0.3 vol equivalence of 3M sodium acetate then in incubating at −80° C. for 1 hour followed by centrifugation of the precipitation mixture at 21000 g for 30 minutes at 4° C. The supernatant then was discarded, and the pellet formed was washed with 500 μL of ice-cold 70% ethanol twice. After washing, the pellet was lyophilized overnight to remove trace amounts of ethanol and was resuspended in UltraPure™ DNase/RNase-free distilled water (Invitrogen) for further use.

Synthesis of T21/A21 flare DNA functionalized PLGA SNAs and CaCl₂) PLGA SNAs. PLGA/PLGA-PEG-N₃ (0.0143 nmol, approximately 500 μL) was added in pH 7.4, 0.1 M HEPES buffered saline (HBS, 137 mM NaCl) with 0.3% (v/v) Poloxamer 188, with 500 mM of CaCl₂). Then, 2.5 nmole of DBCO-modified T21/A21 flare DNA was added and the reaction mixture was incubated for 24 hours at room temperature. The un-reacted oligonucleotides were removed by 15 minutes of centrifugation at 10,000×g using a 100 kDa cutoff Amicon spin filter four times using 1×HBS with 0.3% (v/v) Poloxamer 188 with 230 mM of CaCl₂). After the fourth wash, the particles were resuspended in 1×HBS with 0.3% (v/v) Poloxamer 188 with 500 mM of CaCl₂). For the synthesis of PLGA SNAs, 500 mM NaCl was used instead of 500 mM CaCl₂).

Quantification of T21/A21 flare strands functionalized to PLGA SNAs and CaCl₂) PLGA SNAs. To quantify T21/A21 flare DNA strands functionalized to the SNAs, SNA was diluted in in pH 7.4, 0.1M HEPES buffered saline, 0.3% (v/v) Poloxamer 188 to 50-fold in a total volume of 100 μL. Then 100 μL of acetonitrile (ACN) was added to dissolve the PLGA core. The portion of solution (25 μL) was analyzed by mixing with 25 μL of 40 mM EDTA in a 96-well plate and fluorescence was measured using a BioTek Cytation 5 plate Reader with excitation/emission wavelengths of 485 nm/528 nm. The concentration was determined based on standard curves of known T21/A21 flare DNA concentrations incorporating the same concentrations of ACN, 1×HBS and Poloxamer 188 as the analyte.

Fluorescence response of linear T21/A21 flare DNA strands, T21/A21 flare PLGA SNAs and T21/A21 flare CaCl₂) PLGA SNAs to complementary A21 RNA strand. 2.5 pmole (by DNA) linear T21/A21 flare DNA strands and T21/A21 flare PLGA SNAs and T21/A21 flare CaCl₂) PLGA SNAs were added to a 96 well plate at a total volume of 50 μL in 1×HBS with 0.3% (v/v) Poloxamer 188 in triplicates. Then varying concentrations of complementary A21 RNA in 1×PBS (0, 0.1, 0.25, 0.50, 0.75, 1.00, 10.00, 20.00 molar excess) were added to the wells that contained flare DNAs and flare PLGA SNAs. Then, the fluorescence reading was taken using the Biotek Cytation 5 plate reader (excitation: 480 nm, emission: 525 nm). The fluorescence turn-on was quantified by the following:

$$\frac{RFU ( Different Molar ratio of A 21 RNA added )}{RFU ( No A 21 RNA added )} .$$

Fluorescence response of linear T21/A21 flare DNA strands and T21/A21 flare PLGA SNAs and T21/A21 flare CaCl₂) PLGA SNAs in U87-MG cells (in cellulo). U87-MG-Luc2 cells were plated on an 8-well chambered coverglass slide (Nunc™ Lab-Tek® II) with a seeding density of 20,000 cells per well with a total volume of 400 μL of cell culture media. After overnight incubation, the cells were treated with T21/A21 flare PLGA SNAs, T21/A21 flare CaCl₂) PLGA SNAs and transfected with T21/A21 linear flare strands with lipofectamine 2000 (Thermo Fisher) at 20 pmole. After a 2 hour incubation, the cells were then washed with washing buffer (HBS containing 0.9 mM CaCl₂) and 0.49 mM MgCl₂-6H₂O) three times. Then, the cell nuclei were labeled with 7.5 μg/mL of Hoechst 33342 trihydrochloride, trihydrate (Invitrogen). Then, the cells were washed with washing buffer three times and then FluroBrite™ DMEM (Gibco) supplemented with 10% FBS and 1% penicillin-streptomycin was added to the wells. Then confocal images of the cells were collected using a Zeiss LSM 800 microscope using equal parameters for image acquisition for each treatment group (e.g., laser power, master gain, offset) and cells were maintained under culturing conditions (37° C., 5% CO₂) under the confocal microscope. Z-stack images (10 slices) of the cells were acquired to analyze the signal of thiazole orange (excitation at 480 nm and emission at 490-530 nm) and Hoechst 33432 (excitation at 350 nm and emission at 460 nm). The acquired Z-stack images were analyzed and processed with Fiji Image J (NIH, Besthada, MD) [Schindelin, J.; Arganda-Carreras, I.; Frise, E.; Kaynig, V.; Longair, M.; Pietzsch, T.; Preibisch, S.; Rueden, C.; Saalfeld, S.; Schmid, B.; Tinevez, J. Y.; White, D. J.; Hartenstein, V.; Eliceiri, K.; Tomancak, P.; Cardona, A. Fiji: An Open-Source Platform for Biological-Image Analysis. Nat. Methods 2012, 9, 676-682] using Z project. Then the mean pixel intensity of each image was calculated, and statistical analysis was performed across averages from 20 independent cell images per treatment group. One-way ANOVA tests with post-hoc Tukey multiple comparison test using Prism Version 9.4.1 (Graphpad).

Fluorescence response of linear T21/A21 flare DNA strands and T21/A21 flare PLGA SNAs and T21/A21 flare CaCl₂) PLGA SNAs to RNA extract from U87-MG cells (ex cellulo). To purify total RNA, U87-MG-Luc2 cells were seeded in an T75 flask with a seeding density of 2,000,000 cells. After overnight incubation, cells were trypsinized and the total number of cells were counted using Countess™ II automated cell counter (Invitrogen). Then the total RNA from the cells was extracted using PureLink™ RNA Mini Kit (Invitrogen) according to manufacturer's protocol in RNase free 1×PBS. The total amount (ng) of RNA extracted then was quantified using NanoDrop 8000 (Thermo Fisher). Based on total number of cells and total RNA extracted, amount of RNA extract that correlates to number of cells (0 ng: 0 cells, 50 ng: 3.75×10³ cells, 100 ng: 7.5×10³ cells, 500 ng: 3.75×10⁴ cells, 1000 ng: 7.5×10⁴ cells and 2000 ng: 1.5×10⁵ cells) of total RNA was added to the 96 well plate in triplicates. Then, 2.5 pmole (by DNA) linear T21/A21 flare DNA strands and T21/A21 flare PLGA SNAs and T21/A21 flare CaCl₂) PLGA SNAs were added to a 96 well plate at a total volume of 50 mL in 1×HBS with 0.3% (v/v) Poloxamer 188 in triplicates. Then, the fluorescence reading was taken using the Biotek Cytation 5 plate reader (excitation: 480 nm, emission: 525 nm). The fluorescence turn-on was quantified by the following,

$$\frac{RFU ( Different amount of RNA extract added )}{RFU ( No RNA extract added )} .$$

Statistical analysis. Significant differences between groups were determined using a student's two tailed t test (Mander's overlap coefficient analysis) and one-way or two-way ANOVA tests with post-hoc Tukey and Sidak multiple comparison test using Prism Version 9.4.1 (Graphpad). Differences were considered statistically significant at p<0.05.

### REFERENCES

(1) Fire, A.; Xu, S.; Montgomery, M. K.; Kostas, S. A.; Driver, S. E.; Mello, C. C. Potent and Specific Genetic Interference by Double-Stranded RNA in Caenorhabditis elegans. Nature 1998, 391, 806-811.
(2) Winkler, J. RNA Interference. Nucleic Acid-Based Drugs 2013, 418, 36-53.
(3) Hammond, S. M.; Bernstein, E.; Beach, D.; Hannon, G. J. An RNA-Directed Nuclease Mediates Post-Transcriptional Gene Silencing in Drosophila Cells. Nature 2000, 404, 293-296.
(4) Aagaard, L.; Rossi, J. J. RNAi Therapeutics: Principles, Prospects and Challenges. Adv. Drug Deliv. Rev. 2007, 59, 75-86.
(5) Gleave, M. E.; Monia, B. P. Antisense Therapy for Cancer. Nat. Rev. Cancer 2005, 5, 468-479.
(6) Mirkin, C. A.; Letsinger, R. L.; Mucic, R. C.; Storhoff, J. J. A DNA-Based Method for Rationally Assembling Nanoparticles into Macroscopic Materials. Nature 1996, 382, 607-609.
(7) Cutler, J. I.; Auyeung, E.; Mirkin, C. A. Spherical Nucleic Acids. J. Am. Chem. Soc. 2012, 134, 1376-1391.
(8) Choi, C. H. J.; Hao, L.; Narayan, S. P.; Auyeung, E.; Mirkin, C. A. Mechanism for the Endocytosis of Spherical Nucleic Acid Nanoparticle Conjugates. Proc. Natl. Acad. Sci. 2013, 110, 7625-7630.
(9) Patel, P. C.; Giljohann, D. A.; Daniel, W. L.; Zheng, D.; Prigodich, A. E.; Mirkin, C. A. Scavenger Receptors Mediate Cellular Uptake of Polyvalent Oligonucleotide-Functionalized Gold Nanoparticles. Bioconjug. Chem. 2010, 21, 2250-2256.
(10) Seferos, D. S.; Prigodich, A. E.; Giljohann, D. A.; Patel, P. C.; Mirkin, C. A. Polyvalent DNA Nanoparticle Conjugates Stabilize Nucleic Acids. Nano Lett. 2009, 9, 308-311.
(11) Rosi, N. L.; Giljohann, D. A.; Thaxton, C. S.; Lytton-Jean, A. K. R.; Han, M. S.; Mirkin, C. A. Oligonucleotide-Modified Gold Nanoparticles for Infracellular Gene Regulation. Science 2006, 312, 1027-1030.
(12) Ding, F.; Mou, Q.; Ma, Y.; Pan, G.; Guo, Y.; Tong, G.; Choi, C. H. J.; Zhu, X.; Zhang, C. A Crosslinked Nucleic Acid Nanogel for Effective SiRNA Delivery and Antitumor Therapy. Angew. Chemie-Int. Ed. 2018, 57, 3064-3068.
(13) Patel, P. C.; Giljohann, D. A.; Seferos, D. S.; Mirkin, C. A. Peptide Antisense Nanoparticles. Proc. Natl. Acad. Sci. U.S.A. 2008, 105, 17222-17226.
(14) Giljohann, D. A.; Seferos, D. S.; Prigodich, A. E.; Patel, P. C.; Mirkin, C. A. Gene Regulation with Polyvalent SiRNA-Nanoparticle Conjugates. J. Am. Chem. Soc. 2009, 131, 2072-2073.
(15) Ruan, W.; Zheng, M.; An, Y.; Liu, Y.; Lovejoy, D. B.; Hao, M.; Zou, Y.; Lee, A.; Yang, S.; Lu, Y.; Morsch, M.; Chung, R.; Shi, B. DNA Nanoclew Templated Spherical Nucleic Acids for SIRNA Delivery. Chem. Commun. 2018, 54, 3609-3612.
(16) Fakih, H. H.; Katolik, A.; Malek-Adamian, E.; Fakhoury, J. J.; Kaviani, S.; Damha, M. J.; Sleiman, H. F. Design and Enhanced Gene Silencing Activity of Spherical 2′-Fluoroarabinose Nucleic Acids (FANA-SNAs). Chem. Sci. 2021, 12, 2993-3003.
(17) Fakih, H. H.; Fakhoury, J. J.; Bousmail, D.; Sleiman, H. F. Minimalist Design of a Stimuli-Responsive Spherical Nucleic Acid for Conditional Delivery of Oligonucleotide Therapeutics. ACS Appl. Mater. Interfaces 2019, 11, 13912-13920.
(18) Young, K. L.; Scott, A. W.; Hao, L.; Mirkin, S. E.; Liu, G.; Mirkin, C. A. Hollow Spherical Nucleic Acids for Intracellular Gene Regulation Based upon Biocompatible Silica Shells. Nano Lett. 2012, 12, 3867-3871.
(19) Zheng, D.; Giljohann, D. A.; Chen, D. L.; Massich, M. D.; Wang, X. Q.; Iordanov, H.; Mirkin, C. A.; Paller, A. S. Topical Delivery of SiRNA-Based Spherical Nucleic Acid Nanoparticle Conjugates for Gene Regulation. Proc. Natl. Acad. Sci. U.S.A. 2012, 109, 11975-11980.
(20) Nemati, H.; Ghahramani, M. H.; Faridi-Majidi, R.; Izadi, B.; Bahrami, G.; Madani, S. H.; Tavoosidana, G. Using SiRNA-Based Spherical Nucleic Acid Nanoparticle Conjugates for Gene Regulation in Psoriasis. J. Control. Release 2017, 268, 259-268.
(21) Jensen, S. A.; Day, E. S.; Ko, C. H.; Hurley, L. A.; Luciano, J. P.; Kouri, F. M.; Merkel, T. J.; Luthi, A. J.; Patel, P. C.; Cutler, J. I.; Daniel, W. L.; Scott, A. W.; Rotz, M. W.; Meade, T. J.; Giljohann, D. A.; Mirkin, C. A.; Stegh, A. H. Spherical Nucleic Acid Nanoparticle Conjugates as an RNAi-Based Therapy for Glioblastoma. Sci. Transl. Med. 2013, 5, 209ra152-209ra152.
(22) Kumthekar, P.; Ko, C. H.; Paunesku, T.; Dixit, K.; Sonabend, A. M.; Bloch, O.; Tate, M.; Schwartz, M.; Zuckerman, L.; Lezon, R.; Lukas, R. V.; Jovanovic, B.; McCortney, K.; Colman, H.; Chen, S.; Lai, B.; Antipova, O.; Deng, J.; Li, L.; Tommasini-Ghelfi, S.; Hurley, L. A.; Unruh, D.; Sharma, N. V.; Kandpal, M.; Kouri, F. M.; Davuluri, R. V.; Brat, D. J.; Muzzio, M.; Glass, M.; Vijayakumar, V.; Heidel, J.; Giles, F. J.; Adams, A. K.; James, C. D.; Woloschak, G. E.; Horbinski, C.; Stegh, A. H. A First-in-Human Phase 0 Clinical Study of RNA Interference-Based Spherical Nucleic Acids in Patients with Recurrent Glioblastoma. Sci. Transl. Med. 2021, 13.
(23) Mou, Q.; Ma, Y.; Ding, F.; Gao, X.; Yan, D.; Zhu, X.; Zhang, C. Two-in-One Chemogene Assembled from Drug-Integrated Antisense Oligonucleotides To Reverse Chemoresistance. J. Am. Chem. Soc. 2019, 141, 6955-6966.
(24) Chen, L.; Li, G.; Wang, X.; Li, J.; Zhang, Y. Spherical Nucleic Acids for Near-Infrared Light-Responsive Self-Delivery of Small-Interfering RNA and Antisense Oligonucleotide. ACS Nano 2021, 15, 11929-11939.
(25) Randeria, P. S.; Seeger, M. A.; Wang, X.-Q.; Wilson, H.; Shipp, D.; Mirkin, C. A.; Paller, A. S. SiRNA-Based Spherical Nucleic Acids Reverse Impaired Wound Healing in Diabetic Mice by Ganglioside GM3 Synthase Knockdown. Proc. Natl. Acad. Sci. 2015, 112, 5573-5578.
(26) Calabrese, C. M.; Merkel, T. J.; Briley, W. E.; Randeria, P. S.; Narayan, S. P.; Rouge, J. L.; Walker, D. A.; Scott, A. W.; Mirkin, C. A. Biocompatible Infinite-Coordination-Polymer Nanoparticle-Nucleic-Acid Conjugates for Antisense Gene Regulation. Angew. Chemie-Int. Ed. 2015, 54, 476-480.
(27) Banga, R. J.; Chernyak, N.; Narayan, S. P.; Nguyen, S. T.; Mirkin, C. A. Liposomal Spherical Nucleic Acids. J. Am. Chem. Soc. 2014, 136, 9866-9869.
(28) Vasher, M. K.; Yamankurt, G.; Mirkin, C. A. Hairpin-like SiRNA-Based Spherical Nucleic Acids. J. Am. Chem. Soc. 2022, 144, 3174-3181.
(29) Sita, T. L.; Kouri, F. M.; Hurley, L. A.; Merkel, T. J.; Chalastanis, A.; May, J. L.; Ghelfi, S. T.; Cole, L. E.; Cayton, T. C.; Barnaby, S. N.; Sprangers, A. J.; Savalia, N.; James, C. D.; Lee, A.; Mirkin, C. A.; Stegh, A. H. Dual Bioluminescence and Near-Infrared Fluorescence Monitoring to Evaluate Spherical Nucleic Acid Nanoconjugate Activity in Vivo. Proc. Natl. Acad. Sci. 2017, 114, 201702736.
(30) Wang, X.; Hao, L.; Bu, H. F.; Scott, A. W.; Tian, K.; Liu, F.; De Plaen, I. G.; Liu, Y.; Mirkin, C. A.; Tan, X. Di. Spherical Nucleic Acid Targeting MicroRNA-99b Enhances Intestinal MFG-E8 Gene Expression and Restores Enterocyte Migration in Lipopolysaccharide-Induced Septic Mice. Sci. Rep. 2016, 6, 1-13.
(31) Liu, H.; Kang, R. S.; Bagnowski, K.; Yu, J. M.; Radecki, S.; Daniel, W. L.; Anderson, B. R.; Nallagatla, S.; Schook, A.; Agarwal, R.; Giljohann, D. A.; Paller, A. S. Targeting the IL-17 Receptor Using Liposomal Spherical Nucleic Acids as Topical Therapy for Psoriasis. J. Invest. Dermatol. 2020, 140, 435-444.e4.
(32) Ponedal, A.; Zhu, S.; Sprangers, A. J.; Wang, X. Q.; Yeo, D. C.; Lio, D. C. S.; Zheng, M.; Capek, M.; Narayan, S. P.; Meckes, B.; Paller, A. S.; Xu, C.; Mirkin, C. A. Attenuation of Abnormal Scarring Using Spherical Nucleic Acids Targeting Transforming Growth Factor Beta 1. ACS Appl. Bio Mater. 2020, 3, 8603-8610.
(33) Jiang, T.; Qiao, Y.; Ruan, W.; Zhang, D.; Yang, Q.; Wang, G.; Chen, Q.; Zhu, F.; Yin, J.; Zou, Y.; Qian, R.; Zheng, M.; Shi, B. Cation-Free SiRNA Micelles as Effective Drug Delivery Platform and Potent RNAi Nanomedicines for Glioblastoma Therapy. Adv. Mater. 2021, 33, 1-10.
(34) Jiang, K.; Zhao, D.; Ye, R.; Liu, X.; Gao, C.; Guo, Y.; Zhang, C.; Zeng, J.; Wang, S.; Song, J. Transdermal Delivery of Poly-Hyaluronic Acid-Based Spherical Nucleic Acids for Chemogene Therapy. Nanoscale 2022, 14, 1834-1846.
(35) Chou, L.; Callmann, C. E.; Dominguez, D.; Zhang, B.; Mirkin, C. A. Disrupting the Interplay between Programmed Cell Death Protein 1 and Programmed Death Ligand 1 with Spherical Nucleic Acids in Treating Cancer. ACS Cent. Sci. 2022, 8, 1299-1305.
(36) Daniel, W. Clinical Development of AST-005, a Topically Applied Antisense Spherical Nucleic Acid for the Treatment of Psoriasis. In Proceedings of TIDES: Oligonucleotide and Peptide Therapeutics; Munich, Germany, 2017.
(37) Giljohann, D. Clinical Results for XCUR17, a Topically Applied Anitsense Spherical Nucleic Acid in Patients with Psoriasis. In Proceedings of TIDES: Oligonucleotide and Peptide Therapeutics; San Diego, CA, USA, 2019.
(38) Shi, J.; Kantoff, P. W.; Wooster, R.; Farokhzad, O. C. Cancer Nanomedicine: Progress, Challenges and Opportunities. Nat. Rev. Cancer 2017, 17, 20-37.
(39) Selby, L. I.; Cortez-Jugo, C. M.; Such, G. K.; Johnston, A. P. R. Nanoescapology: Progress toward Understanding the Endosomal Escape of Polymeric Nanoparticles. Wiley Interdiscip. Rev. Nanomedicine Nanobiotechnology 2017, 9.
(40) Wu, X. A.; Choi, C. H. J.; Zhang, C.; Hao, L.; Mirkin, C. A. Intracellular Fate of Spherical Nucleic Acid Nanoparticle Conjugates. J. Am. Chem. Soc. 2014, 136, 7726-7733.
(41) Boussif, O.; LezoualC'H, F.; Zanta, M. A.; Mergny, M. D.; Scherman, D.; Demeneix, B.; Behr, J. P. A Versatile Vector for Gene and Oligonucleotide Transfer into Cells in Culture and in Vivo: Polyethylenimine. Proc. Natl. Acad. Sci. U.S.A. 1995, 92, 7297-7301.
(42) Sonawane, N. D.; Szoka, F. C.; Verkman, A. S. Chloride Accumulation and Swelling in Endosomes Enhances DNA Transfer by Polyamine-DNA Polyplexes. J. Biol. Chem. 2003, 278, 44826-44831.
(43) Yang, S.; May, S. Release of Cationic Polymer-DNA Complexes from the Endosome: A Theoretical Investigation of the Proton Sponge Hypothesis. J. Chem. Phys. 2008, 129.
(44) Patil, M. L.; Zhang, M.; Taratula, O.; Garbuzenko, O. B.; He, H.; Minko, T. Internally Cationic Polyamidoamine PAMAM-OH Dendrimers for SIRNA Delivery: Effect of the Degree of Quaternization and Cancer Targeting. Biomacromolecules 2009, 10, 258-266.
(45) Godbey, W. T.; Wu, K. K.; Mikos, A. G. Poly(Ethylenimine) and Its Role in Gene Delivery. J. Control. Release 1999, 60, 149-160.
(46) Godbey, W. T.; Wu, K. K.; Mikos, A. G. Poly(Ethylenimine)-Mediated Gene Delivery Affects Endothelial Cell Function and Viability. Biomaterials 2001, 22, 471-480.
(47) Graham, F. L.; van der Eb, A. J. A New Technique for the Assay of Infectivity of Human Adenovirus 5 DNA. Virology 1973, 52, 456-467.
(48) Kakizawa, Y.; Furukawa, S.; Kataoka, K. Block Copolymer-Coated Calcium Phosphate Nanoparticles Sensing Intracellular Environment for Oligodeoxynucleotide and SIRNA Delivery. J. Control. Release 2004, 97, 345-356.
(49) Pittella, F.; Cabral, H.; Maeda, Y.; Mi, P.; Watanabe, S.; Takemoto, H.; Kim, H. J.; Nishiyama, N.; Miyata, K.; Kataoka, K. Systemic SiRNA Delivery to a Spontaneous Pancreatic Tumor Model in Transgenic Mice by PEGylated Calcium Phosphate Hybrid Micelles. J. Control. Release 2014, 178, 18-24.
(50) Neuhaus, B.; Frede, A.; Westendorf, A. M.; Epple, M. Gene Silencing of the Pro-Inflammatory Cytokine TNF-α with SiRNA Delivered by Calcium Phosphate Nanoparticles, Quantified by Different Methods. J. Mater. Chem. B 2015, 3, 7186-7193.
(51) Gerasimenko, J. V.; Tepikin, A. V.; Petersen, O. H.; Gerasimenko, O. V. Calcium Uptake via Endocytosis with Rapid Release from Acidifying Endosomes. Curr. Biol. 1998, 8, 1335-1338.
(52) Li, J.; Chen, Y. C.; Tseng, Y. C.; Mozumdar, S.; Huang, L. Biodegradable Calcium Phosphate Nanoparticle with Lipid Coating for Systemic SiRNA Delivery. J. Control. Release 2010, 142, 416-421.
(53) Chen, C.; Okayama, H. High-Efficiency Transformation of Mammalian Cells by Plasmid DNA. Mol. Cell. Biol. 1987, 7, 2745-2752.
(54) Craig, A. M. Transfecting Cultured Neurons. Cult. nerve cells 1998, 2, 79-111.
(55) Jiang, M.; Chen, G. High Ca²⁺-Phosphate Transfection Efficiency in Low-Density Neuronal Cultures. Nat. Protoc. 2006, 1, 695-700.
(56) Kingston, R. E.; Chen, C. A.; Okayama, H. Calcium Phosphate Transfection. Curr. Protoc. Neurosci. 1997, 1, A.1C.1-A.1C.8.
(57) Washbourne, P.; McAllister, A. K. Techniques for Gene Transfer into Neurons. Curr. Opin. Neurobiol. 2002, 12, 566-573.
(58) Sokolova, V. V.; Radtke, I.; Heumann, R.; Epple, M. Effective Transfection of Cells with Multi-Shell Calcium Phosphate-DNA Nanoparticles. Biomaterials 2006, 27, 3147-3153.
(59) Seelos, C. A Critical Parameter Determining the Aging of DNA-Calcium-Phosphate Precipitates. Anal. Biochem. 1997, 245, 109-111.
(60) Lo, C. W.; Lin, T.; Ueno, M.; Romero-Lopez, M.; Maruyama, M.; Kohno, Y.; Rhee, C.; Yao, Z.; Pérez-Cruz, M.; Meyer, E.; Goodman, S. B. Optimization and Characterization of Calcium Phosphate Transfection in Mesenchymal Stem Cells. Tissue Eng.—Part C Methods 2019, 25, 543-552.
(61) Sun, B.; Gillard, M.; Wu, Y.; Wu, P.; Xu, Z. P.; Gu, W. Bisphosphonate Stabilized Calcium Phosphate Nanoparticles for Effective Delivery of Plasmid DNA to Macrophages. ACS Appl. Bio Mater. 2020, 3, 986-996.
(62) Goldshtein, M.; Forti, E.; Ruvinov, E.; Cohen, S. Mechanisms of Cellular Uptake and Endosomal Escape of Calcium-SiRNA Nanocomplexes. Int. J. Pharm. 2016, 515, 46-56.
(63) Ruvinov, E.; Kryukov, O.; Forti, E.; Korin, E.; Goldstein, M.; Cohen, S. Calcium-SiRNA Nanocomplexes: What Reversibility Is All About. J. Control. Release 2015, 203, 150-160.
(64) Gilding, D. K.; Reed, A. M. Biodegradable Polymers for Use in Surgery-Polyglycolic/Poly(Actic Acid) Homo- and Copolymers: 1. Polymer (Guildf). 1979, 20, 1459-1464.
(65) Danhier, F.; Ansorena, E.; Silva, J. M.; Coco, R.; Le Breton, A.; Préat, V. PLGA-Based Nanoparticles: An Overview of Biomedical Applications. J. Control. Release 2012, 161, 505-522.
(66) Zhu, S.; Xing, H.; Gordiichuk, P.; Park, J.; Mirkin, C. A. PLGA Spherical Nucleic Acids. Adv. Mater. 2018, 30, 1-6.
(67) Ebrahimi, S. B.; Samanta, D.; Cheng, H. F.; Nathan, L. I.; Mirkin, C. A. Forced Intercalation (FIT)-Aptamers. J. Am. Chem. Soc. 2019, 141, 13744-13748.
(68) Ambia-Garrido, J.; Vainrub, A.; Pettitt, B. M. A Model for Structure and Thermodynamics of SsDNA and DsDNA near a Surface: A Coarse Grained Approach. Comput. Phys. Commun. 2010, 181, 2001-2007.
(69) Govender, T.; Stolnik, S.; Garnett, M. C.; Illum, L.; Davis, S. S. PLGA Nanoparticles Prepared by Nanoprecipitation: Drug Loading and Release Studies of a Water Soluble Drug. J. Control. Release 1999, 57, 171-185.
(70) Yan, F.; Zhang, C.; Zheng, Y.; Mei, L.; Tang, L.; Song, C.; Sun, H.; Huang, L. The Effect of Poloxamer 188 on Nanoparticle Morphology, Size, Cancer Cell Uptake, and Cytotoxicity. Nanomedicine Nanotechnology, Biol. Med. 2010, 6, 170-178.
(71) Geall, A. J.; Blagbrough, I. S. Rapid and Sensitive Ethidium Bromide Fluorescence Quenching Assay of Polyamine Conjugate-DNA Interactions for the Analysis of Lipoplex Formation in Gene Therapy. J. Pharm. Biomed. Anal. 2000, 22, 849-859.
(72) Dragan, A. I.; Bishop, E. S.; Casas-Finet, J. R.; Strouse, R. J.; Schenerman, M. A.; Geddes, C. D. Metal-Enhanced PicoGreen Fluorescence: Application for Double-Stranded DNA Quantification. Anal. Biochem. 2010, 396, 8-12.
(73) Ahn, S. J.; Costa, J.; Emanuel, J. R. PicoGreen Quantitation of DNA: Effective Evaluation of Samples Pre- or Post-PCR. Nucleic Acids Res. 1996, 24, 2623-2625.
(74) Dragan, A. I.; Casas-Finet, J. R.; Bishop, E. S.; Strouse, R. J.; Schenerman, M. A.; Geddes, C. D. Characterization of PicoGreen Interaction with DsDNA and the Origin of Its Fluorescence Enhancement upon Binding. Biophys. J. 2010, 99, 3010-3019.
(75) Lariccia, V.; Fine, M.; Magi, S.; Lin, M. J.; Yaradanakul, A.; Llaguno, M. C.; Hilgemann, D. W. Massive Calcium-Activated Endocytosis without Involvement of Classical Endocytic Proteins. J. Gen. Physiol. 2011, 137, 111-132.
(76) Bronner, F. Extracellular and Intracellular Regulation of Calcium Homeostasis. ScientificWorldJournal. 2001, 1, 919-925.
(77) Weiss, J. H.; Hartley, D. M.; Koh, J.; Choi, D. W. The Calcium Channel Blocker Nifedipine Attenuates Slow Excitatory Amino Acid Neurotoxicity. Science (80-.). 1990, 247, 1474-1477.
(78) Zamponi, G. W.; Striessnig, J.; Koschak, A.; Dolphin, A. C. The Physiology, Pathology, and Pharmacology of Voltage-Gated Calcium Channels and Their Future Therapeutic Potential. Pharmacol. Rev. 2015, 67, 821-870.
(79) Cataldi, M.; Perez-Reyes, E.; Tsien, R. W. Differences in Apparent Pore Sizes of Low and High Voltage-Activated Ca²⁺ Channels. J. Biol. Chem. 2002, 277, 45969-45976.
(80) Manders, E. M. M.; Stap, J.; Brakenhoff, G. J.; Van Driel, R.; Aten, J. A. Dynamics of Three-Dimensional Replication Patterns during the S-Phase, Analysed by Double Labelling of DNA and Confocal Microscopy. J. Cell Sci. 1992, 103, 857-862.
(81) Ghibelli, L.; Cerella, C.; Diederich, M. The Dual Role of Calcium as Messenger and Stressor in Cell Damage, Death, and Survival. Int. J. Cell Biol. 2010, 2010.
(82) Tseng, Y. C.; Yang, A.; Huang, L. How Does the Cell Overcome LCP Nanoparticle-Induced Calcium Toxicity? Mol. Pharm. 2013, 10, 4391-4395.
(83) Berardo, C.; Siciliano, V.; Di Pasqua, L. G.; Richelmi, P.; Vairetti, M.; Ferrigno, A. Comparison between Lipofectamine RNAiMAX and Genmute Transfection Agents in Two Cellular Models of Human Hepatoma. Eur. J. Histochem. 2019, 63, 189-194.
(84) Johnson, L. S.; Dunn, K. W.; Pytowski, B.; McGraw, T. E. Endosome Acidification and Receptor Trafficking: Bafilomycin A1 Slows Receptor Externalization by a Mechanism Involving the Receptor's Internalization Motif. Mol. Biol. Cell 1993, 4, 1251-1266.
(85) Kubota, T.; Ikeda, S.; Yanagisawa, H.; Yuki, M.; Okamoto, A. Hybridization-Sensitive Fluorescent Probe for Long-Term Monitoring of Intracellular RNA. Bioconjug. Chem. 2009, 20, 1256-1261.
(86) Köhler, O.; Jarikote, D. V.; Seitz, O. Forced Intercalation Probes (FIT Probes): Thiazole Orange as a Fluorescent Base in Peptide Nucleic Acids for Homogeneous Single-Nucleotide-Polymorphism Detection. ChemBioChem 2005, 6, 69-77.
(87) Hövelmann, F.; Gaspar, I.; Chamiolo, J.; Kasper, M.; Steffen, J.; Ephrussi, A.; Seitz, O. LNA-Enhanced DNA FIT-Probes for Multicolour RNA Imaging. Chem. Sci. 2016, 7, 128-135.
(88) Hövelmann, F.; Bethge, L.; Seitz, O. Single Labeled DNA FIT Probes for Avoiding False-Positive Signaling in the Detection of DNA/RNA in QPCR or Cell Media. ChemBioChem 2012, 13, 2072-2081.
(89) Samanta, D.; Ebrahimi, S. B.; Kusmierz, C. D.; Cheng, H. F.; Mirkin, C. A. Protein Spherical Nucleic Acids for Live-Cell Chemical Analysis. J. Am. Chem. Soc. 2020, 142, 13350-13355.
(90) Calvert, A. E.; Chalastanis, A.; Wu, Y.; Hurley, L. A.; Kouri, F. M.; Bi, Y.; Kachman, M.; May, J. L.; Bartom, E.; Hua, Y.; Mishra, R. K.; Schiltz, G. E.; Dubrovskyi, O.; Mazar, A. P.; Peter, M. E.; Zheng, H.; James, C. D.; Burant, C. F.; Chandel, N. S.; Davuluri, R. V.; Horbinski, C.; Stegh, A. H. Cancer-Associated IDH1 Promotes Growth and Resistance to Targeted Therapies in the Absence of Mutation. Cell Rep. 2017, 19, 1858-1873.
(91) Wahl, D. R.; Dresser, J.; Wilder-Romans, K.; Parsels, J. D.; Zhao, S. G.; Davis, M.; Zhao, L.; Kachman, M.; Wernisch, S.; Burant, C. F.; Morgan, M. A.; Feng, F. Y.; Speers, C.; Lyssiotis, C. A.; Lawrence, T. S. Glioblastoma Therapy Can Be Augmented by Targeting IDH1-Mediated NADPH Biosynthesis. Cancer Res. 2017, 77, 960-970.
(92) Lanitis, E.; Dangaj, D.; Hagemann, I. S.; Song, D. G.; Best, A.; Sandaltzopoulos, R.; Coukos, G.; Powell, D. J. Primary Human Ovarian Epithelial Cancer Cells Broadly Express HER2 at Immunologically-Detectable Levels. PLOS One 2012, 7.
(93) Luk, B. T.; Jack Hu, C. M.; Fang, R. H.; Dehaini, D.; Carpenter, C.; Gao, W.; Zhang, L. Interfacial Interactions between Natural RBC Membranes and Synthetic Polymeric Nanoparticles. Nanoscale 2014, 6, 2730-2737.
(94) Schindelin, J.; Arganda-Carreras, I.; Frise, E.; Kaynig, V.; Longair, M.; Pietzsch, T.; Preibisch, S.; Rueden, C.; Saalfeld, S.; Schmid, B.; Tinevez, J. Y.; White, D. J.; Hartenstein, V.; Eliceiri, K.; Tomancak, P.; Cardona, A. Fiji: An Open-Source Platform for Biological-Image Analysis. Nat. Methods 2012, 9, 676-682.

1. A spherical nucleic acid (SNA) comprising:
(a) a nanoparticle core; and
(b) a shell of oligonucleotides attached to the external surface of the nanoparticle core, wherein one or more oligonucleotides in the shell of oligonucleotides comprises a phosphate backbone;
the SNA comprising Ca²⁺ ions adsorbed to one or more oligonucleotides in the shell of oligonucleotides.

2. (canceled)

3. The SNA of claim 1, wherein Ca²⁺ ions are adsorbed to the phosphate backbone of one or more oligonucleotides in the shell of oligonucleotides.

4. (canceled)

5. The SNA of claim 1, wherein Ca²⁺ ions are adsorbed to one or more nucleobases of one or more oligonucleotides in the shell of oligonucleotides.

6. The SNA of claim 1, wherein about, at least about, or less than about 5%, about, at least about, or less than about 10%, about, at least about, or less than about 15%, about, at least about, or less than about 20%, about, at least about, or less than about 25%, about, at least about, or less than about 30%, about, at least about, or less than about 35%, about, at least about, or less than about 40%, about, at least about, or less than about 45%, about, at least about, or less than about 50%, about, at least about, or less than about 55%, about, at least about, or less than about 60%, about, at least about, or less than about 65%, about, at least about, or less than about 70%, about, at least about, or less than about 75%, about, at least about, or less than about 80%, about, at least about, or less than about 85%, about, at least about, or less than about 90%, about, at least about, or less than about 95%, or about or less than about 100% of the total available Ca²⁺ binding sites on a SNA are occupied by a Ca²⁺ ion.

7. The SNA of claim 1, wherein the SNA has a zeta potential that is about −40 millivolts (mV) to about −10 mV.

8. (canceled)

9. The SNA of claim 1, wherein the nanoparticle core is a metallic core, a semiconductor core, an insulator core, an upconverting core, a micellar core, a dendrimer core, a liposomal core, a lipid nanoparticle core, a polymer core, a metal-organic framework core, a protein core, or a combination thereof.

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. The SNA of claim 1, wherein the shell of oligonucleotides comprises an inhibitory oligonucleotide, an immunostimulatory oligonucleotide, a targeting oligonucleotide, or a combination thereof.

38. (canceled)

39. (canceled)

40. (canceled)

41. (canceled)

42. (canceled)

43. (canceled)

44. (canceled)

45. (canceled)

46. (canceled)

47. (canceled)

48. A composition comprising a plurality of the spherical nucleic acids (SNAs) of claim 1.

49. (canceled)

50. A method of making a calcium chloride (CaCl₂)) salted spherical nucleic acid (SNA), the SNA comprising:
(a) a nanoparticle core; and
(b) a shell of oligonucleotides attached to the external surface of the nanoparticle core, wherein each oligonucleotide in the shell of oligonucleotides comprises a phosphate backbone;
the method comprising:
combining the nanoparticle core, a plurality of oligonucleotides, and calcium chloride (CaCl₂)) to create a mixture, wherein the combining results in the plurality of oligonucleotides becoming attached to the nanoparticle core to create the shell of oligonucleotides attached to the external surface of the nanoparticle core, thereby resulting in the CaCl₂) salted SNA,
and then optionally isolating the CaCl₂) salted SNA from the mixture.

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. The method of claim 50, wherein the shell of oligonucleotides comprises an inhibitory oligonucleotide, an immunostimulatory oligonucleotide, or a combination thereof.

72. (canceled)

73. (canceled)

74. (canceled)

75. (canceled)

76. (canceled)

77. (canceled)

78. (canceled)

79. (canceled)

80. (canceled)

81. (canceled)

82. A composition comprising a plurality of the CaCl₂) salted spherical nucleic acids (SNAs) produced by the method of claim 50.

83. (canceled)

84. A method of inhibiting expression of a gene product comprising the step of hybridizing a target polynucleotide encoding the gene product with the spherical nucleic acid (SNA) of claim 1, wherein hybridizing between the target polynucleotide and one or more oligonucleotides in the shell of oligonucleotides occurs over a length of the target polynucleotide with a degree of complementarity sufficient to inhibit expression of the gene product.

85. (canceled)

86. (canceled)

87. (canceled)

88. (canceled)

89. (canceled)

90. A method of treating a disorder comprising administering an effective amount of the SNA of claim 1 to a subject in need thereof, wherein the administering treats the disorder.

91. The method of claim 90, wherein the disorder is cancer, an infectious disease, an autoimmune disease, or a combination thereof.

92. A method for detecting a target analyte comprising the step of contacting the target analyte with the SNA of claim 1, wherein one or more oligonucleotides in the shell of oligonucleotides comprises a detectable marker, wherein the contacting results in binding of the target analyte to the one or more oligonucleotides in the shell of oligonucleotides comprising the detectable marker, resulting in a detectable change and thereby detecting the target analyte.

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. A method for up-regulating activity of a toll-like receptor (TLR), comprising contacting a cell having the toll-like receptor with the SNA of claim 1, thereby up-regulating activity of the TLR.

106. The method of claim 105 wherein the shell of oligonucleotides comprises one or more oligonucleotides that is a TLR agonist.

107. (canceled)

108. A method for down-regulating activity of a toll-like receptor (TLR), comprising contacting a cell having the toll-like receptor with the SNA of claim 1, thereby down-regulating activity of the TLR.

109. (canceled)

110. (canceled)

111. (canceled)

112. (canceled)

113. A method of inhibiting expression of a gene product comprising the step of contacting a cell comprising the gene product with the spherical nucleic acid (SNA) of claim 1, thereby inhibiting expression of the gene product.

114. (canceled)

115. (canceled)

116. (canceled)

117. (canceled)

118. The method of claim 113, wherein accumulation of the spherical nucleic acid (SNA) of claim 1 within an endosome is reduced by at least 5%, at least 10%, at least 15%, at least 20%, at least 25%, or more compared to accumulation of NaCl-salted SNAs within the endosome.


SEQUENCE LISTING (21 sequences)

SEQ ID NO: 1 - DNA, 20 bp, synthetic construct
  source 1..20: mol_type = other DNA
tccatgacgttcctgacgtt

SEQ ID NO: 2 - DNA, 20 bp, synthetic construct
  misc_feature 20: note = Spacer-18 (hexaethyleneglycol))2Cholesterol
  source 1..20: mol_type = other DNA
tccatgacgttcctgacgtt

SEQ ID NO: 3 - DNA, 24 bp, synthetic construct
  source 1..24: mol_type = other DNA
tcgtcgttttgtcgttttgtcgtt

SEQ ID NO: 4 - DNA, 24 bp, synthetic construct
  misc_feature 24: note = (Spacer-18 (hexaethyleneglycol))2Cholesterol
  source 1..24: mol_type = other DNA
tcgtcgttttgtcgttttgtcgtt

SEQ ID NO: 5 - DNA, 20 bp, synthetic construct
  source 1..20: mol_type = other DNA
taaggtggtggacttggaca

SEQ ID NO: 6 - DNA, 20 bp, synthetic construct
  source 1..20: mol_type = other DNA
gttgttaacgtagccgctca

SEQ ID NO: 7 - DNA, 20 bp, synthetic construct
  source 1..20: mol_type = other DNA
cgcgctggttcacacccagt

SEQ ID NO: 8 - RNA, 23 nt, synthetic construct
  source 1..23: mol_type = other RNA
  modified_base 5: mod_base = OTHER; note = uracil
  modified_base 8: mod_base = OTHER; note = uracil
  modified_base 11: mod_base = OTHER; note = uracil
  modified_base 14: mod_base = OTHER; note = uracil
  modified_base 17: mod_base = OTHER; note = uracil
  modified_base 20..21: mod_base = OTHER; note = uracil
gaagtgctcgtcctcgtcctttt

SEQ ID NO: 9 - RNA, 23 nt, synthetic construct
  source 1..23: mol_type = other RNA
  modified_base 17..18: mod_base = OTHER; note = uracil
  modified_base 20..21: mod_base = OTHER; note = uracil
ggacgaggacgagcacttctttt

SEQ ID NO: 10 - RNA, 23 nt, synthetic construct
  source 1..23: mol_type = other RNA
  modified_base 17..18: mod_base = OTHER; note = uracil
  modified_base 20..21: mod_base = OTHER; note = uracil
  misc_feature 23: note = (Sp-18)2
ggacgaggacgagcacttctttt

SEQ ID NO: 11 - RNA, 23 nt, synthetic construct
  source 1..23: mol_type = other RNA
  modified_base 5: mod_base = OTHER; note = uracil
  modified_base 8: mod_base = OTHER; note = uracil
  modified_base 11: mod_base = OTHER; note = uracil
  modified_base 14: mod_base = OTHER; note = uracil
  modified_base 17: mod_base = OTHER; note = uracil
  modified_base 20..21: mod_base = OTHER; note = uracil
gaagtgctcgtcctcgtcctttt

SEQ ID NO: 12 - RNA, 24 nt, synthetic construct
  source 1..24: mol_type = other RNA
  modified_base 5..6: mod_base = OTHER; note = uracil
  modified_base 12: mod_base = OTHER; note = uracil
  modified_base 17..18: mod_base = OTHER; note = uracil
gaacttcagggtcagcttgccgtt

SEQ ID NO: 13 - RNA, 24 nt, synthetic construct
  source 1..24: mol_type = other RNA
  modified_base 7: mod_base = OTHER; note = uracil
  modified_base 13: mod_base = OTHER; note = uracil
  modified_base 18..19: mod_base = OTHER; note = uracil
  modified_base 21: mod_base = OTHER; note = uracil
gcaagctgaccctgaagttcattt

SEQ ID NO: 14 - RNA, 24 nt, synthetic construct
  source 1..24: mol_type = other RNA
  modified_base 7: mod_base = OTHER; note = uracil
  modified_base 13: mod_base = OTHER; note = uracil
  modified_base 18..19: mod_base = OTHER; note = uracil
  modified_base 22: mod_base = OTHER; note = uracil
  misc_feature 24: note = (Sp18)2
gcaagctgaccctgaagttcattt

SEQ ID NO: 15 - RNA, 20 nt, synthetic construct
  source 1..20: mol_type = other RNA
  modified_base 1: mod_base = OTHER; note = uracil
  modified_base 7: mod_base = OTHER; note = uracil
  modified_base 12: mod_base = OTHER; note = uracil
tcccatccactcacaagctt

SEQ ID NO: 16 - RNA, 20 nt, synthetic construct
  source 1..20: mol_type = other RNA
  modified_base 3..4: mod_base = OTHER; note = uracil
  modified_base 6: mod_base = OTHER; note = uracil
  modified_base 10: mod_base = OTHER; note = uracil
  modified_base 14: mod_base = OTHER; note = uracil
  modified_base 18: mod_base = OTHER; note = uracil
gcttggagtggatgggtatt

SEQ ID NO: 17 - RNA, 21 nt, synthetic construct
  source 1..21: mol_type = other RNA
  modified_base 3..4: mod_base = OTHER; note = uracil
  modified_base 6: mod_base = OTHER; note = uracil
  modified_base 10: mod_base = OTHER; note = uracil
  modified_base 14: mod_base = OTHER; note = uracil
  modified_base 18: mod_base = OTHER; note = uracil
  misc_feature 21: note = (Sp-18)2
gcttgtgagtggatgggtatt

SEQ ID NO: 18 - RNA, 21 nt, synthetic construct
  source 1..21: mol_type = other RNA
  modified_base 1..2: mod_base = OTHER; note = uracil
  modified_base 5..6: mod_base = OTHER; note = uracil
  modified_base 8: mod_base = OTHER; note = uracil
  modified_base 15: mod_base = OTHER; note = uracil
ttggttgtgagcgatgagcac

SEQ ID NO: 19 - RNA, 21 nt, synthetic construct
  source 1..21: mol_type = other RNA
  modified_base 3: mod_base = OTHER; note = uracil
  modified_base 6: mod_base = OTHER; note = uracil
  modified_base 10: mod_base = OTHER; note = uracil
  modified_base 20..21: mod_base = OTHER; note = uracil
gctcatcgctcacaaccaatt

SEQ ID NO: 20 - RNA, 21 nt, synthetic construct
  source 1..21: mol_type = other RNA
  modified_base 3: mod_base = OTHER; note = uracil
  modified_base 6: mod_base = OTHER; note = uracil
  modified_base 10: mod_base = OTHER; note = uracil
  modified_base 20..21: mod_base = OTHER; note = uracil
  misc_feature 21: note = (Sp18)2
gctcatcgctcacaaccaatt

SEQ ID NO: 21 - DNA, 21 bp, synthetic construct
  source 1..21: mol_type = other DNA
  misc_feature 21: note = (Sp18)2
ttttttttttttttttttttt